(19) United States
(12) Patent Application Publication
(10) Pub. No.: US 2025/0129117 A1
(43) Pub. Date: Apr. 24, 2025

(54) RAPID PURIFICATION OF MONOCLONAL ANTIBODY FROM IN-PROCESS UPSTREAM CELL CULTURE MATERIAL

(71) Applicant: Regeneron Pharmaceuticals, Inc., Tarrytown, NY (US)

(72) Inventors: Paul Conroy, Laois (IE); Denislav Andonov Chavdarov, Limerick (IE)

(21) Appl. No.: 18/918,283

(22) Filed: Oct. 17, 2024

Related U.S. Application Data
(60) Provisional application No. 63/708,147, filed on Oct. 16, 2024.
(60) Provisional application No. 63/544,767, filed on Oct. 18, 2023.

(51) Int. Cl. C07K 1/22; C07K 16/22; C07K 16/28; G01N 33/68

(52) U.S. Cl. CPC C07K 1/22; C07K 16/22; C07K 16/2866; G01N 33/6854; C07K 2317/14

(57) ABSTRACT

The present invention generally pertains to methods of enriching an antibody of interest. In particular, the present invention pertains to the use of Protein A chromatography in the form of a centrifugal column to enrich a therapeutic antibody from an upstream cell culture process for product quality attribute profiling.

### CROSS-REFERENCE TO RELATED APPLICATIONS

This application claims the benefit of U.S. Provisional Patent Application No. 63/544,767, filed on Oct. 18, 2023 and U.S. Provisional Patent Application No. 63/708,147, filed on Oct. 16, 2024 each of which is incorporated herein by reference in its entirety.

### BACKGROUND

Biophysical properties of therapeutic peptides and proteins can affect their safety, efficacy and shelf-life. For example, the presence of different size variants or charge variants may alter protein solubility, binding, and stability. Variations in attributes that are important for product quality may be referred to as critical product quality attributes.

Therapeutic peptides or proteins, such as antibodies, may acquire different variants and become heterogeneous due to various post-translation modifications (PTMs), protein degradation, enzymatic modifications, and chemical modifications. These alterations to biophysical properties may occur at almost any point during and after peptide and protein production. Because these alterations to biophysical characteristics may affect the safety, efficacy, and shelf-life of therapeutic peptides and proteins, it is important to identify different variants for particular therapeutic peptides or proteins as early as possible.

In order to characterize product quality attributes of a therapeutic peptide or protein, a sufficient amount of peptide or protein must first be enriched in a sample. For therapeutic antibodies, enrichment or purification is generally performed using Protein A chromatography. However, conventional Protein A chromatography methods require large volumes of material and may be both time- and resource-intensive. Conventional large-scale methods are not compatible with rapid or high-throughput analysis of material throughout manufacturing and development, particularly during the upstream cell culture process.

Therefore, it will be appreciated that a need exists for systems and methods to enrich, purify, and/or characterize therapeutic peptides or proteins in a rapid and high-throughput fashion at any stage of cell culture, manufacturing or development.

### SUMMARY

This disclosure provides methods for enriching a protein of interest from a cell culture sample.

In some exemplary embodiments, the methods can comprise (a) contacting a cell culture sample including a protein of interest to a centrifugal column including an affinity resin to produce an immobilized sample, wherein the affinity resin specifically binds to the protein of interest; (b) subjecting the immobilized sample to at least one washing step; and (c) subjecting the immobilized sample from (b) to at least one elution step to produce an enriched protein of interest.

In one aspect, the protein of interest is selected from a group consisting of a therapeutic protein, a receptor, an antigen-binding protein, an antibody, a monoclonal antibody, a multispecific antibody, a bispecific antibody, an antibody-derived protein, a fusion protein, a receptor fusion protein, a trap protein, a fragment thereof, a variant thereof, and a combination thereof.

In one aspect, the protein of interest is a monoclonal antibody.

In one aspect, the protein of interest is dupilumab.

In one aspect, the protein of interest is aflibercept.

In one aspect, the cell culture sample is a mammalian cell culture or an insect cell culture. In another aspect, the cell culture sample is from a CHO cell culture, a CHO-K1 cell culture, a BHK cell culture, a HEK 293 cell culture, a Sf9 insect cell culture, or a variation thereof.

In one aspect, the cell culture sample is a clarified cell culture sample.

In one aspect, the cell culture sample is taken from a cell culture at a day from day 1 to day 20, day 3 to day 15, day 3 to day 12, day 3 to day 10, day 5 to day 10, day 5 to day 12, or day 5 to day 13.

In one aspect, the cell culture sample is taken from a cell culture at a day selected from a group consisting of day 1, 2, 3, 4, 5, 6, 7, 8, 9, 10, 11, 12, 13, 14, 15, 16, 17, 18, 19, or 20.

In one aspect, the method further comprises repeating steps (a)-(c) at least once. In a specific aspect, the method is repeated using at least a first cell culture sample and a second cell culture sample taken from the same cell culture. In a more specific aspect, the first cell culture sample is taken at a first day and the second cell culture sample is taken at a second day. In another specific aspect, the first cell culture sample and the second cell culture sample are taken with a time between samples of about 3 hours, about 6 hours, about 12 hours, about 18 hours, about 24 hours, about 1 day, about 2 days, about 3 days, about 4 days, about 5 days, about 6 days, about 7 days, about 8 days, about 9 days, or about 10 days.

In one aspect, the method further comprises performing steps (a)-(c) with at least two cell culture samples in parallel. In a specific aspect, the at least two cell culture samples are from two different cell cultures.

In one aspect, the contacting step comprises combining the cell culture sample and a binding buffer. In a specific aspect, the binding buffer comprises Tris-buffered saline, sodium phosphate, HEPES, or Tris. In a more specific aspect, the binding buffer further comprises sodium chloride or calcium chloride. In a specific aspect, the binding buffer comprises sodium phosphate. In a more specific aspect, the binding buffer further comprises sodium chloride. In another specific aspect, a pH of the binding buffer is from about 6 to about 8.

In one aspect, the contacting step comprises adding to the column a combined volume of a binding buffer and the cell culture sample of from 250 to 1000 μL, from 300 to 900 μL, from 400 to 800 μL, from 500 to 700 μL from 550 to 650 μL, from 590 to 610 μL, from 599 to 601 μL, or about 600 μL.

In one aspect, the contacting step comprises adding to the column a volume of the cell culture sample of from 50 to 100 μL, from 60 to 90 μL, from 70 to 80 μL, about 70 μL, about 71 μL, about 72 μL, about 73 μL, about 74 μL, about 75 μL, about 76 μL, about 77 μL, about 78 μL, about 79 μL, or about 80 μL.

In one aspect, the contacting step comprises adding to the column an amount of protein of from 100.5 μg to 804 μg, 250 μg to 1 g, from 350 μg to 900 μg, from 450 μg to 804 μg, from 500 μg to 700 μg, from 550 μg to 650 μg, from 575 μg to 625 μg, from 590 μg to 610 μg, about 595 μg, about 596 μg, about 597 μg, about 598 μg, about 599 μg, about 600 μg, about 601 μg, about 602 μg, about 603 μg, about 604 μg, or about 605 μg.

In one aspect, the affinity resin is Protein A resin, Protein G resin, or a combination thereof.

In one aspect, the at least one washing step comprises adding a washing buffer to the column and centrifuging the column to produce a washed flowthrough. In a specific aspect, the washing buffer comprises Tris-buffered saline, sodium phosphate, sodium acetate, HEPES, or Tris. In a more specific aspect, the washing buffer further comprises sodium chloride or calcium chloride. In a specific aspect, the washing buffer comprises sodium phosphate or sodium acetate. In a more specific aspect, the washing buffer further comprises sodium chloride. In another specific aspect, a pH of the washing buffer is from about 6 to about 8. In another specific aspect, a volume of the washing buffer is about 600 μL. In yet another specific aspect, the centrifuging is performed at about 100 relative centrifugal force (RCF). In still another specific aspect, the centrifuging is performed for about 1 minute.

In one aspect, a number of washing steps is one, two, or three. In another aspect, a number of washing step is two. In yet another aspect, the washing buffer of the first washing step comprises sodium phosphate and sodium chloride, and the washing buffer of the second washing step comprises sodium acetate.

In one aspect, the at least one elution step comprises adding an elution buffer to the column and centrifuging the column to produce an eluate. In a specific aspect, the elution buffer comprises acetic acid or glycine. In a more specific aspect, a concentration of the acetic acid is about 0.24% or about 40 mM. In a more specific aspect, a concentration of the acetic acid is about 0.125% or about 20 mM. In another specific aspect, a concentration of the glycine is about 0.1 M. In yet another specific aspect, a volume of the elution buffer is about 400 μL. In an additional specific aspect, a pH of the elution buffer is from 1 to 4, from 2 to 4, from 2.5 to 3.5, from 2.8 to 3.2, about 1, about 1.5, about 2, about 2.5, about 3, or about 3.5. In still another specific aspect, the centrifuging is performed at about 100 RCF. In a further specific aspect, the centrifuging is performed for about 1 minute.

In one aspect, a number of elution steps is one, two, or three.

In one aspect, the at least one elution step comprises adding a neutralizing buffer to the column. In a specific aspect, the neutralizing buffer comprises Tris base. In a more specific aspect, a concentration of the Tris base is from 1 M to 2 M, about 1 M, about 1.5 M, or about 2 M. In another specific aspect, a volume of the neutralizing buffer is from 5 μL to 50 μL, about 5 μL, about 10 μL, about 20 μL, about 30 μL, about 40 μL, or about 50 μL

In one aspect, a yield of the enriched protein of interest is above 50%, above 60%, above 70%, above 80%, above 90%, above 95%, above 99%, about 60%, about 70%, about 75%, about 80%, about 85%, about 90%, about 91%, about 92%, about 93%, about 94%, about 95%, about 96%, about 97%, about 98%, about 99%, or about 100%.

In one aspect, an amount of protein in the enriched protein of interest is above 10 μg, above 20 μg, above 50 μg, above 100 μg, above 200 μg, above 300 μg, above 400 μg, above 500 μg, above 600 μg, above 700 μg, above 800 μg, above 900 μg, above 1000 μg, about 10 μg, about 20 μg, about 50 μg, about 100 μg, about 200 μg, about 300 μg, about 400 μg, about 500 μg, about 600 μg, about 700 μg, about 800 μg, about 900 μg, or about 1000 μg.

In one aspect, a concentration of the enriched protein of interest is above 0.01 μg/μL, above 0.05 μg/μL, above 0.1 μg/μL, above 0.2 μg/μL, above 0.5 μg/μL, above 1 μg/μL, above 2 μg/μL, about 0.05 μg/μL, about 0.1 μg/μL, about 0.2 μg/μL, about 0.5 μg/μL, about 1 μg/μL, about 1.5 μg/μL, about 2 μg/μL, or about 2.5 μg/μL.

In one aspect, a duration of the method is less than 24 hours, less than 12 hours, less than 6 hours, less than 3 hours, less than 2 hours, less than 1 hour, less than 30 minutes, about 3 hours, about 2 hours, about 1.5 hours, about 1 hour, about 50 minutes, about 45 minutes, about 40 minutes, about 30 minutes, or about 20 minutes.

In another aspect, the method further comprises characterizing at least one product quality attribute of the enriched protein of interest. In yet another aspect, the method further comprises subjecting the enriched protein of interest to chromatography, mass spectrometry, spectroscopy, capillary electrophoresis, gel electrophoresis, and/or a ligand binding assay.

In one aspect, the method further comprises characterizing at least one size variant of the enriched protein of interest. In another aspect, the method further comprises characterizing at least one high molecular weight species of the enriched protein of interest. In a specific aspect, the characterizing comprises subjecting the enriched protein of interest to size exclusion chromatography (SEC) analysis.

In one aspect, the method further comprises characterizing at least one fragment of the enriched protein of interest. In a specific aspect, the characterizing comprises subjecting the enriched protein of interest to capillary electrophoresis with sodium dodecyl sulfate (CE-SDS) analysis.

In one aspect, the method further comprises characterizing at least one charge variant of the enriched protein of interest. In a specific aspect, the characterizing comprises subjecting the enriched protein of interest to imaged capillary isoelectric focusing electrophoresis (iCIEF).

In one aspect, the method further comprises characterizing at least one glycan of the enriched protein of interest. In a specific aspect, the characterizing comprises subjecting the enriched protein of interest to hydrophilic interaction chromatography (HILIC) analysis.

In a specific aspect, the method further comprises using the at least one product quality attribute to determine whether the cell culture should be continued or discontinued. In another specific aspect, the method further comprises using the at least one product quality attribute to determine whether the cell culture should be modified.

These, and other, aspects of the present invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 illustrates a general production process for a recombinant protein such as a monoclonal antibody (mAb), according to an exemplary aspect.

FIG. 2 illustrates steps of a Protein A chromatography method, according to an exemplary aspect.

FIG. 3 illustrates aggregation pathways for monoclonal antibodies, according to an exemplary aspect.

FIG. 4 illustrates immunoglobin fragmentation, according to an exemplary aspect.

FIG. 5 shows upstream stages of an antibody manufacturing process from which samples were sourced for the methods and systems of the present invention, according to an exemplary aspect.

FIG. 6A illustrates a workflow of the Protein A chromatography methods and systems of the present invention, according to an exemplary aspect.

FIG. 6B illustrates a workflow of one step of the Protein A chromatography methods and systems of the present invention, according to an exemplary aspect.

FIG. 7A shows protein gained based on incoming protein for each Protein A column load, according to an exemplary aspect.

FIG. 7B shows protein yield based on incoming protein for each Protein A column load, according to an exemplary aspect.

FIG. 8 illustrates N-glycan structures of an enriched antibody analyzed using hydrophilic interaction chromatography (HILIC), according to an exemplary aspect.

FIG. 9A shows protein yield of dupilumab based on theoretical column load as measured by UV-visible spectroscopy, according to an exemplary aspect.

FIG. 9B shows protein yield of dupilumab based on theoretical column load as measured by titer assay, according to an exemplary aspect.

FIG. 9C shows a comparison of protein yield of dupilumab based on theoretical column load as measured by UV-visible spectroscopy or titer assay, according to an exemplary aspect.

FIG. 10 shows a product quality profile of dupilumab purified by the methods and systems of the present invention compared to a conventional Protein A method, according to an exemplary aspect.

FIG. 11 shows results from aggregation testing of dupilumab purified by the methods and systems of the present invention from different small bioreactors compared to a conventional small-scale purification method, according to an exemplary aspect.

FIG. 12 shows the charge variant profile of dupilumab purified by the methods and systems of the present invention from different small bioreactors compared to a conventional small-scale purification method, according to an exemplary aspect.

FIG. 13 shows results from fragmentation testing of dupilumab purified by the methods and systems of the present invention from different small bioreactors compared to a conventional small-scale purification method, according to an exemplary aspect.

FIG. 14 shows a product quality profile of aflibercept purified by the methods and systems of the present invention using in-house buffer compared to a conventional small-scale purification method using the manufacturer's buffers, according to an exemplary aspect.

FIG. 15A shows the average overall step yield percentage versus total column load (μg), according to an exemplary aspect.

FIG. 15B shows the average step yield percentage for eluate 1 versus total column load (μg), according to an exemplary aspect.

FIG. 16 shows results from aggregation testing of aflibercept purified by the methods and systems of the present invention at various total column loading levels compared to a conventional small-scale purification method, according to an exemplary aspect.

FIG. 17 shows the charge variant profile of aflibercept purified by the methods and systems of the present invention at various total column loading levels compared to a conventional small-scale purification method, according to an exemplary aspect.

FIG. 18 shows results from aggregation testing of aflibercept purified by the methods and systems of the present invention with cell culture samples collected at various time points compared to a conventional small-scale purification method, according to an exemplary aspect.

FIG. 19 shows the charge variant profile of aflibercept purified by the methods and systems of the present invention with cell culture samples collected at various time points compared to a conventional small-scale purification method, according to an exemplary aspect.

### DETAILED DESCRIPTION

Analysis of critical product quality attributes of biotherapeutics is essential for ensuring the safety, identity, strength, purity, and quality of drug products delivered to patients. For the production of a therapeutic antibody, exemplary product quality attributes include size variation (including aggregation and fragmentation), charge variation, and glycosylation variation. Assessment of these product quality attributes requires a sufficiently enriched sample, which is typically prepared using Protein A chromatography. However, conventional methods use large-scale Protein A chromatography that requires the preparation of a large volume of cell culture material, can only handle one sample at a time, and is time- and resource-intensive. A large-scale Protein A method may, for example, require a week of processing time and on the order of 10,000 L of cell culture material, while a lab-scale Protein A method may, for example, require several days of processing time and on the order of 250 L of cell culture material.

It would be advantageous to characterize protein quality attributes of a biotherapeutic earlier in the production process, during upstream cell culture. It would further be advantageous to be able to enrich a therapeutic antibody from a small amount of material, to do so rapidly (on the scale of minutes or hours instead of days), and to do so with multiple samples in parallel. In order to meet these needs, novel systems and methods were developed for rapid small-scale enrichment and purification of biotherapeutics from upstream cell culture samples, which are described in detail below.

One advantage of the disclosed invention is the ability to enrich a biotherapeutic, for example, dupilumab, at a small scale, for example on the order of tens or hundreds of μL of cell culture fluid from any day of cell culture, and on the order of hundreds of μg of protein. Another advantage is the ability to enrich a biotherapeutic on a short time scale, for example in around 30 minutes or on the order of hours, as opposed to on the order of days in a lab-scale or large-scale system. Many samples may be enriched simultaneously, since a single centrifuge can process dozens of centrifuge tubes at a time, thereby allowing replicates or comparators to be processed in parallel.

The rapid, small-scale capabilities of the disclosed invention allow for enrichment of a biotherapeutic throughout the upstream cell culture process, and thereby a time course of product quality attributes throughout the process. Early and/or regular monitoring of product quality attributes may inform cell culture parameters, and may inform whether a product batch should be continued or discontinued, which may save a large amount of time and resources compared to waiting for results from a large-scale enrichment and downstream quality analysis. Regular monitoring may also be useful for, for example, experimenting with different cell culture conditions and feed strategies, to determine how product quality attributes are affected at different times in cell culture.

### Description of Terms

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. As used herein, the term polypeptide includes proteins, variants thereof, fragments thereof, and peptides, whether synthetic, naturally occurring, or derived from a larger polypeptide, for example through digestion or truncation. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like.

Proteins of interest or polypeptides of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference).

In some exemplary aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of: IgG, IgM, IgA1, IgA2, IgD, or IgE. In certain aspects the antibody molecule is a full-length antibody (e.g., an IgG1) or alternatively the antibody can be a fragment (e.g., an Fc fragment or a Fab fragment).

The term “antibody” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a Fc/2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen.

An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or KA-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the systems and methods disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some aspects, the protein of interest or polypeptide of interest is an antibody, a human antibody, a humanized antibody, a chimeric antibody, a monoclonal antibody, a multispecific antibody, a bispecific antibody, an antibody fragment, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, an IgG4 antibody, a fusion protein, a receptor fusion protein, an antibody-derived protein, or combinations thereof. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody.

In some aspects, the antibody is selected from the group consisting of an anti-Programmed Cell Death 1 antibody (e.g. an anti-PD1 antibody as described in U.S. Pat. App. Pub. No. US2015/0203579A1), an anti-Programmed Cell Death Ligand-1 antibody (e.g. an anti-PD-L1 antibody as described in in U.S. Pat. App. Pub. No. US2015/0203580A1), an anti-DII4 antibody, an anti-Angiopoetin-2 antibody (e.g. an anti-ANG2 antibody as described in U.S. Pat. No. 9,402,898), an anti-Angiopoetin-Like 3 antibody (e.g. an anti-AngPt13 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an anti-C5 antibody as described in U.S. Pat. App. Pub. No US2015/0313194A1), an anti-TNF antibody, an anti-epidermal growth factor receptor antibody (e.g. an anti-EGFR antibody as described in U.S. Pat. No. 9,132,192 or an anti-EGFRvIII antibody as described in U.S. Pat. App. Pub. No. US2015/0259423A1), an anti-Proprotein Convertase Subtilisin Kexin-9 antibody (e.g. an anti-PCSK9 antibody as described in U.S. Pat. No. 8,062,640 or U.S. Pat. App. Pub. No. US2014/0044730A1), an anti-Growth And Differentiation Factor-8 antibody (e.g. an anti-GDF8 antibody, also known as anti-myostatin antibody, as described in U.S. Pat. Nos. 8,871,209 or 9,260,515), an anti-Glucagon Receptor (e.g. anti-GCGR antibody as described in U.S. Pat. App. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. App. Pub. No. US2014/0271681A1 or U.S. Pat. Nos. 8,735,095 or 8,945,559), an anti-interleukin 6 receptor antibody (e.g. an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g. anti-IL33 antibody as described in U.S. Pat. App. Pub. Nos. US2014/0271658A1 or US2014/0271642A1), an anti-Cluster of differentiation 3 antibody (e.g. an anti-CD3 antibody, as described in U.S. Pat. App. Pub. Nos. US2014/0088295A1 and US20150266966A1, and in U.S. Application No. 62/222,605), an anti-Cluster of differentiation 20 antibody (e.g. an anti-CD20 antibody as described in U.S. Pat. App. Pub. Nos. US2014/0088295A1 and US20150266966A1, and in U.S. Pat. No. 7,879,984), an anti-CD19 antibody, an anti-CD28 antibody, an anti-Cluster of Differentiation-48 antibody (e.g. anti-CD48 antibody as described in U.S. Pat. No. 9,228,014), an anti-Fel dl antibody (e.g. as described in U.S. Pat. No. 9,079,948), an anti-influenza virus antibody, an anti-Respiratory syncytial virus antibody (e.g. anti-RSV antibody as described in U.S. Pat. App. Pub. No. US2014/0271653A1), an anti-Middle East Respiratory Syndrome virus antibody (e.g. an anti-MERS-COV antibody as described in U.S. Pat. App. Pub. No. US2015/0337029A1), an anti-Ebola virus antibody (e.g. as described in U.S. Pat. App. Pub. No. US2016/0215040), an anti-Zika virus antibody, an anti-Severe Acute Respiratory Syndrome (SARS) antibody (e.g., an anti-SARS-COV antibody), an anti-COVID-19 antibody (e.g., an anti-SARS-COV-2 antibody), an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. App. Pub. No. US2016/0017029 and U.S. Pat. Nos. 8,309,088 and 9,353,176) and an anti-Activin A antibody. In some aspects, the bispecific antibody is selected from the group consisting of an anti-CD3×anti-CD20 bispecific antibody (as described in U.S. Pat. App. Pub. Nos. US2014/0088295A1 and US20150266966A1), an anti-CD3×anti-Mucin 16 bispecific antibody (e.g., an anti-CD3×anti-Muc16 bispecific antibody), an anti-CD3×BCMA bispecific antibody, and an anti-CD3×anti-Prostate-specific membrane antigen bispecific antibody (e.g., an anti-CD3×anti-PSMA bispecific antibody). In one aspect, the protein of interest or polypeptide of interest comprises a combination of any of the foregoing.

In some aspects, the protein of interest or polypeptide of interest is selected from the group consisting of alirocumab, sarilumab, fasinumab, nesvacumab, dupilumab, trevogrumab, evinacumab, rinucumab, and modifications, truncations, and variations thereof. In one aspect, the protein of interest comprises a combination of any of the foregoing.

Dupilumab (DUPIXENT®) is a monoclonal antibody developed as a collaboration between Regeneron and Sanofi, approved in the United States by the Food and Drug Administration in March 2017 as the first antibody-based treatment of atopic dermatitis in adults (Thibodeaux et al., 2019, Hum. Vaccines & Immunother., 15:2129-2139; Rodrigues et al., 2019, G. Ital. Dermatol. Venereol., 154). Atopic dermatitis (AD) is a chronic inflammatory skin disorder affecting up to 20% of the worldwide population, characterized by xerotic, crythematous, and lichenified papules and plaques. Dupilumab can be used along with topical corticosteroids, or as the sole treatment (D′Ippolito and Pisano, 2018, Pharmacy and Therapeutics, 43 (9): 532).

In October 2018, DUPIXENT® was approved for the treatment of moderate-to-severe asthma with the eosinophilic phenotype for patients aged 12 and older as an add-on maintenance therapy aimed to suppress the atopic conditions and improve patient life quality by reducing symptoms and morbidity (Thibodeaux et al.). DUPIXENT® has also been approved for the treatment AD in children over 6 months old, asthma of the eosinophilic phenotype or when oral corticosteroid-dependent in children over 6 years old, eosinophilic esophagitis (EoE) in patients over the age of 12, prurigo nodularis (PN) in adults, and as an add-on treatment for chronic rhinosinusitis with nasal polyposis (CRSwNP) in adults (Patient Information-DUPIXENT® (dupilumab) injection for subcutaneous use, Regeneron, regeneron.com/downloads/dupixent_ppi.pdf, (accessed 25 May 2023); Take Action with DUPIXENT® (dupilumab), dupixent.com, (accessed 24 Jul. 2023)). Additionally, dupilumab is currently undergoing numerous clinical trials, including for new indications such as chronic obstructive pulmonary disease (COPD) (Pivotal Study to Assess the Efficacy, Safety and Tolerability of Dupilumab in Patients With Moderate to Severe COPD With Type 2 Inflammation ClinicalTrials.gov, classic.clinicaltrials.gov/ct2/show/NCT04456673, (accessed 24 Jul. 2023)), bullous pemphigoid (A Study to Evaluate the Efficacy and Safety of Dupilumab in Adult Patients With Bullous Pemphigoid-ClinicalTrials.gov, classic.clinicaltrials.gov/ct2/show/NCT04206553, (accessed 24 Jul. 2023)), chronic rhinosinusitis without nasal polyps (CRSsNP) (Dupilumab in CRSsNP-ClinicalTrials.gov, classic.clinicaltrials.gov/ct2/show/NCT04678856, (accessed 24 Jul. 2023)), keloids (Study to Assess Efficacy and Safety of Dupilumab in the Treatment of Keloids-ClinicalTrials.gov, classic.clinicaltrials.gov/ct2/show/NCT05128383, (accessed 24 Jul. 2023)), alopecia areata (Regeneron AA Multicenter (Dupilumab)-ClinicalTrials.gov, classic.clinicaltrials.gov/ct2/show/NCT05551793, (accessed 24 Jul. 2023)), chronic hepatic pruritus (Dupilumab for the Treatment of Moderate to Severe Chronic Hepatic Pruritus-ClinicalTrials.gov, classic.clinicaltrials.gov/ct2/show/NCT04256759, (accessed 24 Jul. 2023)), eosinophilic gastritis (Dupilumab in Eosinophilic Gastritis-ClinicalTrials.gov, classic.clinicaltrials.gov/ct2/show/NCT03678545, (accessed 24 Jul. 2023)), allergy to cow's milk (Dupilumab and Milk OIT for the Treatment of Cow's Milk Allergy-ClinicalTrials.gov, classic.clinicaltrials.gov/ct2/show/NCT04148352, (accessed 24 Jul. 2023)) and peanuts (Study in Pediatric Subjects With Peanut Allergy to Evaluate Efficacy and Safety of Dupilumab as Adjunct to AR101 (Peanut Oral Immunotherapy)-ClinicalTrials.gov, classic.clinicaltrials.gov/ct2/show/NCT03682770, (accessed 24 Jul. 2023)), and as a neoadjuvant therapy in men with high-risk prostate cancer (Neoadjuvant Dupilumab in Men With Localized High-Risk Prostate Cancer-ClinicalTrials.gov, classic.clinicaltrials.gov/ct2/show/NCT03886493, (accessed 24 Jul. 2023)).

Dupilumab is a fully human IgG4 monoclonal antibody with a molecular weight of approximately 147 kDa and is produced using Chinese hamster ovary (CHO) cell suspension culture (D′Ippolito and Pisano; Patient Information). The antibody binds to the IL-4Ra subunits of Type 1 and Type 2 IL-4 receptors, inhibiting the IL-4 and IL-13 signaling pathways. This reduces the release of cytokines and chemokines, which are inflammatory mediators, as well as the release of nitric oxide and IgE, and leads to an increase in IL-4 and IL-13 serum levels. IL-4 and IL-13 play a key role in type 2 inflammation, which is integral to atopic diseases such as asthma, atopic dermatitis, or chronic sinusitis with nasal polyposis. IL-4 induces naïve CD4+ T cells to differentiate into Th2 effector cells, and IL-13 is involved in goblet cell metaplasia, smooth muscle alterations, fibrosis, mucus hypersecretion, and increased airway hyperreactivity. Additionally, both IL-4 and IL-13 promote the chemotaxis of eosinophils to inflammation sites, and class switching of B-cell immunoglobulins to IgE and IgG4 (in humans) or IgG1 (in mice) (Thibodeaux et al.; Le Floc′h et al., 2020, Allergy, 75:1188-1204).

Dupilumab has been shown to decrease FeNO (fractional exhaled nitric oxide) and the circulating concentrations of total IgE, allergen-specific IgE, cotaxin-3, periostin, and chemokine CCL17 in asthma patients, relative to placebo (Patient Information). In the case of atopic dermatitis, dupilumab has been shown decrease the expression of genes involved in epidermal hyperplasia (MKi67 and K16) and the Th2 inflammatory response (IL-4, IL-14, CCL17, CCL18, CCL26), reducing the thickness of lesional skin, relative to placebo (Thibodeaux et al.). When used to treat chronic rhinosinusitis with nasal polyposis, dupilumab was found to reduce polyp size and the concentration of type 2 inflammation biomarkers (cotaxin-3 and total IgE) in blood, nasal secretions, and polyp tissues, as well as rapidly improve smell in CRSwNP patients, relative to placebo (Jonstam et al., 2019, Allergy, 74:743-752).

In some aspects, the protein of interest or polypeptide of interest is a recombinant protein that contains an Fc moiety and another domain, (e.g., an Fc-fusion protein). In some aspects, an Fc-fusion protein is a receptor Fc-fusion protein, which contains one or more extracellular domain(s) of a receptor coupled to an Fc moiety. In some aspects, the Fc moiety comprises a hinge region followed by a CH2 and CH3 domain of an IgG. In some aspects, the receptor Fc-fusion protein contains two or more distinct receptor chains that bind to either a single ligand or multiple ligands. For example, an Fc-fusion protein is a TRAP protein, such as for example an IL-1 trap (e.g., rilonacept, which contains the IL-IRAcP ligand binding region fused to the II-1R1 extracellular region fused to Fc of hIgG1; see U.S. Pat. No. 6,927,004, which is herein incorporated by reference in its entirety), or a VEGF trap (e.g., aflibercept or ziv-aflibercept, which contains the Ig domain 2 of the VEGF receptor Fltl fused to the Ig domain 3 of the VEGF receptor Flk1 fused to Fc of hIgG1; see U.S. Pat. Nos. 7,087,411 and 7,279,159). In other aspects, an Fc-fusion protein is a ScFv-Fc-fusion protein, which contains one or more antigen-binding domain(s), such as a variable heavy chain fragment and a variable light chain fragment, of an antibody coupled to an Fc moiety.

The following identifies and describes proteins made in cell culture that can be produced, used, or characterized according to the present inventions. Cells comprising the requisite DNA encoding these proteins can be cultured for production according to the present inventions.

For example, for antibody production, the inventions are amenable for research and production use for diagnostics and therapeutics based upon all major antibody classes, namely IgG, IgA, IgM, IgD and IgE. IgG is a preferred class, and includes subclasses IgG1 (including IgG12 and IgG1K), IgG2, IgG3, and IgG4. Further antibody embodiments include a human antibody, a humanized antibody, a chimeric antibody, a monoclonal antibody, a multispecific antibody, a bispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In one embodiment, the antibody is an IgG2 antibody. In one embodiment, the antibody is an IgG4 antibody. In one embodiment, the antibody is a chimeric IgG2/IgG4 antibody. In one embodiment, the antibody is a chimeric IgG2/IgG1 antibody. In one embodiment, the antibody is a chimeric IgG2/IgG1/IgG4 antibody. Derivatives, components, domains, chains and fragments of the above also are included.

Further antibody embodiments include a human antibody, a humanized antibody, a chimeric antibody, a monoclonal antibody, a multispecific antibody, a bispecific antibody, a trispecific antibody, an antigen binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, or an IgG4 antibody. In one embodiment, the antibody is an IgG1 antibody. In an embodiment, the antibody is an IgG2 antibody. In one embodiment, the antibody is an IgG4 antibody. In another embodiment, the antibody is a chimeric IgG2/IgG4 antibody. In another embodiment, the antibody is a chimeric IgG2/IgG1 antibody. In another embodiment, the antibody is a chimeric IgG2/IgG1/IgG4 antibody.

In additional embodiments, the antibody is selected from the group consisting of an anti-Programmed Cell Death 1 antibody (for example an anti-PD1 antibody as described in U.S. Pat. Appln. Pub. No. US2015/0203579A1), an anti-Programmed Cell Death Ligand-1 (for example an anti-PD-L1 antibody as described in in U.S. Pat. Appln. Pub. No. US2015/0203580A1), an anti-DI14 antibody, an anti-Angiopoetin-2 antibody (for example an anti-ANG2 antibody as described in U.S. Pat. No. 9,402,898), an anti-Angiopoetin-Like 3 antibody (for example an anti-AngPt13 antibody as described in U.S. Pat. No. 9,018,356), an anti-platelet derived growth factor receptor antibody (for example an anti-PDGFR antibody as described in U.S. Pat. No. 9,265,827), an anti-Erb3 antibody, an anti-Prolactin Receptor antibody (for example anti-PRLR antibody as described in U.S. Pat. No. 9,302,015), an anti-Complement 5 antibody (for example an 25 anti-C5 antibody as described in U.S. Pat. Appln. Pub. No US2015/0313194A1), an anti-TNF antibody, an anti-epidermal growth factor receptor antibody (for example an anti-EGFR antibody as described in U.S. Pat. No. 9,132,192 or an anti-EGFRvIII antibody as described in U.S. Pat. Appln. Pub. No. US2015/0259423A1), an anti-Proprotein Convertase Subtilisin Kexin-9 antibody (for example an anti-PCSK9 antibody as described in U.S. Pat. No. 8,062,640 or U.S. Pat. Appln. Pub. No. US2014/0044730A1), an anti-Growth And Differentiation Factor-8 antibody (for example an anti-GDF8 antibody, also known as anti-myostatin antibody, as described in U.S. Pat. Nos. 8,871,209 or 9,260,515), an anti-Glucagon Receptor (for example anti-GCGR antibody as described in U.S. Pat. Appln. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. Appln. Pub. No. US2014/0271681A1 or U.S. Pat. Nos. 8,735,095 or 8,945,559), an anti-interleukin 6 receptor antibody (for example an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (for example anti-IL33 antibody as described in U.S. Pat. Appln. Pub. Nos. US2014/0271658A1 or US2014/0271642A1), an anti-Respiratory syncytial virus antibody (for example anti-RSV antibody as described in U.S. Pat. Appln. Pub. No. US2014/0271653A1), an anti-Cluster of differentiation 3 (for example an anti-CD3 antibody, as described in U.S. Pat. Appln. Pub. Nos. US2014/0088295A1 and US20150266966A1, and in U.S. Application No. 62/222,605), an anti-Cluster of differentiation 20 (for example an anti-CD20 antibody as described in U.S. Pat. Appln. Pub. Nos. US2014/0088295A1 and US20150266966A1, and in U.S. Pat. No. 7,879,984), an anti-CD19 antibody, an anti-CD28 antibody, an anti-Cluster of Differentiation 48 (for example anti-CD48 antibody as described in U.S. Pat. No. 9,228,014), an anti-Fel dl antibody (for example as described in U.S. Pat. No. 9,079,948), an anti-Middle East Respiratory Syndrome virus (for example an anti-MERS antibody as described in U.S. Pat. Appln. Pub. No. US2015/0337029A1), an anti-Ebola virus antibody (for example as described in U.S. Pat. Appln. Pub. No. US2016/0215040), an anti-Zika virus antibody, an anti-Lymphocyte Activation Gene 3 antibody (for example an anti-LAG3 antibody, or an anti-CD223 antibody), an anti-Nerve Growth Factor antibody (for example an anti-NGF antibody as described in U.S. Pat. Appln. Pub. No. US2016/0017029 and U.S. Pat. Nos. 8,309,088 and 9,353,176) and an anti-Activin A antibody. In some embodiments, the bispecific antibody is selected from the group consisting of an anti-CD3×anti-CD20 bispecific antibody (as described in U.S. Pat. Appln. Pub. Nos. US2014/0088295A1 and US20150266966A1), an anti-CD3× anti-Mucin 16 bispecific antibody (for example, an anti-CD3×anti-Muc16 bispecific antibody), and an anti-CD3×anti-Prostate-specific membrane antigen bispecific antibody (for example, an anti-CD3×anti-PSMA bispecific antibody). See also U.S. Patent Publication No. US 2019/0285580 A1. Also included are a Met×Met antibody, an agonist antibody to NPR1, an LEPR agonist antibody, a BCMA×CD3 antibody, a MUC16×CD28 antibody, a GITR antibody, an IL-2Rg antibody, an EGFR×CD28 antibody, a Factor XI antibody, antibodies against SARS-CoC-2 variants, a Fel d 1 multi-antibody therapy, a Bet v 1 multi-antibody therapy. Derivatives, components, domains, chains and fragments of the above also are included.

Cells that produce exemplary antibodies can be cultured, used, or characterized according to the inventions. Exemplary antibodies include alirocumab, atoltivimab, maftivimab, odesivimab, odesivivmab-ebgn, casirivimab, imdevimab, cemiplimab and cemiplimab-rwlc (human IgG4 monoclonal antibody that binds PD-1), dupilumab (human monoclonal antibody of the IgG4 subclass that binds to the IL-4R alpha (a) subunit and thereby inhibits interleukin 4 (IL-4) and Interleukin 13 (IL-13) signalling), evinacumab, evinacumab-dgnb, fasinumab, fianlimab, garetosmab, itepekimab nesvacumab, odrononextamab, pozelimab, sarilumab, trevogrumab, and rinucumab.

Additional exemplary antibodies include ravulizumab-cwvz, abciximab, adalimumab, adalimumab-atto, ado-trastuzumab, alemtuzumab, atezolizumab, avelumab, basiliximab, belimumab, benralizumab, bevacizumab, bezlotoxumab, blinatumomab, brentuximab vedotin, brodalumab, canakinumab, capromab pendetide, certolizumab pegol, cetuximab, denosumab, dinutuximab, durvalumab, cculizumab, clotuzumab, emicizumab-kxwh, emtansine alirocumab, evolocumab, golimumab, guselkumab, ibritumomab tiuxetan, idarucizumab, infliximab, infliximab-abda, infliximab-dyyb, ipilimumab, ixekizumab, mepolizumab, necitumumab, nivolumab, obiltoxaximab, obinutuzumab, ocrelizumab, ofatumumab, olaratumab, omalizumab, panitumumab, pembrolizumab, pertuzumab, ramucirumab, ranibizumab, raxibacumab, reslizumab, rinucumab, rituximab, secukinumab, siltuximab, tocilizumab, trastuzumab, ustekinumab, and vedolizumab.

In addition to next generation products, the inventions also are applicable to production and/or characterization of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries.

### Recombinant Protein Production

As used herein, a “sample” can be obtained from any step of a bioprocess, such as cell culture fluid (CCF), harvested cell culture fluid (HCCF), any step in the downstream processing, final concentrated pool (FCP), drug substance (DS), or a drug product (DP) comprising the final formulated product. In some aspects, a sample may be obtained from a cell culture at day 3, day 4, day 5, day 6, day 7, day 8, day 9, day 10, day 11, day 12, day 13, day 14, and/or day 15 of the cell culture. In some aspects, a sample may be obtained from a culturing vessel, for example, a plate, a flask, a bag, or a bioreactor. In some aspects, the culturing vessel may have a volume of, for example, 250 mL, 500 mL, 1 L, 2 L, 5 L, 10 L, 50 L, 500 L, 1,000 L, 2,000 L, 3,000 L, 5,000 L, 10,000 L, 15,000 L, 20,000 L, or 25,000 L.

In some specific aspects, the sample can be selected from any step of the downstream process of clarification, chromatographic production, or filtration. In some specific exemplary aspects, the drug product can be selected from manufactured drug product in the clinic, shipping, storage, or handling.

In some exemplary aspects, the protein of interest or polypeptide of interest can be produced from mammalian cells. The mammalian cells can be of human origin or non-human origin, and can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK (15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSAl cells, Swiss/3T3 cells, Indian muntjac cells, SIRC cells, Cn cells, and Jensen cells, Sp2/0, NS0, NS1 cells or derivatives thereof).

A recombinant protein of interest, for example an antibody, may be isolated from a cell culture medium, serum, plasma, ascitic fluid or a bacterial culture medium, and require multiple purification steps to separate out any contaminants related to the product, manufacturing process or the host cells (Rathore and Bhambure, 2014, Methods in Molecular Biology, 29-37).

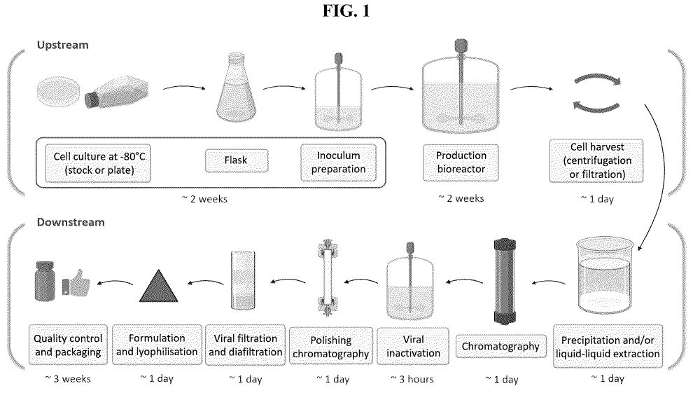

Large-scale antibody drug manufacturing generally utilizes a cell culture medium created from a master cell bank, a number of seed and production bioreactors, followed by cell removal steps, antibody purification (typically including affinity chromatography), viral inactivation (using detergents or low pH), polishing steps, and viral filtration. The product is then concentrated, diafiltered, and formulated (Chahar et al., 2020, Biologicals, 63:1-13; Jin et al., 2019, mAbs, 11:1479-1491). An exemplary workflow of a mAb production process is shown in FIG. 1.

The upstream process generally includes cultivating mammalian cells (e.g. CHO cells) genetically modified to produce antibodies of interest. Cells frozen in a cell bank are thawed and cultured. They are subsequently grown in progressively larger volumes over about two weeks' time, in order to create a seed culture for the bioreactor tanks.

In the biorcactors (with volumes up to, for example, 20,000 liters (Vázquez-Rey and Lang, 2011, Biotechnol. Bioeng., 108:1494-1508)), optimal conditions are maintained for the cells to grow and produce proteins. This includes providing the necessary metabolic substrates and growth factors, as well as controlling the temperature, pH, dissolved oxygen and other gases, while maintaining the environment to be free of microbial contamination (Jozala et al., 2016, Braz. J. Microbiol., 47:51-63). These conditions affect the synthesis of the antibody of interest and its potential degradation throughout the upstream manufacturing process, and are one of the determining factors for the quality profile of the produced drug (Das et al., 2020, J. Pharm. Sci., 109:116-133).

When the production phase is finished, typically either microfiltration or centrifugation is employed to separate out the cells, and the harvested cell culture supernatant is used in the downstream process to recover the biopharmaceutical product (Vázquez-Rey and Lang).

The upstream processing phase of recombinant protein production is followed by downstream processing. The purpose of downstream processing is to separate proteins from other components of the cell culture mixture, as well as to separate the antibody of interest from other proteins without losing its chemical integrity and biological activity. Usually, a sequence of at least two or three different separation steps is employed (Rathore and Bhambure, 2014), and they can be based on solubility, hydrophobicity, density, charge and charge distribution, isoelectric point, ligand-binding affinity, reversible associations, metal binding, posttranslational modifications, size, or shape. The initial purification steps commonly consist of lower-resolution higher-capacity steps, while the later steps are often higher-resolution lower-capacity, to account for the decrease in protein content throughout the purification process (Labrou, 2014, Methods in Molecular Biology, 3-10).

The most common purification technique used for monoclonal antibodies is affinity chromatography (Arora et al., 2014, Methods in Molecular Biology, 497-516), and Protein A (ProA) affinity chromatography specifically is usually the first step of the downstream process due to its high reliability, stability, and reproducibility (dos Santos et al., 2017, Biotechnol. Adv., 35:41-50). That step is often followed by viral inactivation (VI), since both VI and elution of antibodies from the Protein A resin require low-pH conditions. Polishing steps following viral inactivation typically involve ion-exchange chromatography (IEC) and membrane filtration (Vázquez-Rcy and Lang). The product can then undergo formulation and packaging. The resulting antibody product should be free of contaminants such as host cells and their proteins, nucleic acids, viruses, pyrogens, leachates, and cell culture media components, as well as undesired protein isoforms that could arise due to posttranslational modifications (Labrou).

While proteins produced for some purposes may be used in a crude state, the biopharmaceutical industry and the stringent drug product quality regulations it abides by require exceptionally high purity, emphasizing the importance of robust purification procedures to ensure the safety, identity, strength, purity and quality of the biotherapeutic delivered to the patient, and significantly increasing the costs. Downstream processing is estimated to make up 50-80% of protein manufacturing costs, with chromatography contributing up to 60% of the downstream costs (Labrou; Bracewell et al., 2015, BioPharm International, 28 (3)).

Protein A chromatography is often regarded as a “bottleneck”, as it not only uses particularly expensive resins, but it can also require multiple cycles for every batch and possesses capacity and diffusion limitations (dos Santos). As the upstream production process has become more and more efficient in the last few decades, the downstream process has become the limiting factor due to the inability to purify increasing volumes of materials at the same rate. It is therefore crucial to maximize the yield and minimize the number of separation steps performed when developing downstream purification procedures (Shi and Sun, 2020, Chin. J. Chem. Eng., 30:194-203; Labrou).

Chromatographic techniques have been improved through advancements in resin properties, such as optimization of pore structure, size, and volume to enhance the dynamic binding capacity, and ligand chemistry, such as immobilization on beads, which improves accessibility of ligands (Rathore et al., 2018, Biotechnol. Lett., 40:895-905). However, the development of chromatographic purification steps faces many challenges, including poor understanding of the protein-related processes, poor characterization of the raw materials or feed material, product instability, and low feed concentration (Rathore and Bhambure).

Methods alternative to chromatography, better suited for high volumes produced by the upstream process on an industrial scale, have also been explored for protein purification, including liquid-liquid phase extraction, membrane processes, precipitation and crystallization, and magnetic separation (dos Santos). Nevertheless, despite its limitations, chromatography (particularly ProA chromatography) remains the most common technique used in downstream processing (Rathore et al., 2018), due to its high resolution of complex mixtures and high selectivity for biotherapeutic molecules (Labrou).

In some aspects, a sample can be prepared prior to or following enrichment steps, separation steps, and/or analysis steps. Preparation steps can include alkylation, reduction, denaturation, digestion, derivatization, and/or deglycosylation.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl) phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some aspects, non-reduced preparation may be used, for example, in order to preserve an endogenous disulfide bond between Fab arms of an antibody or antibody-derived protein. In other aspects, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein or polypeptide. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein or polypeptide. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C), outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

In some exemplary aspects, IdeS or a variant thereof is used to cleave an antibody below the hinge region, producing an Fc fragment and a Fab₂ fragment. Digestion of an analyte may be advantageous because size reduction may increase the sensitivity and specificity of characterization and detection of the analyte using LC-MS. When used for this purpose, digestion that separates out an Fc fragment and keeps a Fab₂ fragment for analysis may be preferred. This is because variable regions of interest, such as the complementarity-determining region (CDR) of an antibody, are contained in the Fab₂ fragment, while the Fc fragment may be relatively uniform between antibodies and thus provide less relevant information. Alternatively, or additionally, digestion that separates out a Fab₂ fragment and keeps an Fc fragment for analysis may be preferred, because the Fc fragment contains an N-glycosylation site of interest.

IdeS digestion has a high efficiency, allowing for high recovery of an analyte. The digestion and elution process may be performed under native conditions, allowing for simple coupling to a native LC-MS system. IdeS or variants thereof are commercially available and may be marketed as, for example, FabRICATOR® or FabRICATOR Z®.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC). In some aspects, a sample can be subjected to any one of the aforementioned chromatographic methods or a combination thereof. Analytes separated using chromatography will feature distinctive retention times, reflecting the speed at which an analyte moves through the chromatographic column. Analytes may be compared using a chromatogram, which plots retention time on one axis and measured signal on another axis, where the measured signal may be produced from, for example, UV detection or fluorescence detection.

In some exemplary aspects, the methods and systems of the present invention may include subjecting a sample to affinity chromatography. Affinity chromatography is an analytical technique based on the specific and reversible interactions between proteins and their ligands (e.g. hormone and receptor, enzyme and substrate, or antibody and its target antigen), allowing for the selective binding and separation of target proteins from complex mixtures. Affinity chromatography columns usually consist of ligands covalently immobilized on a solid support such as sepharose or agarose. When a sample is passed through the column, the target protein binds to the affinity ligands, and unbound or weakly bound sample components are removed in a washing step. The target protein can then be eluted by changing factors such as polarity, ionic strength, or pH, or by adding denaturing agents or competitive protein analogues to the column (Arora et al., 2017, Methods, 116:84-94; Labrou; Arora et al., 2014; Urh et al., 2009, Methods in Enzymology, 417-438).

Affinity chromatography can involve subjecting a biological sample to a column comprising a suitable Protein A resin. When used herein, the term “Protein A” encompasses Protein A recovered from a native source thereof, Protein A produced synthetically (e.g., by peptide synthesis or by recombinant techniques), and variants thereof which retain the ability to bind proteins which have a CH2/CH3 region. Protein A may additionally bind to human IgG molecules containing IgG F(ab′)2 fragments from the human VH3 gene family (Roben et al., J Immunol., 1995, 154 (12): 6437-45). In certain aspects, Protein A resin is useful for affinity-based production and isolation of a variety of antibody isotypes by interacting specifically with the Fc portion of a molecule, should it possess that region.

In Protein A affinity chromatography, the ProA ligand is a single polypeptide chain with molecular weight of ˜46.8 kDa, derived either from Staphylococcus aureus or from Escherichia coli. Protein A contains the homologous domains E, D, A, B, and C, which can bind to the Fc region between the CH2 and CH3 (constant heavy 2 and 3) domains of IgG class of antibodies. It can also interact with the variable region between the complementary determining regions CDR2 and CDR3 in the heavy chain, and with polyclonal antibodies (Shi and Sun; Arora et al., 2014; Urh et al.; Ramos-de-la-Peña et al., 2019, J. Sep. Sci., 42:1816-1827).

There are several commercial sources for Protein A resin. Suitable resins include, but are not limited to, MabSelect PrismA, MabSelect SuRe, MabSelect SuRe LX, MabSelect, MabSelect SuRe pcc, MabSelect Xtra, rProtein A Sepharose from Cytiva, ProSep HC, ProSep Ultra, ProSep Ultra Plus from EMD Millipore, MabCapture from ThermoFisher, and Amsphere™ A3 from JSR Life Sciences.

Another commonly used ligand, protein G, is derived from group C and G Streptococci (Ramos-de-la-Peña et al.), and has the molecular weight of ˜21.6 kDa. Protein G is usually less preferable than Protein A because it can bind to albumin, kininogen, and α 2-macroglobulin, resulting in a decreased antibody purification efficiency. Additionally, protein G has a lower binding capacity and is less stable in acidic conditions (required for the elution step) (Arora et al., 2014). Other proteins which have been used for antibody purification include protein B (also derived from Streptococci) which binds human IgA, and protein L (derived from Peptostreptococcus magnus) which binds to the light chains of the Fab regions, making it especially useful for purifying antibodies which lack Fc regions (Chahar et al.; Urh et al.).

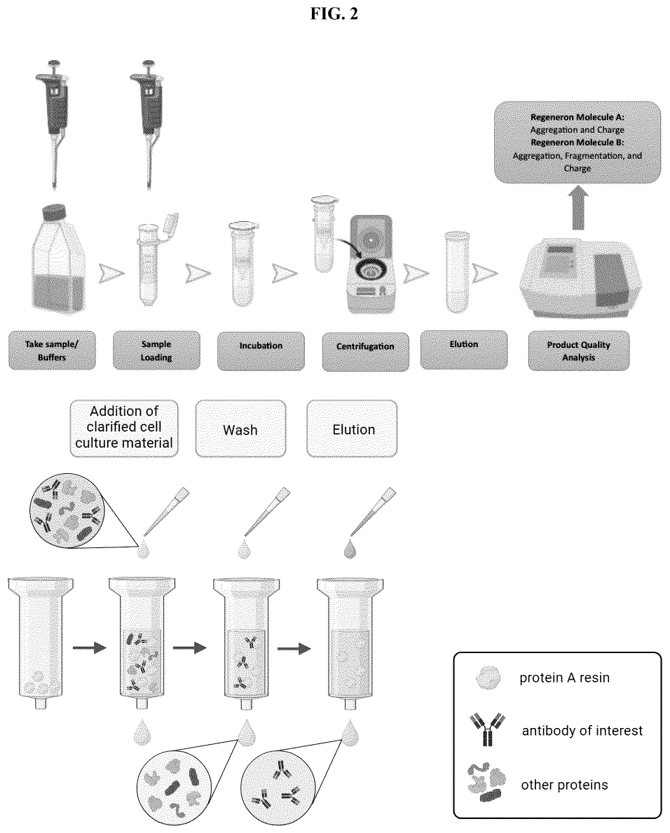

Preparation of samples for antibody purification using Protein A affinity chromatography usually involves clarifying cell culture, which comprises removing the bulk of insoluble components such as cells by centrifugation, filtration, or precipitation using ammonium sulphate, caprylic acid, or polyethylene glycol (PEG) (Arora et al., 2017; Arora et al., 2014). Target antibodies selectively bind to the Protein A ligands while other sample components (including host cell proteins, nucleic acids, medium components, product isoforms and fragments) pass through the resin (Ramos-de-la-Peña et al.). Optimal binding usually occurs near neutral pH, i.e. 8.2 (similarly, pH 7-7.5 for protein G chromatography, and pH 7.5 for protein L chromatography). The resin is washed to remove any remaining unbound or weakly-bound antibodies and impurities such as nucleic acids or host cell proteins. The bound antibodies are then eluted by lowering the pH (typically to 2.5-4) and consequently weakening the interactions between them and the protein ligands. In antibody manufacturing, the viral inactivation step is then performed, before the eluate is neutralized to improve stability of the proteins and minimize or prevent denaturation, aggregation, and loss of biological activity (Chahar et al.; Urh et al.; Zhang et al., 2019, Protein Expr. Purif., 158:65-73). An exemplary workflow of Protein A chromatography using a centrifugal (spin) column is shown in FIG. 2.

Despite being the method of choice for antibody purification, Protein A affinity chromatography has significant limitations, including relatively low binding capacity, high cost of the Protein A resin (nearly 50% higher than the cost of resins used in traditional chromatography), and generating additional impurities due to ligands co-eluting in fragments called Protein A leachate (Bracewell et al.; Katcia et al., 2018, J. Chromatogr. A, 1579:60-72). Moreover, affinity chromatography is not very effective at removing aggregates, as those can bind more strongly than monomer antibodies but can still elute in the pH range used in the elution step, and can even be formed at the low elution pH (Yu et al., 2016, J. Chromatogr. A, 1457:66-75; Amritkar et al., 2020, Biotechnol. Adv., 44:107632). It is therefore important to implement polishing steps (e.g. ion exchange chromatography (IEC) (Ramos-de-la-Peña et al.)) able to remove aggregates and other impurities in the downstream process (Zhang et al., 2019).

Additionally, in some manufacturing plants, each batch is cycled multiple times using a smaller Protein A column rather than as a single step using a large column. Consequently, the processing time is prolonged, the throughput decreases, and the intensive re-using of columns can lead to resin damage, degradation and leaching of ligands, and a reduced available surface area (Bracewell et al.).

Ligands alternative to Protein A have been investigated, including aptamers, short peptides, affitins, affibodies, or ankyrin repeat proteins, but Protein A affinity chromatography remains the gold standard of mAb purification, as it delivers high recovery (>95%) and high purity (from 95% to >99%) due to a high binding affinity for the target protein (Curling, 2017, Process Scale Purification of Antibodies, 23-54; Ramos-de-la-Peña et al.). Moreover, affinity chromatography may be used to separate active and inactive forms of proteins, or to concentrate low-concentration samples (Urh et al.). Protein A affinity chromatography is thus used both in the purification of antibodies during manufacturing and as an analytical tool (Rodriguez et al., 2020, J. Chromatogr., 1157:122332).

In some aspects, an affinity column may be a centrifugal affinity column (or a “spin column”), wherein bound or unbound components can be removed from the column using centrifugation. In some aspects, the use of a centrifugal column may have the advantage of being capable of processing small volume samples compared to a conventional flow-based column, and being capable of processing multiple samples simultaneously.

An affinity column can be equilibrated with a suitable buffer prior to sample loading. A pH of a Protein A load may be, for example, between about 6 and about 8, between about 6 and about 7, between about 7 and about 8, or about 6. Following loading of the column, the column can be washed one or multiple times using a suitable wash buffer. The column can then be eluted using an appropriate elution buffer, for example, glycine-HCl, acetic acid, or citric acid. The eluate can be monitored using techniques well known to those skilled in the art such as a UV detector. The eluted fractions of interest can be collected and then prepared for further processing.

A Protein A wash buffer may be selected on the basis of its ability to disrupt protein-protein interactions, for example interactions between a protein of interest and impurities such as HCPs, without disrupting interactions between the protein of interest and the chromatographic material. Suitable wash buffers for HCP removal may comprise, for example, salts (e.g. sodium-containing salts such as sodium phosphate or sodium chloride, potassium-containing salts such as potassium sorbate, magnesium-containing salts, hydrochloride-containing salts such as guanidine hydrochloride, Tris-containing salts), surfactants (e.g. polysorbate 20, polysorbate 80), polar materials (e.g. isopropanol, ethanol), or amino acids (e.g. arginine). Suitable wash buffers may have a pH between about 5 and about 9, about 5, about 5.5, about 6, about 6.5, about 7, about 7.5, about 8, about 8.5, or about 9.

### Product Quality Attributes

Identifying the critical Product Quality (cPQ) attributes of a biopharmaceutical is key to ensuring the efficacy and safety of the product. A product quality attribute may also be referred to as a PQA, and a critical quality attribute may be referred to as a CQA. According to the ICH Q8 (R2) Scientific Guideline for Pharmaceutical Development, a critical quality attribute is “a physical, chemical, biological, or microbiological property or characteristic that should be within an appropriate limit, range, or distribution to ensure the desired product quality” (ICH guideline Q8 (R2) on pharmaceutical development, European Medicines Agency, cma.europa.cu/en/documents/scientific-guideline/international-conference-harmonisation-technical-requirements-registration-pharmaceuticals-human-use_en-11.pdf (accessed 16 Jun. 2023)). Understanding the cPQ attributes and analyzing their variability is necessary to define the acceptance criteria and the quality target product profile (QTPP) (Alt et al., 2016, Biologicals, 44:291-305), and understanding how the various process parameters affect cPQ allows the development of the product with a quality by design (QbD) approach (Reusch and Tejada, 2015, Glycobiology, 25:1325-1334).

cPQ attributes can affect the product purity, stability, strength, and drug release, as well as other aspects specific to the formulation type, such as adhesion properties of patches, the sterility of products administered parenterally, or the aerodynamic properties of inhaled drugs (ICH guideline).

Assessing the cPQ includes detecting and quantifying not only process-related impurities but also aggregates, fragments, and product variants, commonly including charge variants (basic or acidic), size variants, oxidation-related variants, structural variants, and variants arising from glycosylation of the Fc region of antibodies (Alt et al.). Examples of such monoclonal antibody variants are listed in Table 1 below.

TABLE 1
Examples of biopharmaceutical product variants.

| Variant type | Example |
|---|---|
| Charge (basic) | Species with isomerized aspartic acid in |
| | complementarity determining regions |
| Charge (acidic) | Deamidated species |
| Size | HMW species; LMW species |
| Oxidation-related | Species with oxidized methionine |
| Structural | Sequence variants |
| Glycosylation-related | Afucosylated species |

A risk assessment can be performed to analyze the cPQ attributes and process parameters in terms of their impact on safety, pharmacokinetics (PK), bioactivity, and immunogenicity. This is usually done at an early stage of the development process based on initial experiments and prior knowledge, and the assessment is refined at a later stage, incorporating more experimental data and mathematical models (ICH guideline; Alt et al.). Impact scores may be used to better define vague terms such as “high impact” or “low impact” using point scales (Alt et al.). Non-limiting of examples of assays suitable for assessing product quality attributes include chromatography (including RPLC, IEX, AEX, CEX, SEC, HIC, HILIC, and MMC), mass spectrometry (including intact mass analysis, peptide mapping, and amino acid sequencing), spectroscopy (including UV/vis spectroscopy), capillary electrophoresis (including free flow electrophoresis, isoelectric focusing, capillary isoelectric focusing, imaged capillary isoelectric focusing, and capillary zone electrophoresis), and gel electrophoresis (including SDS-PAGE and western blotting), and ligand binding assays (including biolayer interferometry, enzyme-linked immunosorbent assay, and surface plasmon resonance).

Aggregation is one of the most common cPQ-related problems in biopharmaceutical manufacturing, as it can lead to loss of protein activity and product yield, purity, and stability (Jing et al., 2012, Process Biochem., 47:69-75; Torkashvand and Vaziri, 2017, Iran. Biomed. J., 21:131-141). It is also suspected that large aggregates can cause an adverse immune response in patients, such as anaphylaxis. Such large aggregates are hypothesized to be mistaken for the bacterial cell wall by the immune system, leading to immunogenicity. However, due to the complexity of the phenomenon and the contributing factors related to both the biopharmaceutical and the patient, the cause of immune responses cannot be conclusively attributed to aggregation only. Those opposing this hypothesis argue that the quantity of the administered aggregates is too small to be immunogenic (Eon-Duval et al., 2012, Biotechnol. Prog., 28:608-622).

Aggregates can be divided into different categories based on their properties, as summarized in Table 2.

TABLE 2
Protein aggregate classification.

| Property | Categories |
|---|---|
| Solubility | soluble/insoluble |
| Bonding | covalent/non-covalent |
| Interaction reversibility | reversible/non-reversible |
| Size | small dimers/large visible particles |
| Denaturation | native/denatured |

Aggregation can result from mechanical stress (shaking, stirring, pumping) or physicochemical stress, including changes in pH and osmolality of the cell culture medium, as well as changes in temperature, oxygen concentration, protein concentration, and exposure to air or metal surfaces (Jin et al.; Torkashvand and Vaziri; Eon-Duval et al.; Cromwell et al., 2006, AAPS J., 8:E572-E579). Aggregates can therefore form at any step of the manufacturing process, formulation, or during storage (Eon-Duval et al.). In the cell culture medium, aggregation can occur both within the cell after protein expression and following secretion into the medium (Vázquez-Rey and Lang).

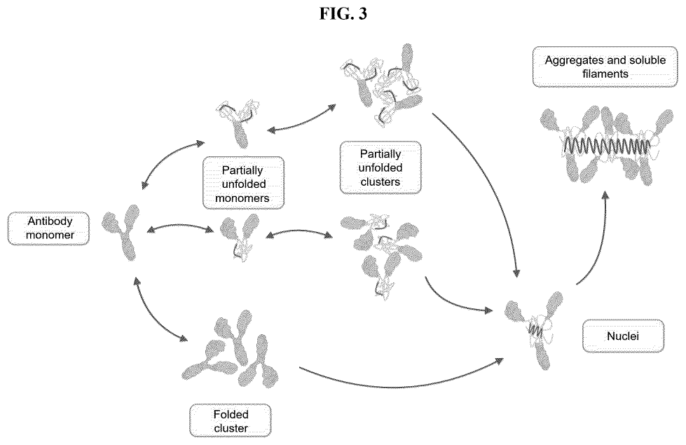

The mechanism of aggregation is not fully understood, but it is suspected that monomers with a modified secondary or tertiary structure act as precursors or intermediates of aggregation, as illustrated in FIG. 3 (Jefferis, 2018, Generics Biosimilars Initiat. J., 7:63-69). The altered structures expose hydrophobic regions which are shielded in unaltered proteins, potentially leading to aggregate formation (Jin et al.).

Aggregates arising during the upstream process can be removed in the downstream purification steps. While Protein A affinity chromatography is not used for aggregate removal, as aggregates may bind to the resin along with the monomer antibodies (Vázquez-Rey and Lang), other techniques, such as size-exclusion chromatography, cation- and anion-exchange chromatography, or ultrafiltration (in the case of insoluble aggregates), may be used (Cromwell et al.). However, this can lead to a reduction in yield; therefore, minimizing aggregation to begin with is more efficient (Jing et al.).

Aggregates can also form in the downstream process, particularly during the elution of antibodies captured by the Protein A resin, and during the viral inactivation step, both of which require acidic conditions (Vázquez-Rey and Lang; Cromwell et al.). Moreover, poor mixing during the VI step may lead to the formation of zones with pH lower than the intended VI conditions (Jin et al.).

The pH of the medium influences the charge distribution on the surface of the antibodies, and thus the inter- and intra-molecular interactions. Strong electrostatic interactions have been found to lead to proteins unfolding, meaning that ionic strength is also an important factor. Antibodies can be destabilized when high ionic strength screens their surface charges, particularly at low pH (Jin et al.).

Aggregation can increase when cell culture medium temperature is increased for an extended period of time (Cromwell et al.). Aggregate levels have also been shown to increase with increasing protein concentration due to a higher probability of interactions between proteins (Jin et al.).

Removing aggregates is more challenging compared to other impurities due to the biophysical similarities between the aggregates and the monomers. Their removal is usually mostly reliant on the polishing step, as affinity chromatography is not effective (Yu et al.).

Providing optimal conditions for the cell culture medium, including the feeding strategy, conductivity, osmolality, temperature, and pH, can help minimize the level of aggregation. Osmolytes such as amino acids, sugars, and polyols, which stabilize the proteins, are commonly added for aggregate level reduction (Torkashvand and Vaziri).

The presence of reducing and oxidizing substances containing bivalent copper ions, cysteine, and cystine have been found to decrease protein aggregation (Jing et al.). Another study found that a decrease in aggregation can be achieved by the addition of sodium chloride (Ju et al., 2009, J. Biotechnol., 143:145-150).

Other ways of reducing aggregation rates include using tank systems designed to minimize splashing and pumping, rapid freezing of bulk solution and the addition of cryo-protectors, as well as eliminating headspace in storage containers (Vázquez-Rey and Lang).

Aggregates can be measured using a variety of techniques, including sodium dodecyl sulfate polyacrylamide gel electrophoresis (SDS-PAGE), fluorescence spectroscopy, multi-angle laser light scattering (MALLS), and size-exclusion chromatography (SEC). SEC is the standard method in the biopharmaceutical industry, given its ability to measure aggregates both qualitatively and quantitatively, and the minimized impact on the environment and the protein structure possible due to the mild mobile phases employed (Fekete et al., 2014, J. Pharm. Biomed. Anal., 101:161-173).

Unlike other chromatographic techniques, SEC separates analytes based on their hydrodynamic radius through entropic processes, rather than their chemical properties and adsorption to stationary phase. SEC columns contain porous particles with controlled pore size distribution, which can be permeated by smaller molecules. The largest molecules (in this case oligomers) excluded from the pores clute first, followed by smaller molecules (dimers, monomers, antibody fragments) (Brusotti et al., 2017, Chromatographia, 81:3-23).

Analytes eluting from an SEC column may be separated into fractions based on elution time. For example, analytes eluting earlier than the functional form of a protein of interest, for example the monomeric form, may be broadly categorized as high molecular weight (HMW) species. A HMW fraction may be further subdivided into, for example, a very high molecular weight (vHMW) fraction and a dimer fraction (representing the elution time of a dimer of the protein of interest). Analytes eluting later than the functional form of a protein of interest may be broadly categorized as low molecular weight (LMW) species, and may be further subdivided into a LMW fraction and a later tail fraction. Variants of a protein of interest that have a higher molecular weight or lower molecular weight than the main species, or than the intended product, may be referred to as “size variants.”

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography may be made of dextrose, agarose, polyacrylamide, or silica which have different physical characteristics. Polymer combinations can also be also used. Most commonly used are those under the tradename “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

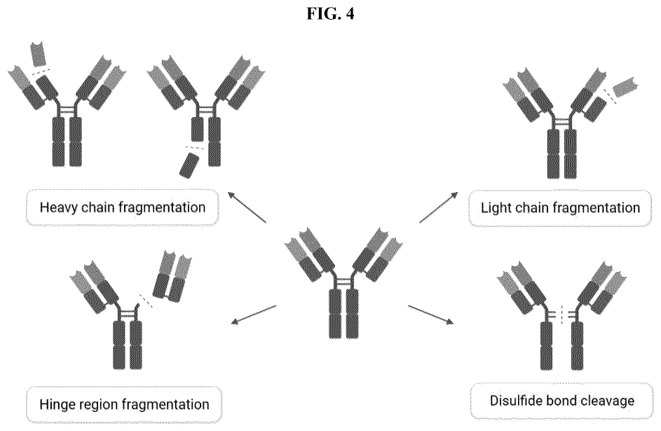

Fragmentation is one of the critical Product Quality attributes monitored to assess degradation of biopharmaceutical products, common for antibodies produced in CHO cells (Hu et al., 2021, Protein Expr. Purif., 186:105907). Fragmentation describes the breakdown of proteins into smaller fragments (LMW, low molecular weight species) due to a chemical disruption or enzymatic cleavage of covalent peptide bonds. Chemical disruption has been observed at high temperature and basic or acidic conditions (e.g. the cleavage of the hinge region in human IgG1 induced by copper at a high temperature and alkaline pH, inhibited by EDTA) (Vlasak and Ionescu, 2011, mAbs, 3:253-263). Enzymatic cleavage is caused by proteolytic enzymes (e.g. proteases or cathepsin D) (Eon-Duval et al.). Examples of immunoglobulin fragmentation sites are shown in FIG. 4 (O'Connor et al., 2017, J. Chromatogr. A, 1499:65-77).

In a biopharmaceutical manufacturing process, the proteolytic enzymes are produced by the host cells and released into the cell culture medium. Their presence leads to impurities and a decrease in stability and thus half-life of the antibody of interest (Eon-Duval et al.). The fragmentation pattern can act as a fingerprint for assessing product stability and manufacturing consistency, e.g. when a biopharmaceutical is produced in multiple sites (Torkashvand and Vaziri; Vlasak and Ionescu).

The main cleavage sites of monoclonal antibodies are located around domain interfaces and within the CH1 and CH2 (constant heavy 1 and 2) domains. The former can result in the loss of the biological activity and other functions of one or more domains, and the latter can affect the antibody's structural integrity, although the fragment could be retained by a disulfide bond. Cleavage within the variable domain could also affect the biological activity of the protein (Eon-Duval et al.).

Fragmentation occurring in the complementary determining regions (CDRs) may have an effect on the binding of the monoclonal antibody to its target. In the case of fragmentation in the hinge region, the potency may be affected by decreased function or lack thereof, depending on the presence of the Fc and Fab regions in the fragment. Fragmentation in the constant regions may affect the circulation half-time and the Fc-mediated effector function. Furthermore, fragmentation may influence aggregation rates (Vlasak and Ionescu).

Fragmentation depends on the cell culture media components. The presence of cysteine and EDTA, as well as trace elements such as zinc, manganese, and cobalt have been found to decrease fragmentation rates, while the presence of copper reportedly increases them (Torkashvand and Vaziri). Iron atoms have been found to catalyze peptide bond cleavage in the presence of histidine buffer for IgG1 molecules containing lambda light chains (Vlasak and Ionescu).

Peptide bonds are intrinsically stable under physiological conditions, but they undergo non-enzymatic hydrolysis when high temperatures or extreme pH conditions are applied. Most fragmentation occurs at cysteine, aspartate, glycine, asparagine, serine, and threonine. All of these residues but glycine have specific mechanisms of facilitating peptide bond cleavage, but other types of hydrolysis, such as mediated by water or induced by free radicals (e.g. due to prolonged exposure to H₂O₂), may occur as well (Vlasak and Ionescu).

Fragmentation has been observed to increase when the purification eluate was not neutralized from the acidic pH necessary for celution from Protein A ligands, even after short-term storage. The difference in fragmentation rates was found to be even more significant when the sample incubation temperature was increased from room temperature to 40° C. (Hu et al.).

Fragmentation can be detected and quantified based on the altered properties of proteins that underwent this process, including based on molecule size (using size-exclusion chromatography (SEC), capillary electrophoresis with sodium dodecyl sulphate (CE-SDS), or sodium dodecyl sulphate-polyacrylamide gel electrophoresis (SDS-PAGE)), and based on the chemistry of the protein's amino acid side chains (using a range of chromatography techniques such as reverse-phase HPLC, cation-exchange HPLC, or hydrophobic-interaction HPLC). Additionally, mass spectrometry or, less frequently, N-terminal sequencing can be employed to identify the cleavage site (Vlasak and Ionescu).

Size-exclusion chromatography (SEC) is often the method of choice for aggregation detection, but it can also be used to detect fragmentation in the hinge region. However, the data processing may be difficult due to poor resolution between Fc-Fab peak and the monomeric peak. Much better resolution can be achieved when using SDS-PAGE and CE-SDS, and the latter can employ a fluorescence detector, leading to improved sensitivity (Vlasak and Ionescu).

Charge variants arise when monoclonal antibodies undergo chemical degradation, including fragmentation, deamidation, isomerization, and oxidation, as well as post-translational modifications such as glycosylation, elimination of the C-terminal lysine of the heavy chain, or cyclization of N-terminal Glutamine to pyroglutamic acid (Torkashvand and Vaziri).

Among the chemical degradation pathways responsible for acidic or basic species, the two most commonly observed covalent modifications occurring in proteins and peptides are deamination and oxidation. Methionine, cysteine, histidine, tryptophan, and tyrosine are some of the amino acids that are most susceptible to oxidation: Met and Cys because of their sulfur atoms and His, Trp, and Tyr because of their aromatic rings.

Charge variants may also be caused by non-optimal cell culture conditions during the manufacturing process. The resulting heterogeneity leads to a change in the antibody's isoelectric point (pI), in vivo properties, stability, efficacy, and quality (Wu et al., 2022, TrAC Trends Anal. Chem., 150:116567), as charge heterogeneity can affect binding to other proteins and targets, thus also the tissue distribution and the pharmacokinetics of a biotherapeutic (Torkashvand and Vaziri; Zhang et al., 2023, J. Pharm. Biomed. Anal., 224:115178), especially for species with pI difference ≥1.76.

Analysis of charge variants as a cPQ attribute is therefore crucial in the manufacturing of biopharmaceutical products. In fact, monitoring charge heterogeneity is required throughout the production, storage, and transportation of biopharmaceuticals, per ICH guidelines (Wu et al.).

Charge variants with a relatively higher isoelectric point are called basic variants, whereas those with a lower pI are called acidic variants (Torkashvand and Vaziri). Basic variants have been reported to exhibit increased blood clearance and tissue retention, whereas acidic variants have been associated with decreased whole body clearance and tissue retention (Chung et al., 2018, Biotechnol. Bioeng., 115:1646-1665).

The main causes of acidic variant formation include deamidation of Asparagine residues in both the constant domains and the variable domains (especially in the complementarity-determining regions) (Torkashvand and Vaziri), and sialic acid post-translational modifications (Chung et al.).

Acidic charge variants are thought to have a greater negative impact on the pharmacokinetics and efficacy of biopharmaceuticals than basic variants, therefore their reduction is often prioritized in the manufacturing process (Chung et al.). For example, a 14-fold reduction in antigen binding has been observed to result from the deamidation of Asparagine and Glutamine (Huang et al., 2005, Anal. Chem., 77:1432-1439). Oxidation of cysteine, methionine, tryptophan, histidine, and tyrosine has been reported to reduce binding with Protein A (Bertolotti-Ciarlet, 2009, Mol. Immunol., 46:1878-1882; Gaza-Bulseco et al., 2008, J. Chromatogr. B, 870:55-62; Pan et al., 2009, Protein Sci., 18:424-433), reduce the half-life (Gaza-Bulseco et al.), and lead to loss of activity (Hensel et al., 2011, PLOS ONE, 6:e17708).

Basic species arising from the isomerization of aspartic acid (Asp-92) in a human IgG2 were found to lead to complete inactivation of the antibody (Rehder et al., 2008, Biochemistry, 47:2518-2530). However, multiple processes leading to the formation of basic species, including C-terminal modifications of lysine or arginine (Alt et al.), and N-terminal modification of glutamine and glutamic acid (Manning et al., 2010, Pharm. Res., 27:544-575), have been reported to have little to no impact on the pharmacokinetics, binding, or potency.

In certain exemplary aspects, a sample including a protein of interest can comprise more than one type of variant of a protein of interest. Such variants can include both acidic species and basic species. Acidic species are typically the variants that elute earlier than the main peak from cation exchange chromatography (CEX) or later than the main peak from anion exchange chromatography (AEX), while basic species are the variants that elute later than the main peak from CEX or earlier than the main peak from AEX. In an exemplary aspect, basic species may migrate earlier than the main peak from isoelectric focusing (IEF) and acidic species may migrate later than the main peak from IEF in normal polarity. In an exemplary aspect, basic species may migrate later than the main peak from IEF and acidic species may migrate earlier than the main peak from IEF in reverse polarity.

As used herein, the terms “acidic species,” “AS,” “acidic region,” and “AR,” refer to the variants of a protein which are characterized by an overall acidic charge.

In certain aspects, the sample can comprise more than one type of acidic species variant. For example, but not by way of limitation, the total acidic species can be categorized based on chromatographic retention time of the peaks appearing, or by UV or other absorbance peaks generated using IEF.

As used herein, the terms “oxidative species,” “OS,” or “oxidation variant” refer to the variants of a protein formed by oxidation. Such oxidative species can also be detected by various methods, such as ion exchange, for example, WCX-10 HPLC (a weak cation exchange chromatography), or IEF. Oxidation variants can result from oxidation occurring at histidine, cysteine, methionine, tryptophan, phenylalanine and/or tyrosine residues.

As used herein, the terms “basic species,” “basic region,” and “BR,” refer to the variants of a protein, for example, an antibody or antigen-binding portion thereof, which are characterized by an overall basic charge, relative to the primary charge variant species present within the protein. For example, in recombinant protein preparations, such basic species can be detected by various methods, such as ion exchange, for example, WCX-10 HPLC (a weak cation exchange chromatography), or IEF. Exemplary variants can include, but are not limited to, lysine variants, isomerization of aspartic acid, succinimide formation at asparagine, methionine oxidation, amidation, incomplete disulfide bond formation, mutation from serine to arginine, aglycosylation, fragmentation and aggregation. Commonly, basic species elute later than the main peak during CEX or earlier than the main peak during AEX analysis. (Chromatographic analysis of the acidic and basic species of recombinant monoclonal antibodies. MAbs. 2012 Sep. 1; 4 (5): 578-585. doi: 10.4161/mabs.21328, the entire teaching of which is herein incorporated by reference.)

In certain aspects, the sample can comprise more than one type of basic species variant. For example, but not by way of limitation, the total basic species can be divided based on chromatographic retention time of the peaks appearing, or based on UV or other absorbance peaks generated using IEF. Another example in which the total basic species can be divided can be based on the type of variant—for example, structure variants or fragmentation variants.

In biopharmaceutical manufacturing, it is the cell line development and the upstream process that primarily manage the control of the charge variant profile, as the downstream purification capability is limited. Cell line development includes protein engineering to control post-translational modifications, and development of the upstream process involves both optimizing the process conditions like the cell culture medium, and post-culture handling. For acidic charge variant reduction, pH and temperature are the main factors to control, whereas the formation of basic charge variants is significantly influenced by additional parameters, including dissolved oxygen and production reactor seeding density (Chung et al.).

Reducing the temperature in the bioreactor may slow down the rate of protein degradation, possibly by reducing the amount of proteolytic enzymes released from dead cells (Yoon et al., 2003, Biotechnol. Bioeng., 82:289-298). Indeed, lowering the temperature has been observed to reduce the amount of acidic charge variants, although it can increase the amount of basic charge variants formed (Torkashvand and Vaziri).

Numerous chromatographic methods have been used to characterize charge variants, including anion and cation exchange chromatography (AEX and CEX), hydrophobic interaction chromatography, liquid chromatography-mass spectrometry (LC-MS), affinity chromatography, and reverse-phase high-performance liquid chromatography (RP HPLC) (Chung et al.). However, most traditional analytical techniques of chromatography (except for ion exchange chromatography), even when coupled with mass spectrometry, cannot accurately differentiate between charge variants with very similar isoelectric points (Zhang et al., 2023), therefore other methods have been developed for this purpose, including free flow electrophoresis (FFE), isoelectric focusing gel electrophoresis (IEF), and more recently imaged capillary isoelectric focusing electrophoresis (iCIEF) (Torkashvand and Vaziri).

As used herein, “isoelectric focusing” or “IEF”, also known simply as electrofocusing, is a technique for separating charged molecules, usually proteins or peptides, on the basis of their isoelectric point (pI), for example, the pH at which the molecule has no charge. IEF works because, in an electric field, molecules in a pH gradient will migrate towards their pI. A variety of techniques for conducting IEF exist, all of which are encompassed in the term IEF as used herein where relevant. For example, in capillary isoelectric focusing (cIEF), samples travel through a capillary based on an applied electric field. A UV detector may be used at a point along the capillary to detect the time at which an analyte, such as a protein, traverses that point of the capillary. Because travel time through the capillary is directly related to the charge (pI) of the analyte, UV signal from a point in the capillary over time can be represented as a UV trace, which represents the varying charges (pI) of sample components. In an exemplary embodiment, a UV trace generated by cIEF represents charge variants of a protein of interest, with each UV peak representing a significant charge variant.

Variations of cIEF may also be used, for example, imaged cIEF (icIEF). Examples of suitable devices for performing icIEF analysis include CEInfinite (Advanced Electrophoresis Solutions), iCE3 (ProteinSimple) and Maurice (ProteinSimple). Following focusing of charge variants in a sample, icIEF analysis may further comprise a mobilization step, wherein pressure is used to mobilize a focused sample past a detection window, and may additionally mobilize a focused sample out of the separation capillary. Fractions may be collected from the mobilized sample. Collected fractions may correspond to a charge variant of interest. A fraction may comprise more than one charge variant. A charge variant may be collected in more than one fraction.

iCIEF is routinely used within the biopharmaceutical industry due to its high-throughput, high resolution, and reliability. UV detectors are employed for quantitation and to obtain a UV profile of the product, as they offer high sensitivity. Additionally, mass spectrometry, such as electrospray ionization-mass spectrometry (ESI-MS), may be applied to identify the charge variants by determining their molecular weight, although the technique is limited by interfering species such as methylcellulose or ampholytes, commonly used in many IEF methods (Zhang et al., 2023; Schlecht et al., 2022, Electrophoresis, 44 (5-6): 540-548).

iCIEF can be used to analyze a range of species, including monoclonal antibodies, antibody-drug conjugates, glycoproteins, and vaccines. Recent developments of the technique include using fluorescence or chemiluminescence for improved detection sensitivity (Wu et al.). However, iCIEF involves complicated operation and oftentimes trial-and-error optimization (Zhang et al., 2023).

Glycosylation is a post-translational protein modification regulated by glycotransferase enzymes, consisting in the attachment of oligosaccharides to a protein's polypeptide backbone, which occurs in the endoplasmic reticulum and the Golgi apparatus in the cell (Ivarsson et al., 2014, J. Biotechnol., 188:88-96; Mao et al., 2023, Biotechnol. Prog., e3365).

Glycans can be O-linked or, much more commonly, N-linked, depending on the attachment site to the protein's polypeptide backbone (serine- or threonine-linked for the former and asparagine-linked for the latter (del Val et al., 2010, Biotechnol. Prog., 26:1505-1527)). Every IgG molecule is N-glycosylated at an asparagine (Asn-297) residue in the Fc region's CH2 domain, and 15-20% of IgGs are N-glycosylated in either the light or the heavy chain of the variable domain in the Fab region. Overall, it is the Fab region in which glycosylation is more extensive due to steric hindrance of the glycotransferase enzymes in the Fc region caused by the close proximity of the heavy chains to each other (Eon-Duval et al.).

Monoclonal antibodies produced by CHO cells exhibit Fc region glycosylation patterns (glycoproteins lacking α-galactose residues (Mao et al.)) comparable to antibodies present in human blood, but CHO cells can also produce small amounts of aglycosyl and high-mannose glycoforms, which are not present among most human IgGs (Eon-Duval et al.).

Antibodies exhibit a high degree of glyco-heterogeneity: microheterogeneity—differences in glycoform distribution arising due to variation in processing within the Golgi apparatus; and macroheterogeneity—the absence or presence of a glycan on a glycosylation site caused by glycoprotein degradation or inefficiency of glycan transfer to the site (Reusch and Tejada; Mao et al.; del Val et al.).

The glycan profile of a therapeutic antibody depends on the production process parameters such as the cell culture conditions (glucose concentration (Villacrés et al., 2015, Biotechnol. J., 10:1051-1066), redox potential (Dionne et al., 2017, J. Biotechnol., 246:71-80), pH, shear stress, CO2 and ammonia content), the amount of expressed enzymes involved in the glycosylation process, and the oligosaccharide substrate accessibility (Torkashvand and Vaziri; Ivarsson et al.). The high glyco-heterogeneity of biopharmaceuticals and the significant impact that process parameter changes have on the glycan profile of antibodies make monitoring PQ attributes throughout the manufacturing process crucial to delivering drug products with consistent quality (Ivarsson et al.).

Assessing the impact of glycosylation on the efficacy and safety of a biopharmaceutical is a complex process. The glycosylation pattern can affect the pharmacokinetics, immunogenicity, and the biological activity of biotherapeutics, including increasing the solubility of proteins, decreasing the aggregation rates, preventing enzymatic degradation and thus increasing the circulatory lifetime in certain cases (Kateja et al., 2018, J. Chromatogr. A, 1579:60-72), and impacting the clearance rate and effector functions (Ivarsson et al.).

Non-glycosylated immunoglobulins exhibit loss of functions, conformational differences, increased aggregation rates, and decreased thermal stability. On the other hand, antibodies with a high degree of galactosylation have been shown to lead to increased CDC activity; high mannose glycovariants may exhibit reduced serum half-life, while a low level of core-fucose may result in increased ADCC (Reusch and Tejada).

Glycan profile is therefore one of the critical Product Quality attributes which need to be monitored in the manufacturing of therapeutic antibodies with its potential impact to efficacy and safety of the biotherapeutic in mind, as well as to reduce batch-to-batch variability and to avoid potential financial losses if a batch with undesired protein glycosylation patterns needs to be discarded.

Assessing macroheterogeneity includes identifying glycosylation sites, which can be done with collision-induced dissociation experiments and mass spectrometry (del Val et al.) or capillary electrophoresis (Mao et al.). Characterizing microheterogeneity (glycan profiling) is carried out to detect and quantify the oligosaccharides present at the glycosylation sites (del Val et al.). Techniques which have been used for the analysis of glycan profiles include capillary electrophoresis (Mao et al.), mass spectrometry (Torkashvand and Vaziri), anion exchange chromatography, fluorophore-assisted carbohydrate electrophoresis (FACE) (del Val et al.), and hydrophilic interaction liquid chromatography (HILIC) with a fluorescence detector (Villacrés et al.).

HILIC uses a polar stationary phase and a relatively polar mobile phase (for example, acetonitrile), and separates analytes based on the differences in their capacity to hydrogen-bond with the stationary phase. Glycan profiling with HILIC has been done both using conventional packed columns employed in HPLC and UPLC instruments, and with elution plates (del Val et al.; Lauber et al., 2013, Waters Application Note, 720004717EN).

### Exemplary Embodiments

This disclosure provides methods and systems for enriching a protein of interest from a sample, for example a cell culture sample. In some exemplary embodiments, the methods can comprise (a) contacting a cell culture sample including a protein of interest to a centrifugal (spin) column including an affinity resin to produce an immobilized sample, wherein the affinity resin specifically binds to the protein of interest; (b) subjecting the immobilized sample to at least one washing step; and (c) subjecting the immobilized sample from (b) to at least one elution step to produce an enriched protein of interest.

In one aspect, the protein of interest is selected from a group consisting of a therapeutic protein, a receptor, an antigen-binding protein, an antibody, a monoclonal antibody, a multispecific antibody, a bispecific antibody, an antibody-derived protein, a fusion protein, a receptor fusion protein, a trap protein, a fragment thereof, a variant thereof, and a combination thereof. In another aspect, the protein of interest is a monoclonal antibody. In a further aspect, the protein of interest is dupilumab (DUPIXENT®).

In one aspect, the protein of interest comprises an Fc domain.

In one aspect, the protein of interest is selected from a group consisting of an Fc-fusion protein, a receptor-Fc-fusion protein, and a ScFv-Fc fusion protein. In another aspect, the protein of interest is an anti-vascular endothelial growth factor (VEGF) antibody or an anti-VEGF receptor-Fc-fusion protein. In a particular aspect, the protein of interest is aflibercept.

In one aspect, the protein of interest is a recombinant protein.

In one aspect, the cell culture sample is from a cell culture selected from a eukaryotic cell culture, a mammalian cell culture, or an insect cell culture. In another aspect, the cell culture sample is selected from a group consisting of CHO, CHO-K1, CHO DUX B-11, Veggie-CHO, GS-CHO, S-CHO, CHO lec, COS, Vero, CV-1, HEK293, MCDK, HaK, BHK2, HeLa, HepG2, WI38, MRC 5, Colo25, HB 8065, HL-60, Jurkat, Daudi, A431, U937, 3T3, L cell, C127, SP2/0, NS-0, MMT, variations thereof, and combinations thereof.

In one aspect, the cell culture sample is from a CHO cell culture, a CHO-K1 cell culture, a BHK cell culture, a HEK293 cell culture, a Sf9 cell culture, or a variation thereof.

In one aspect, the cell culture sample is a clarified cell culture sample.

In one aspect, the method further comprises clarifying a cell culture sample prior to step (a). In a specific aspect, the clarifying comprises centrifugation, filtration, and/or precipitation of insoluble components.

In one aspect, the cell culture sample is an upstream cell culture sample.

In one aspect, the cell culture sample is taken from a cell culture at a day from day 1 to day 20, day 3 to day 15, day 3 to day 12, day 3 to day 10, day 5 to day 10, day 5 to day 12, or day 5 to day 13. In another aspect, the cell culture sample is taken from a cell culture at a day selected from a group consisting of day 1, 2, 3, 4, 5, 6, 7, 8, 9, 10, 11, 12, 13, 14, 15, 16, 17, 18, 19, or 20.

In one aspect, the method further comprises repeating steps (a)-(c) (contacting, washing, and eluting) at least once. In a specific aspect, the method is repeated using at least a first cell culture sample and a second cell culture sample taken from the same cell culture. In a more specific aspect, the first cell culture sample is taken at a first day and the second cell culture sample is taken at a second day. In another specific aspect, the first cell culture sample and the second cell culture sample are taken with a time between samples of about 3 hours, about 6 hours, about 12 hours, about 18 hours, about 24 hours, about 1 day, about 2 days, about 3 days, about 4 days, about 5 days, about 6 days, about 7 days, about 8 days, about 9 days, or about 10 days.

In one aspect, the method further comprises performing steps (a)-(c) (contacting, washing, and eluting) with at least two cell culture samples in parallel. In a specific aspect, the at least two cell culture samples are from two different cell cultures.

In one aspect, the contacting step comprises combining the cell culture sample and a binding buffer. In a specific aspect, the binding buffer comprises Tris-buffered saline or sodium phosphate. In a further specific aspect, the binding buffer further comprises sodium chloride. In a further specific aspect, a concentration of sodium phosphate is from 10 mM to 30 mM, from 15 mM to 25 mM, about 10 mM, about 15 mM, about 20 mM, about 25 mM, or about 30 mM. In a further specific aspect, a concentration of sodium chloride is from 100 mM to 500 mM, about 100 mM, about 200 mM, about 300 mM, about 400 mM, or about 500 mM. In an additional specific aspect, a pH of the binding buffer is from 7.1 to 7.3.

In one aspect, the binding buffer comprises Tris-buffered saline, sodium phosphate, HEPES, or Tris. In an additional aspect, the binding buffer further comprises sodium chloride or calcium chloride. In an additional specific aspect, a pH of the binding buffer is from 6.0 to 8.0

In one aspect, the contacting step comprises adding to the column a combined volume of a binding buffer and the cell culture sample of from 250 to 1000 μL, from 300 to 900 μL, from 400 to 800 μL, from 500 to 700 μL from 550 to 650 μL, from 590 to 610 μL, from 599 to 601 μL, or about 600 μL.

In one aspect, the contacting step comprises adding to the column a volume of the cell culture sample of from 50 to 100 μL, from 60 to 90 μL, from 70 to 80 μL, about 70 μL, about 71 μL, about 72 μL, about 73 μL, about 74 μL, about 75 μL, about 76 μL, about 77 μL, about 78 μL, about 79 μL, or about 80 μL.

In one aspect, the contacting step comprises adding to the column an amount of protein of from 100.5 μg to 804 μg, 250 μg to 1 g, from 350 μg to 900 μg, from 450 μg to 804 μg, from 500 μg to 700 μg, from 550 μg to 650 μg, from 575 μg to 625 μg, from 590 μg to 610 μg, about 595 μg, about 596 μg, about 597 μg, about 598 μg, about 599 μg, about 600 μg, about 601 μg, about 602 μg, about 603 μg, about 604 μg, or about 605 μg.

In one aspect, the affinity resin is Protein A resin, Protein G resin, or a combination thereof.

In one aspect, the at least one washing step comprises adding a washing buffer to the column and centrifuging the column to produce a washed flowthrough. In a specific aspect, the washing buffer comprises Tris-buffered saline, sodium acetate, or sodium phosphate. In a more specific aspect, the washing buffer further comprises sodium chloride. In a further specific aspect, a concentration of sodium phosphate or sodium acetate is from 10 mM to 30 mM, from 15 mM to 25 mM, about 10 mM, about 15 mM, about 20 mM, about 25 mM, or about 30 mM. In a further specific aspect, a concentration of sodium chloride is from 100 mM to 500 mM, about 100 mM, about 200 mM, about 300 mM, about 400 mM, or about 500 mM. In an additional specific aspect, a pH of the washing buffer is from 7.1 to 7.3.

In one aspect, the washing buffer comprises Tris-buffered saline, sodium phosphate, or sodium acetate, HEPES, or Tris. In a specific aspect, the washing buffer further comprises sodium chloride or calcium chloride. In an additional specific aspect, a pH of the washing buffer is from 6.0 to 8.0.

In another specific aspect, a volume of the washing buffer is about 600 μL. In yet another specific aspect, the centrifuging is performed at about 100 relative centrifugal force (RCF). In still another specific aspect, the centrifuging is performed for about 30 seconds, about 1 minute, about 90 seconds, or about 2 minutes.

In one aspect, a number of washing steps is one, two, or three. In another aspect, the binding buffer and the washing buffer are the same.

In one aspect, the at least one elution step comprises adding an elution buffer to the column and centrifuging the column to produce an eluate. In a specific aspect, the elution buffer comprises acetic acid or glycine. In a more specific aspect, a concentration of the acetic acid is from 0.1% to 0.3%, from 0.12% to 0.27%, from 0.12% to 0.24%, about 0.12%, about 0.24%, from 15 mM to 50 mM, from 16 mM to 45 mM, from 20 mM to 40 mM, about 15 mM, about 16 mM, about 20 mM, about 25 mM, about 30 mM, about 35 mM, about 40 mM, about 45 mM, or about 50 mM. In another specific aspect, a concentration of the glycine is about 0.1 M. In yet another specific aspect, a volume of the elution buffer is from 300 μL to 500 μL, from 350 μL to 450 μL, about 300 μL, about 350 μL, about 400 μL, about 450 μL, or about 500 μL. In still another specific aspect, the centrifuging is performed at about 100 RCF. In a further specific aspect, the centrifuging is performed for about 30 seconds, about 1 minute, about 90 seconds, or about 2 minutes.

In one aspect, a pH of the elution buffer is below 4, from 1 to 4, from 2 to 4, from 2.5 to 3.5, from 2.8 to 3.2, about 1, about 1.5, about 2, about 2.5, about 3, or about 3.5.

In one aspect, a number of elution steps is one, two, or three.

In one aspect, the at least one elution step comprises adding a neutralizing buffer to the column. In a specific aspect, the neutralizing buffer comprises Tris base. In a more specific aspect, a concentration of the Tris base is from 1 M to 2 M, about 1 M, about 1.5 M, or about 2 M. In another specific aspect, a volume of the neutralizing buffer is from 5 μL to 50 μL, about 5 μL, about 10 μL, about 20 μL, or about 30 μL.

In one aspect, a yield of the enriched protein of interest is above 50%, above 60%, above 70%, above 80%, above 90%, above 95%, above 99%, about 60%, about 70%, about 75%, about 80%, about 85%, about 90%, about 91%, about 92%, about 93%, about 94%, about 95%, about 96%, about 97%, about 98%, about 99%, or about 100%.

In one aspect, an amount of protein in the enriched protein of interest is above 10 μg, above 20 μg, above 50 μg, above 100 μg, above 200 μg, above 300 μg, above 400 μg, above 500 μg, above 600 μg, above 700 μg, above 800 μg, above 900 μg, above 1000 μg, above 1100 μg, above 1200 μg, about 10 μg, about 20 μg, about 50 μg, about 100 μg, about 200 μg, about 300 μg, about 400 μg, about 500 μg, about 600 μg, about 700 μg, about 800 μg, about 900 μg, about 1000 μg, about 1100 μg, or about 1200 μg.

In one aspect, a concentration of the enriched protein of interest is above 0.01 μg/μL, above 0.05 μg/μL, above 0.1 μg/μL, above 0.2 μg/μL, above 0.5 μg/μL, above 1 μg/μL, above 2 μg/μL, about 0.05 μg/μL, about 0.1 μg/μL, about 0.2 μg/μL, about 0.5 μg/μL, about 1 μg/μL, about 1.5 μg/μL, about 2 μg/μL, or about 2.5 μg/μL.

In one aspect, the column may be reused for performing the method at least twice.

In one aspect, a duration of the method is less than 24 hours, less than 12 hours, less than 6 hours, less than 3 hours, less than 2 hours, less than 1 hour, less than 30 minutes, about 3 hours, about 2 hours, about 1.5 hours, about 1 hour, about 50 minutes, about 45 minutes, about 40 minutes, about 30 minutes, or about 20 minutes.

In one aspect, the method further comprises characterizing the enriched protein of interest. In another aspect, the method further comprises characterizing at least one product quality attribute of the enriched protein of interest. In a specific aspect, the at least one product quality attribute is a critical product quality attribute. In yet another aspect, the method further comprises subjecting the enriched protein of interest to chromatography, mass spectrometry, spectroscopy, capillary electrophoresis, gel electrophoresis, and/or a ligand binding assay.

In one aspect, the method further comprises characterizing at least one size variant of the enriched protein of interest. In another aspect, the method further comprises characterizing at least one high molecular weight species of the enriched protein of interest. In a specific aspect, the characterizing comprises subjecting the enriched protein of interest to size exclusion chromatography (SEC) analysis.

In one aspect, the method further comprises characterizing at least one fragment of the enriched protein of interest. In a specific aspect, the characterizing comprises subjecting the enriched protein of interest to capillary electrophoresis with sodium dodecyl sulfate (CE-SDS) analysis.

In one aspect, the method further comprises characterizing at least one charge variant of the enriched protein of interest. In a specific aspect, the characterizing comprises subjecting the enriched protein of interest to imaged capillary isoelectric focusing electrophoresis (iCIEF).

In one aspect, the method further comprises characterizing at least one glycan of the enriched protein of interest. In a specific aspect, the characterizing comprises subjecting the enriched protein of interest to hydrophilic interaction chromatography (HILIC) analysis.

In a specific aspect, the method further comprises using the at least one product quality attribute to determine whether the cell culture should be continued or discontinued. In another specific aspect, the method further comprises using the at least one product quality attribute to determine whether the cell culture should be modified. In a further specific aspect, modifications to a cell culture may include modifications to the temperature, pH, dissolved oxygen or other gasses, or cell culture medium.

This disclosure also provides methods for monitoring at least one product quality attribute of a recombinant protein of interest. In some exemplary embodiments, the methods may comprise (a) obtaining a first cell culture sample including a recombinant protein of interest from a first time point; (b) contacting the first cell culture sample to a centrifugal column including an affinity resin to produce an immobilized sample, wherein the affinity resin specifically binds to the protein of interest; (c) subjecting the immobilized sample to at least one washing step; (d) subjecting the immobilized sample from (c) to at least one elution step to produce a first enriched sample; (e) repeating steps (b)-(d) with at least one additional cell culture sample including a recombinant protein of interest from at least one additional time point to produce at least one additional enriched sample, wherein the first cell culture sample and the at least one additional cell culture sample are obtained from the same cell culture; and (f) characterizing at least one product quality attribute from the first enriched sample and the at least one additional enriched sample to monitor at least one product quality attribute of the recombinant protein of interest.

In one aspect, the recombinant protein of interest is selected from a group consisting of a therapeutic protein, a receptor, an antigen-binding protein, an antibody, a monoclonal antibody, a multispecific antibody, a bispecific antibody, an antibody-derived protein, a fusion protein, a receptor fusion protein, a trap protein, a fragment thereof, a variant thereof, and a combination thereof. In another aspect, the recombinant protein of interest is a monoclonal antibody. In a further aspect, the recombinant protein of interest is dupilumab (DUPIXENT®).

In one aspect, the recombinant protein of interest comprises an Fc domain.

In one aspect, the recombinant protein of interest is selected from a group consisting of an Fc-fusion protein, a receptor-Fc-fusion protein, and a ScFv-Fc fusion protein. In another aspect, the recombinant protein of interest is an anti-vascular endothelial growth factor (VEGF) antibody or an anti-VEGF receptor-Fc-fusion protein. In a particular aspect, the recombinant protein of interest is aflibercept.

In one aspect, the cell culture is a eukaryotic cell culture, a mammalian cell culture, or an insect cell culture. In another aspect, the cell culture is selected from a group consisting of CHO, CHO-K1, CHO DUX B-11, Veggie-CHO, GS-CHO, S-CHO, CHO lec, COS, Vero, CV-1, HEK293, MCDK, HaK, BHK2, HeLa, HepG2, WI38, MRC 5, Colo25, HB 8065, HL-60, Jurkat, Daudi, A431, U937, 3T3, L cell, C127, SP2/0, NS-0, MMT, variations thereof, and combinations thereof.

In one aspect, the cell culture is a CHO cell culture, a CHO-K1 cell culture, a BHK cell culture, a HEK293 cell culture, a Sf9 insect cell culture, or a variation thereof.

In one aspect, the first cell culture sample and the at least one additional cell culture sample are clarified cell culture samples.

In one aspect, the method further comprises clarifying the first cell culture sample and the at least one additional cell culture sample prior to step (a). In a specific aspect, the clarifying comprises centrifugation, filtration, and/or precipitation of insoluble components.

In one aspect, the first cell culture sample and the at least one additional cell culture sample are upstream cell culture samples.

In one aspect, the first cell culture sample and/or the at least one additional cell culture sample are taken from a cell culture at a day from day 1 to day 20, day 3 to day 15, day 3 to day 12, day 3 to day 10, day 5 to day 10, day 5 to day 12, or day 5 to day 13. In another aspect, the first cell culture sample and/or the at least one additional cell culture sample are taken from a cell culture at a day selected from a group consisting of day 1, 2, 3, 4, 5, 6, 7, 8, 9, 10, 11, 12, 13, 14, 15, 16, 17, 18, 19, or 20.

In one aspect, the first cell culture sample and the at least one additional cell culture sample are taken with a time between samples of about 3 hours, about 6 hours, about 12 hours, about 18 hours, about 24 hours, about 1 day, about 2 days, about 3 days, about 4 days, about 5 days, about 6 days, about 7 days, about 8 days, about 9 days, or about 10 days.

In one aspect, steps (b)-(d) of the method are performed with the first cell culture sample and the at least one additional cell culture sample in parallel.

In one aspect, the contacting step comprises combining the first cell culture sample and/or the at least one additional cell culture sample and a binding buffer. In a specific aspect, the binding buffer comprises Tris-buffered saline or sodium phosphate. In a further specific aspect, a concentration of sodium phosphate is from 10 mM to 30 mM, from 15 mM to 25 mM, about 10 mM, about 15 mM, about 20 mM, about 25 mM, or about 30 mM. In an additional specific aspect, a pH of the binding buffer is from 7.1 to 7.3.

In one aspect, the contacting step comprises adding to the column a combined volume of a binding buffer and a cell culture sample of from 250 to 1000 μL, from 300 to 900 μL, from 400 to 800 μL, from 500 to 700 μL from 550 to 650 μL, from 590 to 610 μL, from 599 to 601 μL, or about 600 μL.

In one aspect, the contacting step comprises adding to the column a volume of the first cell culture sample or the at least one additional cell culture sample of from 50 to 100 μL, from 60 to 90 μL, from 70 to 80 μL, about 70 μL, about 71 μL, about 72 μL, about 73 μL, about 74 μL, about 75 μL, about 76 μL, about 77 μL, about 78 μL, about 79 μL, or about 80 μL.

In one aspect, the contacting step comprises adding to the column an amount of protein of from 100.5 μg to 804 μg, 250 μg to 1 g, from 350 μg to 900 μg, from 450 μg to 804 μg, from 500 μg to 700 μg, from 550 μg to 650 μg, from 575 μg to 625 μg, from 590 μg to 610 μg, about 595 μg, about 596 μg, about 597 μg, about 598 μg, about 599 μg, about 600 μg, about 601 μg, about 602 μg, about 603 μg, about 604 μg, or about 605 μg.

In one aspect, the affinity resin is Protein A resin, Protein G resin, or a combination thereof.

In one aspect, the at least one washing step comprises adding a washing buffer to the column and centrifuging the column to produce a washed flowthrough. In a specific aspect, the washing buffer comprises Tris-buffered saline, sodium acetate, or sodium phosphate, or sodium acetate. In a further specific aspect, a concentration of sodium phosphate or sodium acetate is from 10 mM to 30 mM, from 15 mM to 25 mM, about 10 mM, about 15 mM, about 20 mM, about 25 mM, or about 30 mM. In an additional specific aspect, a pH of the washing buffer is from 7.1 to 7.3. In another specific aspect, a volume of the washing buffer is about 600 μL. In yet another specific aspect, the centrifuging is performed at about 100 relative centrifugal force (RCF). In still another specific aspect, the centrifuging is performed for about 30 seconds, about 1 minute, about 90 seconds, or about 2 minutes.

In one aspect, a number of washing steps is one, two, or three. In another aspect, the binding buffer and the washing buffer are the same.

In one aspect, the at least one elution step comprises adding an elution buffer to the column and centrifuging the column to produce an eluate. In a specific aspect, the elution buffer comprises acetic acid or glycine. In a more specific aspect, a concentration of the acetic acid is from 0.1% to 0.3%, from 0.12% to 0.27%, from 0.12% to 0.24%, about 0.12%, about 0.24%, from 15 mM to 50 mM, from 16 mM to 45 mM, from 20 mM to 40 mM, about 15 mM, about 16 mM, about 20 mM, about 25 mM, about 30 mM, about 35 mM, about 40 mM, about 45 mM, or about 50 mM. In another specific aspect, a concentration of the glycine is about 0.1 M. In yet another specific aspect, a volume of the elution buffer is from 300 μL to 500 μL, from 350 μL to 450 μL, about 300 μL, about 350 μL, about 400 μL, about 450 μL, or about 500 μL. In still another specific aspect, the centrifuging is performed at about 100 RCF. In a further specific aspect, the centrifuging is performed for about 30 seconds, about 1 minute, about 90 seconds, or about 2 minutes.

In one aspect, a pH of the elution buffer is below 4, from 1 to 4, from 2 to 4, from 2.5 to 3.5, from 2.8 to 3.2, about 1, about 1.5, about 2, about 2.5, about 3, or about 3.5.

In one aspect, a number of elution steps is one, two, or three.

In one aspect, the at least one elution step comprises adding a neutralizing buffer to the column. In a specific aspect, the neutralizing buffer comprises Tris base. In a more specific aspect, a concentration of the Tris base is from 1 M to 2 M, about 1 M, about 1.5 M, or about 2 M. In another specific aspect, a volume of the neutralizing buffer is from 5 μL to 50 μL, about 5 μL, about 10 μL, about 20 μL, or about 30 μL.

In one aspect, a yield of the enriched protein of interest is above 50%, above 60%, above 70%, above 80%, above 90%, above 95%, above 99%, about 60%, about 70%, about 75%, about 80%, about 85%, about 90%, about 91%, about 92%, about 93%, about 94%, about 95%, about 96%, about 97%, about 98%, about 99%, or about 100%.

In one aspect, an amount of protein in the enriched protein of interest is above 10 μg, above 20 μg, above 50 μg, above 100 μg, above 200 μg, above 300 μg, above 400 μg, above 500 μg, above 600 μg, above 700 μg, above 800 μg, above 900 μg, above 1000 μg, above 1100 μg, above 1200 μg, about 10 μg, about 20 μg, about 50 μg, about 100 μg, about 200 μg, about 300 μg, about 400 μg, about 500 μg, about 600 μg, about 700 μg, about 800 μg, about 900 μg, about 1000 μg, about 1100 μg, or about 1200 μg.

In one aspect, a concentration of the enriched protein of interest is above 0.01 μg/μL, above 0.05 μg/μL, above 0.1 μg/μL, above 0.2 μg/μL, above 0.5 μg/μL, above 1 μg/μL, above 2 μg/μL, about 0.05 μg/μL, about 0.1 μg/μL, about 0.2 μg/μL, about 0.5 μg/μL, about 1 μg/μL, about 1.5 μg/μL, about 2 μg/μL, or about 2.5 μg/μL.

In one aspect, the column may be reused for performing the method at least twice.

In one aspect, a duration of the method is less than 24 hours, less than 12 hours, less than 6 hours, less than 3 hours, less than 2 hours, less than 1 hour, less than 30 minutes, about 3 hours, about 2 hours, about 1.5 hours, about 1 hour, about 50 minutes, about 45 minutes, about 40 minutes, about 30 minutes, or about 20 minutes.

In one aspect, the characterizing comprises subjecting the first enriched sample and the at least one additional enriched sample to chromatography, mass spectrometry, spectroscopy, capillary electrophoresis, gel electrophoresis, and/or a ligand binding assay.

In one aspect, the at least one product quality attribute comprises at least one size variant, high molecular weight species, or low molecular weight species of the recombinant protein of interest. In a specific aspect, the characterizing comprises subjecting the first enriched sample and the at least one additional enriched sample to size exclusion chromatography (SEC) analysis.

In one aspect, the at least one product quality attribute comprises at least one fragment of the recombinant protein of interest. In a specific aspect, the characterizing comprises subjecting the first enriched sample and the at least one additional enriched sample to capillary electrophoresis with sodium dodecyl sulfate (CE-SDS) analysis.

In one aspect, the at least one product quality attribute comprises at least one charge variant of the recombinant protein of interest. In a specific aspect, the characterizing comprises subjecting the first enriched sample and the at least one additional enriched sample to imaged capillary isoelectric focusing electrophoresis (iCIEF).

In one aspect, the at least one product quality attribute comprises at least one glycosylation variant of the recombinant protein of interest. In a specific aspect, the characterizing comprises subjecting the first enriched sample and the at least one additional enriched sample to hydrophilic interaction chromatography (HILIC) analysis.

In a specific aspect, the method further comprises using the at least one product quality attribute to determine whether the cell culture should be continued or discontinued. In another specific aspect, the method further comprises using the at least one product quality attribute to determine whether the cell culture should be modified. In a further specific aspect, modifications to a cell culture may include modifications to the temperature, pH, dissolved oxygen or other gasses, or cell culture medium.

This disclosure additionally provides methods for profiling product quality attributes of an antibody of interest from an upstream cell culture sample. In some exemplary embodiments, the methods can comprise (a) contacting an upstream cell culture sample including an antibody of interest to a centrifugal column including a Protein A resin to produce an immobilized sample; (b) subjecting the immobilized sample to at least one washing step; (c) subjecting the immobilized sample from (b) to at least one elution step to produce an enriched antibody of interest; and (d) characterizing at least two product quality attributes of the enriched antibody of interest to profile product quality attributes of the antibody of interest, wherein the at least two product quality attributes comprise aggregation, charge variation, glycosylation, and/or fragmentation.

This disclosure provides further methods for assessing product quality attributes of an antibody of interest from an upstream cell culture sample. In some exemplary embodiments, the methods can comprise (a) contacting a clarified upstream cell culture sample including an antibody of interest and a binding buffer comprising about 20 mM sodium phosphate (pH 7.10-7.30) to a centrifugal column including a Protein A resin to produce an immobilized sample, wherein a protein content of the clarified upstream cell culture sample is about 600 μg and a combined volume of the clarified upstream cell culture sample and the binding buffer is about 600 μL; (b) subjecting the immobilized sample to two washing steps, wherein each washing step includes adding about 600 μL of about 20 mM sodium phosphate (pH 7.10-7.30) to the column and centrifuging the column at about 100 RCF for about 1 minute; (c) subjecting the immobilized sample from (b) to two elution steps to produce one eluate, wherein the elution step includes adding about 400 μL of about 40 mM acetic acid (pH 2.80-3.20) to the column, agitating the column, adding about 30 μL of about 2 M Tris base to the column, and centrifuging the column at about 100 RCF for about 1 minute; and (d) characterizing at least two product quality attributes of the enriched antibody of interest to profile product quality attributes of the antibody of interest, wherein the at least two product quality attributes comprise aggregation, charge variation, glycosylation, and/or fragmentation. In one aspect, the protein of interest is dupilumab.

In some exemplary embodiments, the methods can comprise (a) contacting a clarified upstream cell culture sample including an antibody of interest and a binding buffer comprising about 10 mM sodium phosphate and 500 mM NaCl to a centrifugal column including a Protein A resin to produce an immobilized sample, wherein a protein content of the clarified upstream cell culture sample is about 600 μg and a combined volume of the clarified upstream cell culture sample and the binding buffer is about 600 μL; (b) subjecting the immobilized sample to two washing steps, wherein the first washing step includes adding about 600 L of about 10 mM sodium phosphate and 500 mM NaCl to the column, the second washing step includes adding about 600 μL of about 20 mM sodium acetate, and centrifuging the column at about 100 RCF for about 1 minute; (c) subjecting the immobilized sample from (b) to two elution steps to produce one eluate, wherein the elution step includes adding about 400 μL of about 20 mM acetic acid to the column, agitating the column, adding about 5 μL of about 2 M Tris base to the column, and centrifuging the column at about 100 RCF for about 1 minute; and (d) characterizing at least two product quality attributes of the enriched antibody of interest to profile product quality attributes of the antibody of interest, wherein the at least two product quality attributes comprise aggregation, charge variation, glycosylation, and/or fragmentation. In one aspect, the protein of interest is aflibercept.

It is understood that the present invention is not limited to any of the afore mentioned protein(s), protein(s) of interest, polypeptide(s) of interest, recombinant protein(s), antibody(s), antibody fragment(s), cell(s), cell type(s), cell line(s), cell culture media, protein alkylating agent(s), protein denaturing agent(s), protein reducing agent(s), digestive enzyme(s), sample(s), chromatographic method(s), or product quality attribute(s), and any protein(s), protein(s) of interest, polypeptide(s) of interest, recombinant protein(s), antibody(s), antibody fragment(s), cell(s), cell type(s), cell line(s), cell culture media, protein alkylating agent(s), protein denaturing agent(s), protein reducing agent(s), digestive enzyme(s), sample(s), chromatographic method(s), or product quality attribute(s) can be selected by any suitable means.

The present invention will be more fully understood by reference to the following examples. They should not, however, be construed as limiting the scope of the invention.

### EXAMPLES

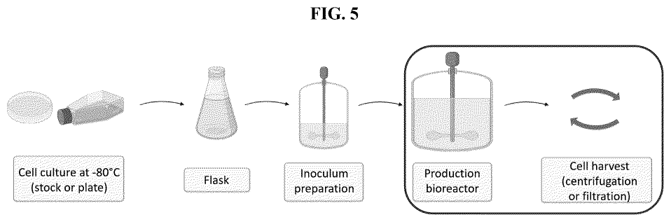

Cell culture medium. Cell culture medium containing genetically modified Chinese hamster ovary (CHO) cells, the antibody of interest, other proteins, and media components was sourced from an in-house production floor at varying stages of the upstream manufacturing process in the production bioreactor and at cell harvest, as illustrated in FIG. 5. Dupilumab was produced in Chinese hamster ovary (CHO) cells and cultured and harvested using chemically defined media (CDM) in a laboratory scale or mini bioreactor. Aflibercept was produced in CHO cells and cultured and harvested using soy hydrolysate in a mini bioreactor. Harvest samples were stored at 2-8° C. or −80° C. until use. Daily manufacturing samples obtained from the bioreactors at one day intervals or at day 10.4 prior to harvesting the entire cell culture batch. Samples were clarified by centrifugation to remove the CHO cells, and the supernatant was collected, aliquoted, and stored −80° C. until use.

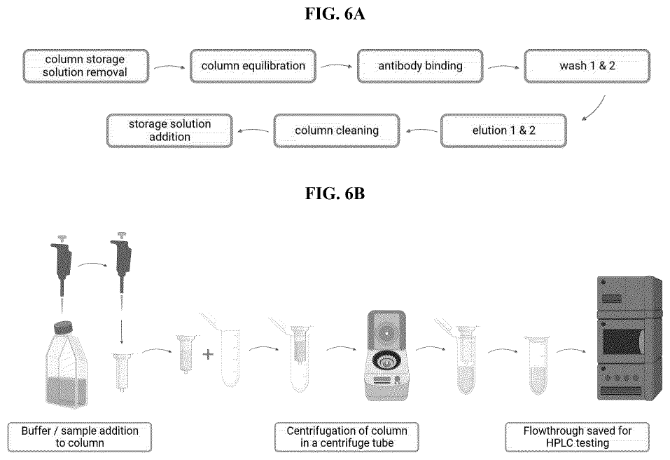

Method development for antibody purification. Antibody purification was carried out using Protein A affinity chromatography, employing commercially available spin columns with Protein A (ProA) resin. Initially, the column manufacturer's user guide and the buffer kits produced by the same manufacturer was relied upon for the purification method with minor adjustments. The subsequent runs were then carried out using optimized buffers produced in-house (see Tables 1 and 2) and the manufacturer's protocol with minor variations and additional steps (see Tables 1 and 2). The flowthroughs and eluates obtained, along with the filtered cell culture samples, were analyzed using high performance liquid chromatography (HPLC), with a photodiode array (PDA) detector or a UV detector. The concentrations of the first eluates for each sample load were analyzed by UV-visible spectrometry using Solo-VPE. Each titer assay run included an internal standard specific for the antibody of interest, used for calibration. A workflow of the overall Protein A chromatography method is illustrated in FIG. 6A. A general workflow of each step of the Protein A chromatography method is illustrated in FIG. 6B.

The protein purification protocol provided by the ProA column manufacturer and the initial adjustments made are described below. Six replicates were used for this experiment, and the obtained samples-flowthroughs after antibody binding, washing and elution steps, and the cell culture sample—were analyzed using HPLC.

Buffer preparation. Manufacturer's buffers were prepared by following the instructions attached to the dedicated buffer kit. The binding/washing buffer was prepared by diluting 5 mL of Tris-buffered saline (TBS buffer, containing 0.5 M Tris and 1.5 M NaCl) ten times with 45 mL of water. The recommended elution buffer (2.5% acetic acid) did not require dilution, according to the manual. The recommended neutralizing buffer (1 M Tris-HCl) was not present in the buffer kit. It was prepared by diluting 2 M Tris Base with water in a 1:1 ratio.

In-house buffers used for the large-scale downstream manufacturing process were tested and compared to the manufacturer's buffers (see Tables 1 and 2). A summary of the original and improved protein purification methods for dupilumab is shown in Table 1. A summary of the original and improved purification methods for aflibercept is shown in Table 2.

TABLE 1
Summary of buffers, sample volumes, centrifuge settings used
throughout the dupilumab purification optimization process,

| Protocol | Binding/washing | | Neutralizing | Cell culture | Centrifuge |
|---|---|---|---|---|---|
| version | buffer | Elution buffer | buffer | sample volume | settings |
| Manufacturer's | TBS (600 μL) | 2.5% acetic | 1M Tris Base | Max 600 μL | 70-100 |
| | | acid (400 μL) | (30 μL) | | RCF, 30 |
| | | | | | seconds |
| Version 1 | TBS (600 μL) | 2.5% acetic | 1M Tris Base | 400 μL (~3160 μg of | 100 RCF, |
| | | acid (400 μL) | (30 μL) | protein) | 1 minute |
| Version 2 | TBS (600 μL) | 2.5% acetic | 1M Tris Base | 76 μL (~600 μg of | 100 RCF, |
| | | acid (400 μL) | (30 μL) | protein) + 524 μL | 1 minute |
| | | | | binding/washing buffer | |
| Version 3 | TBS (600 μL) | 0.1M glycine | 2M Tris Base | 76 μL (~600-620 μg of | 100 RCF, |
| | | (400 μL) | (5 μL) | protein) + 524 μL | 1 minute |
| | | | | binding/washing buffer | |
| Version 4 | 20 mM sodium | 0.24% acetic | 2M Tris Base | 76 μL (~600-620 μg of | 100 RCF, |
| | phosphate (600 | acid (400 μL) | (10 μL) | protein) + 524 μL | 1 minute |
| | μL) | | | binding/washing buffer | |
| Version 5 | 20 mM sodium | 0.24% acetic | 2M Tris Base | 76 μL (~600-620 μg of | 100 RCF, |
| | phosphate (600 | acid (400 μL) | (5 μL) | protein) + 524 μL | 1 minute |
| | μL) | | | binding/washing buffer | |

TABLE 2
Summary of buffers, sample volumes, centrifuge settings
used for aflibercept purification optimization process,

| Protocol | Binding/washing | | Neutralizing | Cell culture | Centrifuge |
|---|---|---|---|---|---|
| version | buffer | Elution buffer | buffer | sample volume | settings |
| Manufacturer's | TBS (600 μL) | 2.5% acetic | 1M Tris Base | Max 600 μL | 70-100 |
| | | acid (400 μL) | (30 μL) | | RCF, 30 |
| | | | | | seconds |
| Version 1 | 10 mM sodium | 0.12% acetic | 2M Tris Base | ~600-620 μg of | 100 RCF, |
| | phosphate, 500 | acid (400 μL) | (30 μL) | protein + 10 mM sodium | 1 minute |
| | mM NaCl (600 | | | phosphate, 500 | |
| | μL) | | | mM NaCl (600 μL) | |
| | Second wash | | | | |
| | buffer: 20 mM | | | | |
| | sodium acetate | | | | |
| | (600 μL) | | | | |

Storage solution removal. The sediment in the ProA columns was first resuspended by inversion. The bottom cap was then opened, and the columns were placed in 2 mL centrifuge tubes. The column's manual suggested centrifugation at 70-100 RCF (relative centrifugal force) for 30 seconds for each step; however, the time was adjusted to 1 minute, as this was the shortest centrifugation time possible on the available instruments. To account for the longer centrifugation, the lower end of the RCF range (70 RCF) was selected. This resulted in an incomplete transfer of the solution from the columns to the centrifuge tubes. The columns were centrifuged again at 100 RCF for 1 minute, and from then on, each centrifugation step was carried out using these settings. The flowthrough was discarded, and the column was placed in a new centrifuge tube.

Equilibration. 600 μL of the binding/washing buffer was added to each column, and the medium was resuspended by inversion. The columns were centrifuged, the flowthrough was discarded, and the columns were placed in new tubes.

Antibody binding. The manufacturer's manual stated that a maximum of 600 μL of the antibody sample can be added to the column. The initial protocol was optimized to avoid column overloading by reducing the volume of cell culture sample added to the column from 400 μL to 76 μL with 524 μL of the binding/washing buffer, targeting a column load of approximately 600 μg. Subsequently, the protocol was modified by determining a sample volume based on the theoretical volume load calculated from the linearity and time course study, and adding binding buffer to the sample to reach a total volume of 600 μL (Table 3). The columns were agitated approximately every 20 seconds while incubating for 4 minutes, centrifuged, and placed in new tubes. The flowthroughs and eluate were saved for testing.

TABLE 3
Example of theoretical column load, and volume of antibody and
binding buffer determined based on theoretical column load.

| | |
|---|---|
| Theoretical column load (μg) | 603 |
| Volume of antibody solution (μL) | 450 |
| Volume of binding buffer (μL) | 150 |

Wash 1 and 2. The columns were washed twice by the addition of binding/washing buffer to the column, followed by centrifugation, and transferring the column to a new centrifuge tube. For the purification of aflibercept, the columns were washed a second time by the addition of 600 μL of 20 mM sodium acetate (see Table 2). The flowthroughs were saved for testing or discarded.

Elution 1 and 2. The elution steps were initially carried out following the manufacturer's instructions. 400 μL of the elution buffer was added to the columns and agitated to resuspend the medium. 30 μL of the neutralizing buffer was added to each centrifuge tube. The columns were centrifuged, and the eluate was saved for testing. Although the manufacturer's manual did not specify adding the elution buffer a second time, it did describe carrying out two elution steps. Thus, the elution step procedure was carried out twice. Subsequent optimization of the elution steps for dupilumab and aflibercept are described further below.

Column cleaning. The manufacturer did not provide instructions for column maintenance. For this reason, the column cleaning procedure was developed in which 600 μL of water and 30 μL of the neutralizing buffer were added to each column to neutralize any remaining elution buffer. The column contents were then agitated and centrifuged, and the flowthroughs were discarded. 600 μL of a storage solution consisting of 20% ethanol was then added to each column. The columns were tightly capped on both sides, and the column contents were resuspended by inversion, followed by storage at 4° C.

Titer assay. Cell culture samples and samples purified using the protocol described above in six replicates were tested using a titer assay, measuring absorbance at 280 nm wavelength, in order to measure the protein yield. Samples were optionally filtered under vacuum using the Samplicity® Filtration System prior to analysis using the titer assay.

Protein loss investigation. The previously used columns were investigated to determine if any protein was still present inside, in order to optimize the yield. Storage solution removal, equilibration, and two elution steps were conducted, and the flowthroughs were tested with the titer assay.

Elution and neutralization optimization. For the purification of dupilumab, a series of litmus paper tests was carried out to find an optimal combination of elution buffer and neutralizing buffer. 30 μL, 60 μL, 100 μL and 200 μL of 2 M Tris Base were respectively added to four tubes containing 400 μL of 2.5% acetic acid. Similarly, the pHs of mixtures of the other buffer present in the column manufacturer's buffer kit, 0.1 M glycine, and varying volumes of 1 M and 2 M Tris Base were tested using litmus paper, in order to find the optimal combination. The dupilumab purification protocol was then repeated in duplicate using 400 μL of 0.1 M glycine or 400 μL of 0.24% acetic acid (40 mM acetic acid) as the elution buffer, and 5 μL of 2 M Tris Base as the neutralizing buffer, and the first and/or second eluates were saved for titer testing. 5 μL of 2 M Tris Base was used in the column cleaning step.

The aflibercept purification protocol was optimized to include 400 μL of 0.12% acetic acid (20 mM acetic acid) as the elution buffer, and 30 μL of 2 M Tris Base as the neutralizing buffer. The first eluate was used for testing. 30 μL of 2 M Tris Base was used in the column cleaning step.

Yield optimization. The experiment was repeated using the optimized elution and neutralizing buffers combination. The purified samples to be tested were collected into pre-weighed centrifuge tubes and weighed individually. The density of the samples was assumed to be 1 μg/μL, and the accuracy of the calculations was increased by using actual sample weights instead of theoretical volumes.

Repeatability check. The experiment was repeated according to the improved protocol, using a different centrifuge. The exact eluate weights were used again for an increased calculation accuracy.

Total protein yield measured with UV-visible spectroscopy. Protein loss outside of the binding and washing steps was investigated with UV-visible spectroscopy, aligning the calculation method with established protocols. A commercially available UV-visible spectrophotometer was used to analyze flowthrough samples obtained in the repeatability check run described above. The concentration was measured at 280 nm, using a pre-programmed extinction coefficient characteristic of the protein. The binding eluate samples were excluded from calculations due to the light brown coloration of the samples which caused inaccurate measurements.

Linearity study on column load of dupilumab. The column load range capable of giving consistent, reliable step yields was assessed by purifying varying volumes of dupilumab cell culture samples of known concentration and determining the amount of purified protein by UV-visible spectroscopy.

Theoretical column load values were the target values chosen for the first run of the linearity study (Table 4). The volumes of cell culture added were calculated based on estimated cell culture protein concentration of 8.0 μg/μL. Each eluate was weighed in pre-weighed tubes, giving the exact volume (assuming density to be 1 μg/μL), and the protein concentration was measured using a UV-visible spectrophotometer.

TABLE 4
Column load and volume of buffer values used in the first run of the linearity study.

| Column | 1 | 2 | 3 | 4 | 5 | 6 | 7 | 8 | 9 | 10 |
|---|---|---|---|---|---|---|---|---|---|---|
| Theoretical | 20 | 50 | 100 | 200 | 300 | 400 | 600 | 800 | 1000 | 1200 |
| column load | | | | | | | | | | |
| (μg) | | | | | | | | | | |
| Volume of cell | 2.5 | 6.26 | 12.5 | 25 | 37.6 | 50 | 75 | 100 | 125 | 150 |
| culture sample | | | | | | | | | | |
| (μL) | | | | | | | | | | |
| Volume of | 598 | 594 | 588 | 575 | 563 | 550 | 525 | 500 | 475 | 450 |
| binding/washing | | | | | | | | | | |
| buffer (μL) | | | | | | | | | | |

The linearity experiment was repeated a second time, this time with an additional blank column (Table 5). The antibody purification procedure was followed using a fresh column, with the exception of the antibody binding step, in which 600 UL of binding/washing buffer was added instead of the cell culture sample diluted by the buffer.

TABLE 5
Column load values used in the second run of the linearity study.

| Column | 1 | 2 | 3 | 4 | 5 | 6 | 7 | 8 | 9 | 10 | 11 |
|---|---|---|---|---|---|---|---|---|---|---|---|
| Theoretical | 20 | 50 | 100 | 200 | 300 | 400 | 600 | 800 | 1000 | 1200 | 0 |
| column load | | | | | | | | | | | |
| (μg) | | | | | | | | | | | |
| Volume of cell | 2.5 | 6.26 | 12.5 | 25 | 37.6 | 50 | 75 | 100 | 125 | 150 | 0 |
| culture sample | | | | | | | | | | | |
| (μL) | | | | | | | | | | | |
| Volume of | 598 | 594 | 588 | 575 | 563 | 550 | 525 | 500 | 475 | 450 | 600 |
| binding/washing | | | | | | | | | | | |
| buffer (μL) | | | | | | | | | | | |

Comparison of small-scale to the established large-scale purification method for dupilumab. A dupilumab cell culture sample was purified in three replicates using the improved protocol (76 μL sample per column) and eluates 1 and 2 were combined for each column. The eluate pools and a sample of dupilumab from the same cell culture batch purified using the established large-scale Protein A method were tested for aggregation using size exclusion chromatography (SEC), and the charge variant profile was analyzed using imaged capillary isoelectric focusing electrophoresis (iCIEF) according to standard operating procedures. The purification protocol was then carried out again on another day in order to repeat the aggregation testing.

Aggregation optimization of dupilumab purification method. The purification protocol for dupilumab was further optimized to reduce aggregation by optimizing elution buffers, centrifuge settings, and the neutralization step. All of the samples purified as detailed below were subsequently analyzed using size exclusion chromatography.

To further optimize the elution buffer, aggregate levels of samples eluted using 2.5% acetic acid were compared to samples eluted with 0.1 M glycine, to examine the difference between the two elution buffers from the manufacturer's buffer kit. The eluates obtained from both elution steps were combined for each column.

A different column was used to carry out the same purification protocol, and the binding/washing and elution buffers were the same as in the established large-scale method. Both purification protocols omitted the neutralization step, which was determined to be unnecessary for aggregation testing, as samples do not need to be neutral for size exclusion chromatography.

To further optimize the centrifugation process, the purification protocol was carried out using buffers from the buffer kit, including 0.1 M glycine as the elution buffer. The centrifuging steps were done at 70 RCF and the instrument was manually stopped after 30 seconds during each step. Additionally, three columns were used to carry out the purification protocol using in-house buffers with centrifuge settings reduced to 70 RCF and 30 seconds. The neutralization step was omitted for both methods.

To further optimize the neutralizing buffer, the purification protocol was carried out in triplicate using in-house buffers and the centrifuge set to 1 minute at 100 RCF. 10 or 30 μL of 2 M Tris Base neutralizing buffer (volume determined by litmus tests) was added to each eluate, and either eluate 1 and eluate 2 were combined or eluate 1 was collected for each replicate.

The above experiment was repeated on another day using a different cell culture sample and different batches of the binding/washing, elution, and neutralizing buffers. A sample purified by the established large-scale method from the same cell culture was also analyzed with size exclusion chromatography for comparison. The leftover samples were aliquoted and frozen at −80° C. for further testing.

Charge variant analysis. One aliquot of each previously frozen sample was thawed, and their concentration was measured using UV-visible spectroscopy. Both the spin column-purified and the large-scale-purified samples were divided into two vials, and one half of each sample was dialyzed. The two large-scale-purified samples were diluted to 1.0 mg/mL with water, and the spin column-purified samples were concentrated to 1.0 mg/mL using commercially available centrifugal filters. All four samples were then analyzed with an iCIEF instrument.

Fragmentation analysis. One aliquot of each previously frozen sample was thawed and diluted to 0.3 mg/mL with water. Protein fragmentation was analyzed with capillary electrophoresis with sodium dodecyl sulphate (CE-SDS) using a microchip electrophoresis (MCE) system according to effective standard operating procedures. Samples were treated with sodium dodecyl sulphate (SDS) for both non-reducing and reducing conditions. Reducing conditions were achieved by adding a commercially available reducing agent.

Glycan profile analysis. One aliquot of each previously frozen sample was thawed. The large-scale-purified sample was diluted to 2 mg/mL with water, and the spin column-purified sample was concentrated to 1.8 mg/mL using a centrifugal filter. Their N-glycan profiles (G0F, G1F (1-6), G1F (1-3), and G2F) were then analyzed with hydrophilic interaction chromatography (HILIC) using a commercially available N-Glycan kit according to effective standard operating procedures.

Yield study for the optimized dupilumab purification method. A freshly thawed cell culture harvest pool sample of dupilumab was purified in triplicate into pre-weighed tubes using the optimized method. 81 μL of the sample was added to each column to target the column load of 600 μg, knowing the concentration of the cell culture measured at the time of harvest to be 7.395 mg/mL. Additionally, a blank sample was prepared by carrying out the purification protocol with one change-adding 600 μL of the binding/washing buffer and no cell culture sample during the antibody-binding step.

The eluates obtained after elution 1 and 2 were not combined for any of the four columns. The exact weights of the eluates were recorded, and the concentration of each eluate was measured using UV-visible spectroscopy. The concentrations of blank eluates were subtracted from the purified antibody samples for the respective elution steps. The values were subsequently used to calculate the theoretical incoming protein mass, and the concentrations and exact weights of the eluates were used to calculate the gained protein mass and % yield.

A titer assay was performed on the purified antibody samples and a cell culture sample. The gained protein mass and % yield were calculated in the same way as described above.

Linearity study on column load for optimized purification method of dupilumab and aflibercept. The optimal column load range for the optimized purification method was investigated using both chromatographic and spectroscopic techniques. For the purification method of dupilumab, the column load values were chosen to be 200 μg, 400 μg, 600 μg, 800 μg, and 1000 μg (Table 6). Additionally, blank eluates with a column load of 0 μg were prepared for the spectroscopic measurement of background absorbance. The volumes of cell culture required to achieve the selected column loads were calculated assuming the cell culture concentration to be 6.8 mg/mL, based on historical data.

TABLE 6
Column load and volume of buffer values used
in the linearity study on column load for the
optimized purification method of dupilumab.

| | | | | | | |
|---|---|---|---|---|---|---|
| Theoretical column load (μg) | 200 | 400 | 600 | 800 | 1000 | 0 |
| Volume of cell culture | 29.4 | 58.8 | 88.2 | 117.6 | 147 | 0 |
| sample (μL) | | | | | | |
| Volume of binding/washing | 570.6 | 541.2 | 511.8 | 482.4 | 453 | 600 |
| buffer (μL) | | | | | | |

For the purification method of aflibercept, column load values of 100.5 μg, 201 μg, 402 μg, 603 μg, and 804 μg were evaluated (Table 7). The high column load value (804 μg) was selected based on the maximum load that is typically achieved when using the maximum allowable volume (600 μL) of a harvest material of a typical titer. The lower column loadings were selected to cover low and middle column loading ranges.

TABLE 7
Column load and volume of buffer values used
in the linearity study on column load for the
optimized purification method of aflibercept.

| | | | | | |
|---|---|---|---|---|---|
| Theoretical column load (μg) | 100.5 | 201 | 402 | 603 | 804 |
| Volume of cell culture sample (μL) | 75 | 150 | 300 | 450 | 600 |
| Volume of binding/washing | 525 | 450 | 500 | 150 | 0 |
| buffer (μL) | | | | | |

The optimized purification protocols were carried out in triplicate for the varying column loads except for the one blank column. Eluates obtained in elution steps 1 and/or 2 were collected in separate pre-weighed tubes and their exact weights were recorded. The concentration of each eluate was measured using UV-visible spectroscopy. A cell culture sample and each eluate except the two blank ones were subsequently analyzed by titer assay.

Comparison study of spin column and small-scale purification method for dupilumab. Culture samples were collected at various times. A column load of 600 μg was used for spin column purifications of the mini bioreactor harvest material. Harvest samples were purified in duplicates and 5 μL of 2 M Tris base was used as the neutralization buffer. Details of column loading for the comparison are shown in Table 8.

TABLE 8
Column load and volume of buffer values
used in comparison study of dupilumab.

| Bioreactor | 4 | 5 | 6 | 7 | 8 |
|---|---|---|---|---|---|
| Column Load (μg) | 600 | 600 | 600 | 600 | 600 |
| Volume of cell culture | 96.2 | 95.4 | 92.9 | 92 | 89.5 |
| sample added (μL) | | | | | |
| Volume of binding/ | 503.8 | 504.6 | 507.1 | 508 | 510.5 |
| washing buffer (μL) | | | | | |

Time course sample study for aflibercept. Culture samples were collected at various times. A theoretical column load of 600 μg was used to purify each sample. Column loading was based on the titer of each sample, which varied across the sample set. For the low titer samples, the maximum allowable load volume (600 μL) was used. For higher titer samples, the volume was calculated based on a 600 μg column loading. Purifications were performed in triplicate for time points A, B, C, and D, and a single purification was performed for timepoints, E, F, G, H, I, and J. Details of column loading for the samples are shown in Table 9.

TABLE 9
Column load and volume of buffer values used in time course sample study for aflibercept.

| Timepoint | A | B | C | D | E | F | G | H | I | J | Harvest |
|---|---|---|---|---|---|---|---|---|---|---|---|
| Column Load | 91.8 | 146.4 | 239.4 | 357 | 502 | 600 | 599.4 | 600 | 600 | 600 | 600 |
| (μg) | | | | | | | | | | | |
| Volume of cell | 600 | 600 | 600 | 600 | 600 | 573 | 472 | 431 | 394 | 384 | 407 |
| culture sample | | | | | | | | | | | |
| added (μL) | | | | | | | | | | | |
| Volume of | 0 | 0 | 0 | 0 | 0 | 27 | 128 | 169 | 206 | 216 | 193 |
| binding/washing | | | | | | | | | | | |
| buffer (μL) | | | | | | | | | | | |

### Example 1. Protein Yield Optimization for Dupilumab

Purification of 400 μL of dupilumab cell culture using the initial protocol suggested by the Protein A column manufacturer (with a change in centrifuging time from 30 seconds to 1 minute) resulted in an average 65.3% protein recovery, most likely due to overloading the column. The volume of 400 μL was chosen without knowing the concentration of the cell culture (later measured to be 7.903 μg/μL). As a result, the protein load in the column was approximately 3.16 g, about three times higher than the column's binding capacity listed by the manufacturer.

Two additional elution steps conducted on the same column did not result in a significant amount of eluted protein, suggesting that the average 34.7% of protein lost was not in the column, but rather did not bind to the Protein A in the first place due to binding site unavailability. The protein was likely lost in the flowthroughs of the binding and washing steps.

Decreasing the volume of cell culture used for purification to 76 μL to target a column load of 600 μg resulted in an increase of the protein yield to an average of 73%. This value was deemed unsatisfactory, so the elution and neutralizing buffers were optimized next.

Litmus paper was used to measure the approximate pH values of the elution and neutralizing buffers. The pH of the 2.5% acetic acid elution buffer was found to be ˜2, which was lower than the optimal pH for the elution of antibodies from Protein A (pH 2.5-4) (Chahar et al.; Urh et al.). Moreover, the neutralizing buffer recommended by the column manufacturer was too weakly basic to neutralize the elution buffer to the desired pH of about 7, even when the highest possible volume of 2 M Tris Base (double the recommended molarity), 200 μL, was added to 400 μL of the elution buffer. Following this observation, another elution buffer from the column manufacturer's buffer kit, glycine, was tested with a similar series of litmus tests. The pH of the glycine buffer itself was found to be ˜3 upon diluting the concentrated 1 M glycine to the recommended 0.1 M. This value agreed with the pH given in the buffer kit's manual (pH 2.9). A summary of buffer combinations tested and the measured pH can be seen in Table 10.

TABLE 10
Approximate pH measurements of elution
buffer and Tris Base mixtures.

| | Elution | Tris Base | Tris Base | |
|---|---|---|---|---|
| | buffer | buffer | buffer | Approximate |
| | volume | concentration | volume | pH of |
| Elution buffer | (μL) | (M) | (μL) | mixture |
| 2.5% acetic acid | 400 | 2 | 30 | 3 |
| 2.5% acetic acid | 400 | 2 | 60 | 4 |
| 2.5% acetic acid | 400 | 2 | 100 | 4 |
| 2.5% acetic acid | 400 | 2 | 200 | 5 |
| 0.1M glycine | 400 | 2 | 60 | 10 |
| 0.1M glycine | 400 | 2 | 15 | 9 |
| 0.1M glycine | 400 | 1 | 30 | 9 |
| 0.1M glycine | 400 | 1 | 15 | 8 |
| 0.1M glycine | 400 | 1 | 3 | 3 |
| 0.1M glycine | 400 | 1 | 10 | 7-7.5 |
| 0.1M glycine | 400 | 2 | 5 | 7-7.5 |

The optimal buffer combination was found to be 400 μL of 0.1 M glycine elution buffer and either 10 μL of 1 M Tris Base or 5 μL of 2 M Tris Base neutralizing buffer. 5 μL of 2 M Tris Base was chosen for future experiments due to the availability of 2 M Tris Base, as opposed to 1 M Tris Base, which required an additional dilution step. The pH of 2 M Tris Base was about 10. The protein yield increased to an average of 86.2% when a cell culture sample was purified using this improved buffer combination. The experiment was re-run in six replicates with the accuracy increased by using exact volumes (weighing the eluates and assuming density equal to 1 μg/μL), giving an average yield of 85.7%.

The repeatability of the improved method was then checked by running the experiment on a different day and using a different centrifuge, resulting in an 86.6% yield measured by titer assay. Additionally, the protein concentration of the flowthroughs collected after each centrifuging step was measured using UV-visible spectroscopy (aligning with the large-scale method), and the values were used to calculate an average protein yield of 91.4%, which was comparable to yields obtained using large-scale purification of this antibody.

### Example 2. Column Load Linearity Study of Dupilumab

A linearity study was conducted to determine the ability of the improved method to consistently and reliably produce a high protein yield using various starting protein concentrations. In the first run of the linearity study, a column load range of 20 μg-1200 μg was investigated. The purified protein mass was calculated based on the measured eluate concentration and volume (Table 11), which in turn was calculated from the measured eluate weight, assuming a density of 1 μg/μL. In Table 12, the incoming protein, or the actual column load, was calculated based on the volume of cell culture sample added and the actual cell culture concentration measured by titer assay (7.903 μg/μL). The protein mass obtained in the two elution steps was combined and the % yield was calculated for each column load.

TABLE 11
Measured concentration and volume values for all eluates
and the calculated purified protein mass obtained in
the first run of the linearity study on column load.

| | Theoretical | | | Protein |
|---|---|---|---|---|
| | column load | Concentration | Volume | mass |
| Elution step | (μg) | (μg/μL) | (μL) | (μg) |
| Elution 1 | 20 | 0.05664 | 406.3 | 23.01 |
| | 50 | 0.09033 | 412.3 | 37.24 |
| | 100 | 0.22197 | 409.2 | 90.83 |
| | 200 | 0.42236 | 402.0 | 169.79 |
| | 300 | 0.49961 | 406.0 | 202.84 |
| | 400 | 0.74979 | 404.0 | 302.92 |
| | 600 | 1.20154 | 402.7 | 483.86 |
| | 800 | 1.39268 | 404.6 | 563.48 |
| | 1000 | 1.88324 | 403.6 | 760.08 |
| | 1200 | 2.20543 | 415.8 | 917.02 |
| Elution 2 | 20 | 0.02601 | 404.9 | 10.53 |
| | 50 | 0.04498 | 405.5 | 18.24 |
| | 100 | 0.07511 | 404.8 | 30.40 |
| | 200 | 0.14802 | 406.1 | 60.11 |
| | 300 | 0.29396 | 404.8 | 119.00 |
| | 400 | 0.26590 | 403.0 | 107.16 |
| | 600 | 0.33933 | 407.4 | 138.24 |
| | 800 | 0.50773 | 403.1 | 204.67 |
| | 1000 | 0.71118 | 405.0 | 288.03 |
| | 1200 | 0.62045 | 405.7 | 251.72 |

TABLE 12
Protein yield obtained in the first run of the linearity
study on column load calculated based on actual column
load and the total purified protein mass.

| | | Protein gained | |
|---|---|---|---|
| Theoretical | Incoming | (eluate 1 and 2 | Protein |
| column load | protein | combined) | yield |
| (μg) | (μg) | (μg) | (%) |
| 20 | 19.76 | 33.54 | 169.76 |
| 50 | 49.47 | 55.48 | 112.15 |
| 100 | 98.79 | 121.23 | 122.72 |
| 200 | 197.58 | 229.90 | 116.36 |
| 300 | 297.15 | 321.84 | 108.31 |
| 400 | 395.15 | 410.07 | 103.78 |
| 600 | 592.73 | 622.10 | 104.96 |
| 800 | 790.3 | 768.14 | 97.20 |
| 1000 | 987.88 | 1048.10 | 106.10 |
| 1200 | 1185.45 | 1168.73 | 98.59 |

The protein gained values were higher than the incoming protein values for most replicates, suggesting that other proteins or components of the sample (possibly the glycine buffer) were interfering with the spectroscopy measurements. The linearity study was thus repeated with the addition of a blank sample used to establish the baseline protein concentration. The blank was subtracted from each concentration value and the protein mass was calculated using the exact volumes (Table 13). Actual column load values (Table 14) were calculated based on the actual cell culture sample concentration (7.960 μg/μL) measured on that day by titer assay.

TABLE 13
Measured concentration and volume values for all eluates
and the calculated purified protein mass obtained in
the second run of the linearity study on column load.

| | | | Concen- | | |
|---|---|---|---|---|---|
| | | | tration | | |
| | Theoretical | Concen- | minus blank | | Protein |
| | column load | tration | concentration | Volume | mass |
| Elution step | (μg) | (μg/uL) | (μg/uL) | (μL) | (μg) |
| Elution 1 | 20 | 0.05369 | 0.03249 | 404.0 | 13.13 |
| | 50 | 0.07110 | 0.04990 | 403.6 | 20.14 |
| | 100 | 0.15423 | 0.13303 | 404.0 | 53.74 |
| | 200 | 0.40839 | 0.38719 | 399.5 | 154.68 |
| | 300 | 0.55923 | 0.53803 | 404.9 | 217.85 |
| | 400 | 0.75874 | 0.73754 | 402.6 | 296.93 |
| | 600 | 1.13148 | 1.11028 | 402.9 | 447.33 |
| | 800 | 1.48827 | 1.46707 | 402.1 | 589.91 |
| | 1000 | 1.59393 | 1.57273 | 408.1 | 641.83 |
| | 1200 | 2.02557 | 2.00437 | 403.2 | 808.16 |
| Elution 2 | 20 | 0.03014 | 0.00894 | 400.1 | 3.58 |
| | 50 | 0.04090 | 0.01970 | 408.5 | 8.05 |
| | 100 | 0.07202 | 0.05082 | 405.5 | 20.61 |
| | 200 | 0.10113 | 0.07993 | 400.5 | 32.01 |
| | 300 | 0.16870 | 0.14750 | 405.1 | 59.75 |
| | 400 | 0.21196 | 0.19076 | 405.8 | 77.41 |
| | 600 | 0.31894 | 0.29774 | 396.9 | 118.17 |
| | 800 | 0.43679 | 0.41559 | 404.2 | 167.98 |
| | 1000 | 0.75270 | 0.73150 | 406.8 | 297.57 |
| | 1200 | 0.80943 | 0.78823 | 403.8 | 318.29 |

TABLE 14
Protein yield obtained in the second run of the linearity
study on column load calculated based on actual column
load and the total purified protein mass.

| Theoretical | Incoming | Protein | Protein |
|---|---|---|---|
| column load (μg) | protein (μg) | gained (μg) | yield (%) |
| 20 | 19.9 | 16.70 | 83.93 |
| 50 | 49.8 | 28.19 | 56.57 |
| 100 | 99.5 | 74.35 | 74.73 |
| 200 | 199 | 186.69 | 93.82 |
| 300 | 299.3 | 277.60 | 92.75 |
| 400 | 398 | 374.34 | 94.06 |
| 600 | 597 | 565.50 | 94.72 |
| 800 | 796 | 757.89 | 95.21 |
| 1000 | 995 | 939.41 | 94.41 |
| 1200 | 1194 | 1126.45 | 94.34 |
| 0 | 0 | n/a | n/a |

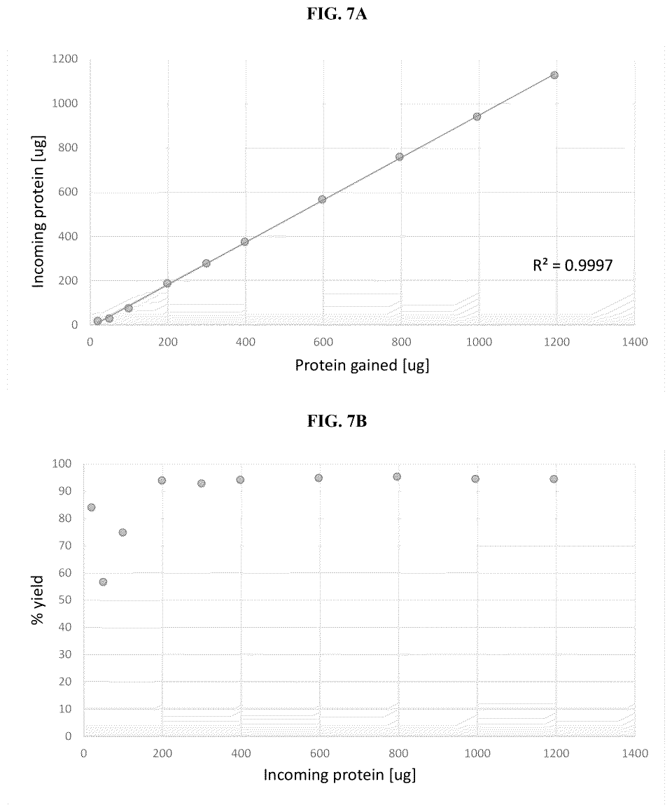

The protein gained was plotted against the incoming protein for each column load, as shown in FIG. 7A, giving a curve with the R2 value equal to 0.9997, showing a highly linear relationship of the two. Subsequently, the protein % yield was plotted against the incoming protein values, as shown in FIG. 7B, and the column load range of 200-1200 μg showed a % yield steadily maintained at around 94.19% (0.76% RSD), while lower column loads resulted in lower % yields, and the % yield for higher column loads remained unknown. The high (94.34%) yield for the 1200 μg column load was unexpected, as the theoretical column capacity provided by the manufacturer was about 1 mg.

### Example 3. Charge Variant and Aggregation Testing of Dupilumab

Dupilumab samples purified in triplicate according to the improved protocol and a dupilumab sample from the same cell culture purified by the large-scale method (comparator sample) were analyzed to compare their product quality attributes.

The dupilumab samples were analyzed for size variation and aggregation using size exclusion chromatography (Table 15). The main peak represents the monomeric form of dupilumab. The HMW peak represents dupilumab with a comparatively high molecular weight, for example aggregates, whereas the LMW peak represents dupilumab with a comparatively low molecular weight, for example fragments. The peak area is directly proportional to the amount of each species in the tested sample. Although the analysis yielded LMW peak area results, this method was directed to quantifying aggregates, and the quantification of fragments was considered unreliable and for information only.

TABLE 15
Size exclusion chromatography results of the triplicate
elution pools and comparator large-scale-purified sample.

| | % HMW | % Main | % LMW |
|---|---|---|---|
| Sample | peak area | peak area | peak area |
| Elution pool 1 | 15.995% | 83.632% | 0.374% |
| Elution pool 2 | 18.599% | 81.024% | 0.377% |
| Elution pool 3 | 15.195% | 84.438% | 0.367% |
| Comparator sample | 8.752% | 91.030% | 0.218% |

The average spin column elution pool aggregation was measured to be 16.596%, about 1.9 times higher than in the large-scale-purified (comparator) sample. This result was deemed unsatisfactory, and the purification protocol was repeated to prepare new samples and test aggregation again (Table 16).

TABLE 16
Size exclusion chromatography results of the triplicate
elution pools and comparator large-scale-purified
sample in the repeated experiment.

| | % HMW | % Main | % LMW |
|---|---|---|---|
| Sample | peak area | peak area | peak area |
| Elution pool 1 | 17.522% | 82.010% | 0.468% |
| Elution pool 2 | 15.972% | 83.554% | 0.475% |
| Elution pool 3 | 16.445% | 83.099% | 0.456% |
| Comparator sample | 8.987% | 90.743% | 0.271% |

The average high molecular weight peak % area for the three elution pool replicates was found to be 16.646%, confirming the previous result of aggregation being ˜1.9 times higher than for the large-scale-purified sample. No subsequent product quality testing was carried out for samples purified using this method. Instead, the cause of high aggregation rates was investigated to optimize the method further and decrease aggregate levels in the small-scale-purified samples.

Aggregation Optimization. Dupilumab samples purified with varying buffers and centrifuge settings were analyzed using size exclusion chromatography. In-house buffers used for the large-scale downstream manufacturing process were tested, including 20 mM sodium phosphate (pH 7.10-7.30) as a binding/washing buffer and 0.24% or 40 mM acetic acid (pH 2.80-3.20) as an elution buffer. Initially, the neutralization step was omitted, as it was thought to be unnecessary for samples being prepared for SEC testing. The results of the optimization experiments are summarized in Table 17 below.

TABLE 17
Summary of SEC results of cell culture samples purified
with varying buffers and centrifuge settings.

| Binding/ | | Neutral- | | | Aggre- | Fragmen- |
|---|---|---|---|---|---|---|
| washing | Elution | izing | Centrifuge | | gation | tation |
| buffer | buffer | buffer | settings | | (%) | (%) |
| TBS | 2.5% | N/A | 1 | minute | 30.2 | 3.1 |
| | acetic acid | | 100 | RCF | | |
| In-house | In-house | N/A | 1 | minute | 6.4 | 1.4 |
| buffer A | buffer B | | 100 | RCF | | |
| TBS | 0.1M | N/A | 30 | seconds | 13.4 | 0.6 |
| | glycine | | 70 | RCF | | |
| In-house | In-house | N/A | 30 | seconds | 6.6-7.3 | 1.1-1.3 |
| buffer A | buffer B | | 70 | RCF | | |
| In-house | In-house | 10 μL | 1 | minute | 6.9-7.3 | 0.5-0.6 |
| buffer A | buffer B | of 2M | 100 | RCF | | |
| | | Tris Base | | | | |

The results showed that returning to the elution buffer used initially, 2.5% acetic acid, almost doubled aggregation to over 30% and increased fragmentation compared to the samples eluted with 0.1 M glycine. When the in-house buffers were used, the aggregation decreased to 6.4% and the fragmentation to 1.4%, the former being comparable to large-scale-purified samples but the latter being undesirably high.

Decreasing the centrifugation time and relative centrifugal force (RCF) decreased aggregation from 16.6% to 13.4% and increased fragmentation from 0.4-0.5% to 0.6% for samples eluted with 0.1 M glycine. While fragmentation decreased slightly from 1.4% to 1.1-1.3% for the samples purified with in-house buffers, the aggregation increased from 6.4% to 6.6-7.3%. It was thus determined that further optimizations in addition to optimizing the centrifuge settings were desirable to improve aggregation and fragmentation.

Subsequently, it was hypothesized that the lack of a neutralizing step could be the cause of lower purity. Dupilumab cell culture samples were thus purified using the in-house buffers again and 10 μL of 2 M Tris Base was added to each eluate. The resulting aggregation (6.9-7.3%) and fragmentation (0.5-0.6%) were both comparable to previous SEC results for large-scale-purified samples.

In order to compare product quality attributes of samples purified from the same cell culture sample, the experiment was repeated using in-house buffers and 10 μL of 2 M Tris Base as the neutralizing buffer. The resulting sample was analyzed using size exclusion chromatography along with a sample purified using the large-scale method on the same day. The results summarized in Table 18 show that the aggregation and fragmentation in the two samples are comparable, with the novel method exhibiting higher purity.

TABLE 18
Comparison of SEC results between samples purified using small-
scale (elution pool) and large-scale (comparator) methods.

| | % HMW | % Main | % LMW |
|---|---|---|---|
| Sample | peak area | Peak area | peak area |
| Elution pool | 6.954% | 92.895% | 0.150% |
| Comparator sample | 9.058% | 90.682% | 0.260% |

Subsequently, the charge variant profiles of a small-scale-purified sample and a large-scale-purified sample were analyzed with iCIEF. Both samples were prepared in two versions-neat and dialyzed-to investigate the effect of diafiltration on the charge variant profile of dupilumab. The results (Table 19) show comparable peak % area values between the dialyzed and neat samples, with the biggest difference of ˜3.5% seen among the acidic variants (region 1) of the large-scale-purified sample. It was thus decided that the additional diafiltration step would be omitted in future analyses.

The results obtained for the spin column-purified and the large-scale-purified samples are also comparable, with spin column-purified region 1 values being within ˜0.28% (neat) or ˜3.2% (dialyzed), and region 2 values being within ˜2.7% (neat) or ˜1.3% (dialyzed) of the large-scale-purified samples. The biggest difference can be seen in the basic charge variants (region 3), which differ by ˜16.1% (neat) and ˜19.1% (dialyzed).

TABLE 19
Comparison of iCIEF results between samples purified using
the new small-scale (elution pool) and established large-
scale (comparator) methods, both dialyzed and neat.

| | Region | Region | Region |
|---|---|---|---|
| Samples | 1 (acidic) | 2 (main) | 3 (basic) |
| Elution pool | 35.7 | 56.5 | 7.8 |
| Dialyzed elution pool | 35.7 | 56.7 | 7.6 |
| Comparator sample | 35.8 | 55.0 | 9.3 |
| Dialyzed comparator | 34.6 | 56.0 | 9.4 |
| sample | | | |

### Example 4. Fragmentation and Glycan Profile Testing of Dupilumab

Aliquots of the same dupilumab samples described in Example 3 were analyzed by CE-SDS under both reducing and non-reducing conditions. The samples were treated with sodium dodecyl sulphate (SDS) to mask the native charges of the proteins and the reducing conditions were achieved by adding a reducing agent to break interchain disulfide bonds, leading to further fragmentation and the appearance of light chain (LC) and heavy chain (HC) peaks on the electropherograms. The results are summarized in Table 20 below (LMW=low molecular weight; NGMP=non-glycosylated main peak; MP=main peak; HMW=high molecular weight).

TABLE 20
Comparison of CE-SDS results between samples purified using the
optimized small-scale method and established large-scale method.

| | Non-reduced | | | | Reduced | | | |
|---|---|---|---|---|---|---|---|---|
| Sample | LMW % | NGMP % | MP % | HMW % | LMW % | NGMP % | MP % | HMW % |
| Elution pool | 6.3826 | 2.6404 | 90.6431 | 0.3340 | 1.0412 | 5.7819 | 91.9563 | 1.2207 |
| Comparator | 5.7922 | 2.0592 | 91.4390 | 0.7096 | 0.5973 | 5.4840 | 92.1616 | 1.3571 |
| sample | | | | | | | | |

Analysis of non-reduced samples provides information about product purity and can be used to assess fragmentation caused by the host cell's proteases, as well as disulfide bond reduction and re-oxidation. The % area of the LMW and HMW peaks represents the amount of low molecular weight species (for example, fragments) and high molecular weight species (for example, aggregates) present in the sample, giving orthogonal information on aggregation in addition to data obtained using size exclusion chromatography. The main peak represents the intact antibody species (Dadouch et al., Separations, 2021, 8:4). The reduced condition allows for the analysis of heavy chain glycosylation occupancy through the quantitation of the non-glycosylated heavy chain, as well as the analysis of the relative amounts of the heavy and light chains (Wagner et al., J. Pharm. Biomed. Anal., 2020, 184:113166).

For the purpose of assessing fragmentation, the LMW peak under the non-reducing conditions was used. The spin column-purified sample exhibited 6.3826% fragmentation, approximately 110% of the large-scale-purified fragmentation (5.7922%). Overall, the parameters were comparable, with the main peak percentage area differing by only 0.87%. There was a significant difference in the non-reduced HMW peaks, with the % area of the spin column-purified sample being less than half of the % area of the large-scale-purified sample. Although this method was not designed for assessing aggregation, this result agrees with the SEC results showing significantly lower aggregate content in the spin column sample.

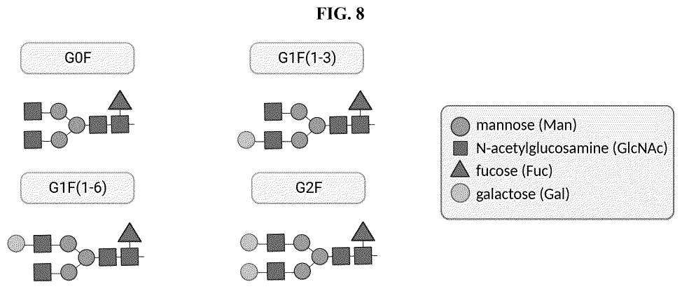

The spin column-purified and the comparator large-scale-purified dupilumab samples were also analyzed with hydrophilic interaction chromatography (HILIC) using an N-Glycan kit in order to assess four N-glycan structures: G0F ((Fuc)1(GlcNAc)2(Man)3+(GlcNAc)2), G1F(1-6) ((Fuc)1(GlcNAc)2(Man)3+(GlcNAc)2 (Gal)1), G1F(1-3) (((Fuc)1(GlcNAc)2(Man)3+(GlcNAc)2(Gal)1), and G2F ((Fuc)1 (GlcNAc)2(Man)3+(GlcNAc)2(Gal)2) and the combined fucosylated glycans (sum of the four N-glycans), as illustrated in FIG. 8. The results are summarized in Table 21.

TABLE 21
Comparison of N-glycan analysis results between
antibody samples purified using new small-scale
and established large-scale methods.

| | Combined | | | | |
|---|---|---|---|---|---|
| | Fucosylated | | | | |
| | Glycans % | G0F | G1F(1-6) | G1F(1-3) | G2F |
| Sample | Area | % Area | % Area | % Area | % Area |
| Spin column | 91.2402 | 68.4915 | 10.0741 | 9.4412 | 3.2334 |
| sample | | | | | |
| Comparator | 90.8094 | 68.1337 | 10.0412 | 9.3567 | 3.2778 |
| sample | | | | | |

The glycan profiles were found to be very similar, with peak % area values being within 0.33-1.35% of one another between the two samples for the four N-glycans and within 0.47% for the combined fucosylated glycans. This final product quality attribute result confirmed that the developed small-scale purification method yields dupilumab with a product quality profile comparable to samples purified using the established large-scale method.

### Example 5. Yield of the Optimized Dupilumab Purification Method

The % yield of a triplicate purification using the optimized developed spin column method was calculated based on two concentration measurement methods—UV-visible spectroscopy and titer assay. The two analyses were carried out on the same eluate samples and on the same day.

In the case of the former technique, a blank sample, prepared by carrying out the purification protocol without adding any cell culture material to the column, was taken as background absorbance and subtracted from the measured concentration of each sample for the respective elution steps. This was done to increase the accuracy of the concentration of the antibody of interest by removing the effect of potential impurities and other sample components absorbing light during the spectroscopic measurements. The exact sample volume was calculated from the eluate weights, assuming a density of 1 mg/mL. The mass of the gained protein was subsequently calculated using the concentration and volume values. The results are summarized in Table 22 below.

TABLE 22
Concentration of spin column eluates measured with UV-visible
spectroscopy and the calculated protein mass gained.

| | Concentration | | | |
|---|---|---|---|---|
| | (μg/μL) | Conc. - | | Protein |
| | (UV-visible | blank conc. | Volume | gained |
| Sample | spectroscopy) | (μg/uL) | (μL) | (μg) |
| Blank elution 1 | 0.05496 | 0 | 403.5 | 0 |
| Column 1 elution 1 | 0.90925 | 0.85429 | 402.8 | 344.108 |
| Column 2 elution 1 | 0.78190 | 0.72694 | 403.7 | 293.466 |
| Column 3 elution 1 | 0.77711 | 0.72215 | 402.7 | 290.810 |
| Blank elution 2 | 0.01267 | 0 | 405.7 | 0 |
| Column 1 elution 2 | 0.38293 | 0.37026 | 405.2 | 150.029 |
| Column 2 elution 2 | 0.47666 | 0.46399 | 397.9 | 184.622 |
| Column 3 elution 2 | 0.49872 | 0.48605 | 407.2 | 197.920 |

The mass of protein gained in the two elution steps was then summed for each column. The incoming protein mass was calculated based on the volume of cell culture added to each column (81 μL) and concentration measured by titer assay on the day of the purification (6.362 mg/mL). The incoming protein and gained protein values were then used to calculate the % yield of the purification step for each column, averaging at 94.50%, as shown in Table 23.

TABLE 23
Percentage yield of the developed protein purification method for
each of the triplicate runs according to gained protein mass calculated
from concentration obtained with UV-visible spectroscopy.

| | Protein gained | Incoming | |
|---|---|---|---|
| Column | Elution 1 + 2 (μg) | protein (μg) | % Yield |
| 1 | 494.137 | 515.322 | 95.89% |
| 2 | 478.087 | 515.322 | 92.77% |
| 3 | 488.729 | 515.322 | 94.84% |
| Average | 486.985 | | 94.50% |

No blank sample was required for the titer assay. The obtained concentration values and the exact sample volumes were used to calculate the mass of protein gained (Table 24).

TABLE 24
Concentration of spin column eluates measured by titer
assay and the calculated protein mass gained.

| | Concentration | | Protein |
|---|---|---|---|
| | (μg/μL) | Volume | gained |
| Sample | (titer assay) | (μL) | (μg) |
| Column 1 elution 1 | 0.835 | 402.8 | 336.338 |
| Column 2 elution 1 | 0.718 | 403.7 | 289.857 |
| Column 3 elution 1 | 0.744 | 402.7 | 299.609 |
| Column 1 elution 2 | 0.367 | 405.2 | 148.708 |
| Column 2 elution 2 | 0.455 | 397.9 | 181.045 |
| Column 3 elution 2 | 0.458 | 407.2 | 186.498 |

Similarly to the spectroscopic results, the protein mass was summed for each replicate and the previously calculated incoming protein value was used to determine the % yield (Table 25).

TABLE 25
Percentage yield of the developed protein purification method
for each of the triplicate runs according to gained protein
mass calculated from concentration measured with titer assay.

| | Protein gained | |
|---|---|---|
| Column | Elution 1 + 2 (μg) | % Yield |
| 1 | 485.046 | 94.12% |
| 2 | 470.901 | 91.38% |
| 3 | 486.106 | 94.33% |
| Average | 480.685 | 93.28% |

The two average % yield values calculated using spectroscopic and chromatographic techniques, 94.50% and 93.28%, are comparable to one another, as well as to the step yields obtained using the established large-scale purification method. This confirms that the novel small-scale method delivers purified dupilumab with both a similar yield and PQ profile.

### Example 6. Column Load of the Optimized Dupilumab Purification Method

Protein % yield for dupilumab purification carried out in triplicate using varying theoretical column loads (200 μg, 400 μg, 600 μg, 800 μg and 1000 μg) was calculated using eluate concentration values obtained with UV-visible spectroscopy and by titer assay in order to assess the optimal column load range. Similarly to the yield study described in Example 5, a blank sample was used to measure the background absorbance for the spectroscopic measurements and the exact volumes of the eluates were determined by measuring the exact eluate weights, assuming density to be 1 mg/mL. See results summarized in Table 26.

TABLE 26
Concentration of spin column eluates measured with UV-visible
spectroscopy and the calculated protein mass gained.

| | Concentration | Conc. - | | |
|---|---|---|---|---|
| | (μg/μL) | blank | Vol- | Protein |
| | (UV-visible | conc. | ume | gained |
| Sample | spectroscopy) | (μg/μL) | (μL) | (μg) |
| Blank elution 1 | 0.01702 | N/A | N/A | N/A |
| Blank elution 2 | 0.01668 | N/A | N/A | N/A |
| Replicate 1 elution 1 200 μg | 0.29307 | 0.27605 | 408.0 | 112.628 |
| Replicate 1 elution 1 400 μg | 0.51600 | 0.49898 | 410.3 | 204.731 |
| Replicate 1 elution 1 600 μg | 0.83008 | 0.81306 | 408.0 | 331.728 |
| Replicate 1 elution 1 800 μg | 1.27299 | 1.25597 | 419.5 | 526.879 |
| Replicate 1 elution 1 1000 μg | 1.54595 | 1.52893 | 397.3 | 607.444 |
| Replicate 2 elution 1 200 μg | 0.23508 | 0.21806 | 408.9 | 89.165 |
| Replicate 2 elution 1 400 μg | 0.53656 | 0.51954 | 417.1 | 216.700 |
| Replicate 2 elution 1 600 μg | 0.71963 | 0.70261 | 409.0 | 287.367 |
| Replicate 2 elution 1 800 μg | 1.05740 | 1.04038 | 413.0 | 429.677 |
| Replicate 2 elution 1 1000 μg | 1.38903 | 1.37201 | 408.7 | 560.740 |
| Replicate 3 elution 1 200 μg | 0.21884 | 0.20182 | 411.7 | 83.089 |
| Replicate 3 elution 1 400 μg | 0.54754 | 0.53052 | 408.7 | 216.824 |
| Replicate 3 elution 1 600 μg | 0.84039 | 0.82337 | 420.8 | 346.474 |
| Replicate 3 elution 1 800 μg | 1.38314 | 1.36612 | 412.7 | 563.798 |
| Replicate 3 elution 1 1000 μg | 1.33888 | 1.32186 | 406.1 | 536.807 |
| Replicate 1 elution 2 200 μg | 0.15833 | 0.14165 | 416.2 | 58.955 |
| Replicate 1 elution 2 400 μg | 0.36808 | 0.35140 | 413.8 | 145.409 |
| Replicate 1 elution 2 600 μg | 0.52701 | 0.51033 | 415.0 | 211.787 |
| Replicate 1 elution 2 800 μg | 0.45674 | 0.44006 | 408.4 | 179.721 |
| Replicate 1 elution 2 1000 μg | 0.60212 | 0.58544 | 410.4 | 240.265 |
| Replicate 2 elution 2 200 μg | 0.19760 | 0.18092 | 406.6 | 73.562 |
| Replicate 2 elution 2 400 μg | 0.36748 | 0.35080 | 412.4 | 144.670 |
| Replicate 2 elution 2 600 μg | 0.54700 | 0.53032 | 413.6 | 219.340 |
| Replicate 2 elution 2 800 μg | 0.68818 | 0.67150 | 408.3 | 274.173 |
| Replicate 2 elution 2 1000 μg | 0.74929 | 0.73261 | 409.2 | 299.784 |
| Replicate 3 elution 2 200 μg | 0.20323 | 0.18655 | 413.2 | 77.082 |
| Replicate 3 elution 2 400 μg | 0.37093 | 0.35425 | 413.4 | 146.447 |
| Replicate 3 elution 2 600 μg | 0.50109 | 0.48441 | 410.0 | 198.608 |
| Replicate 3 elution 2 800 μg | 0.38341 | 0.36673 | 406.3 | 149.002 |
| Replicate 3 elution 2 1000 μg | 0.75887 | 0.74219 | 411.8 | 305.634 |

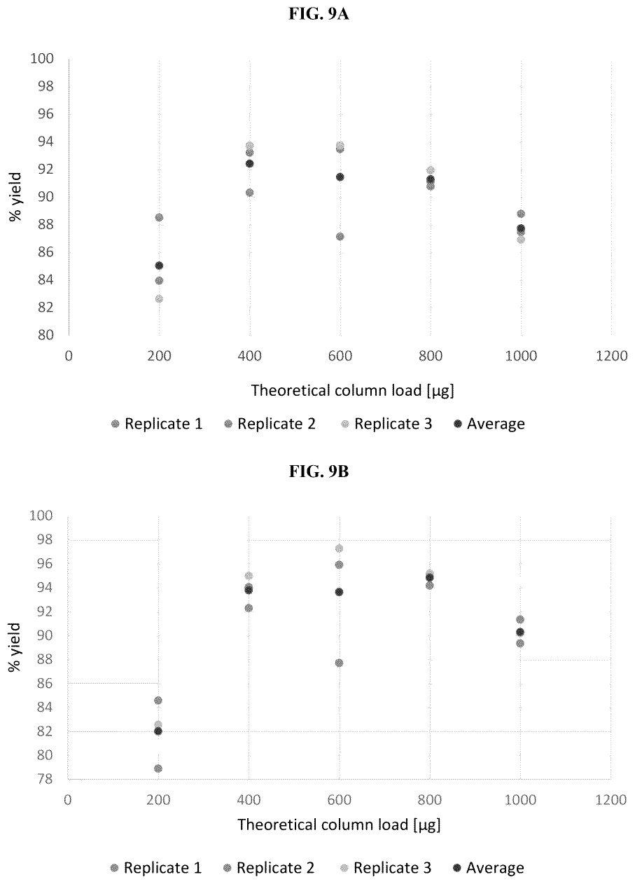

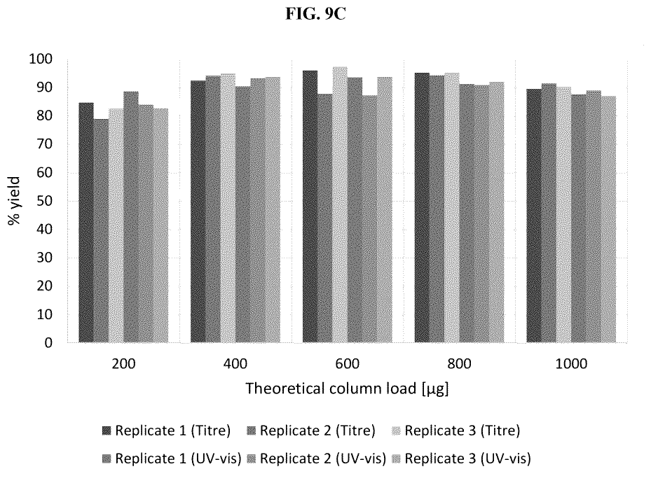

The protein mass gained in each elution step was summed for each replicate. The incoming protein mass was calculated based on the volume of cell culture added to each column and its concentration measured by titer assay (6.594 mg/mL). Subsequently, the incoming protein and the gained protein values were used to calculate the % yield for each column, as summarized in Table 27 and FIG. 9A.

TABLE 27
Percentage yield of the developed protein purification method for
each of the triplicate runs according to gained protein mass calculated
from concentration obtained with UV-visible spectroscopy.

| | Protein gained | Cell culture | Incoming | |
|---|---|---|---|---|
| | Elution 1 + 2 | sample added | protein | % |
| Column | (μg) | (μL) | (μg) | Yield |
| Replicate 1 200 μg | 171.58313 | 29.4 | 193.864 | 88.51 |
| Replicate 1 400 μg | 350.140814 | 58.8 | 387.727 | 90.31 |
| Replicate 1 600 μg | 543.51543 | 88.2 | 581.591 | 93.45 |
| Replicate 1 800 μg | 706.599919 | 117.6 | 775.454 | 91.12 |
| Replicate 1 1000 μg | 847.708465 | 147 | 969.318 | 87.45 |
| Replicate 2 200 μg | 162.726806 | 29.4 | 193.864 | 83.94 |
| Replicate 2 400 μg | 361.370054 | 58.8 | 387.727 | 93.20 |
| Replicate 2 600 μg | 506.707842 | 88.2 | 581.591 | 87.12 |
| Replicate 2 800 μg | 703.85039 | 117.6 | 775.454 | 90.77 |
| Replicate 2 1000 μg | 860.524499 | 147 | 969.318 | 88.78 |
| Replicate 3 200 μg | 160.171754 | 29.4 | 193.864 | 82.62 |
| Replicate 3 400 μg | 363.270474 | 58.8 | 387.727 | 93.69 |
| Replicate 3 600 μg | 545.082196 | 88.2 | 581.591 | 93.72 |
| Replicate 3 800 μg | 712.800123 | 117.6 | 775.454 | 91.92 |
| Replicate 3 1000 μg | 842.441188 | 147 | 969.318 | 86.91 |

Eluate concentration was also measured by titer assay and the gained protein mass (Table 28) was calculated according to the exact eluate weights. The protein gained in the two elution steps was summed for each column and the % yield was calculated using the previously determined incoming protein values, as shown in Table 29 and FIG. 9B.

TABLE 28
Concentration of spin column eluates measured by titer
assay and the calculated protein mass gained.

| | Concentration | | Protein |
|---|---|---|---|
| | (μg/μL) | Volume | gained |
| Sample | (titer assay) | (μL) | (μg) |
| Replicate 1 elution 1 200 μg | 0.259 | 408.0 | 105.672 |
| Replicate 1 elution 1 400 μg | 0.508 | 410.3 | 208.432 |
| Replicate 1 elution 1 600 μg | 0.839 | 408.0 | 342.312 |
| Replicate 1 elution 1 800 μg | 1.321 | 419.5 | 554.160 |
| Replicate 1 elution 1 1000 μg | 1.572 | 397.3 | 624.556 |
| Replicate 2 elution 1 200 μg | 0.198 | 408.9 | 80.962 |
| Replicate 2 elution 1 400 μg | 0.532 | 417.1 | 221.897 |
| Replicate 2 elution 1 600 μg | 0.714 | 409.0 | 292.026 |
| Replicate 2 elution 1 800 μg | 1.087 | 413.0 | 448.931 |
| Replicate 2 elution 1 1000 μg | 1.432 | 408.7 | 585.258 |
| Replicate 3 elution 1 200 μg | 0.201 | 411.7 | 82.752 |
| Replicate 3 elution 1 400 μg | 0.544 | 408.7 | 222.333 |
| Replicate 3 elution 1 600 μg | 0.859 | 420.8 | 361.467 |
| Replicate 3 elution 1 800 μg | 1.430 | 412.7 | 590.161 |
| Replicate 3 elution 1 1000 μg | 1.375 | 406.1 | 558.388 |
| Replicate 1 elution 2 200 μg | 0.140 | 416.2 | 58.268 |
| Replicate 1 elution 2 400 μg | 0.361 | 413.8 | 149.382 |
| Replicate 1 elution 2 600 μg | 0.519 | 415.0 | 215.385 |
| Replicate 1 elution 2 800 μg | 0.449 | 408.4 | 183.372 |
| Replicate 1 elution 2 1000 μg | 0.588 | 410.4 | 241.315 |
| Replicate 2 elution 2 200 μg | 0.177 | 406.6 | 71.968 |
| Replicate 2 elution 2 400 μg | 0.346 | 412.4 | 142.690 |
| Replicate 2 elution 2 600 μg | 0.527 | 413.6 | 217.967 |
| Replicate 2 elution 2 800 μg | 0.689 | 408.3 | 281.319 |
| Replicate 2 elution 2 1000 μg | 0.733 | 409.2 | 299.944 |
| Replicate 3 elution 2 200 μg | 0.187 | 413.2 | 77.268 |
| Replicate 3 elution 2 400 μg | 0.353 | 413.4 | 145.930 |
| Replicate 3 elution 2 600 μg | 0.498 | 410.0 | 204.180 |
| Replicate 3 elution 2 800 μg | 0.364 | 406.3 | 147.893 |
| Replicate 3 elution 2 1000 μg | 0.767 | 411.8 | 315.851 |

TABLE 29
Percentage yield of the developed protein purification method
for each of the triplicate runs according to gained protein
mass calculated from concentration measured by titer assay.

| | Protein gained | Incoming | |
|---|---|---|---|
| Column | Elution 1 + 2 (μg) | protein (μg) | % Yield |
| Replicate 1 200 μg | 163.940 | 193.864 | 84.56 |
| Replicate 1 400 μg | 357.814 | 387.727 | 92.29 |
| Replicate 1 600 μg | 557.697 | 581.591 | 95.89 |
| Replicate 1 800 μg | 737.531 | 775.454 | 95.11 |
| Replicate 1 1000 μg | 865.871 | 969.318 | 89.33 |
| Replicate 2 200 μg | 152.930 | 193.864 | 78.89 |
| Replicate 2 400 μg | 364.588 | 387.727 | 94.03 |
| Replicate 2 600 μg | 509.993 | 581.591 | 87.69 |
| Replicate 2 800 μg | 730.250 | 775.454 | 94.17 |
| Replicate 2 1000 μg | 885.202 | 969.318 | 91.32 |
| Replicate 3 200 μg | 160.020 | 193.864 | 82.54 |
| Replicate 3 400 μg | 368.263 | 387.727 | 94.98 |
| Replicate 3 600 μg | 565.647 | 581.591 | 97.26 |
| Replicate 3 800 μg | 738.054 | 775.454 | 95.18 |
| Replicate 3 1000 μg | 874.238 | 969.318 | 90.19 |

Both the spectroscopic and the chromatographic analytical techniques show that the optimal column load range with the average yield >90% is 400-800 μg. A comparison of the results obtained by the two methods can be seen in FIG. 9C. The 600 μg column achieved the highest yields (93.72% by UV-visible spectroscopy and 97.26% by titer assay), although the average yield for that column load is lowered by the significantly lower yield in replicate 2 (87.12% for UV-visible spectroscopy and 87.69% for titer assay), likely caused by sample loss occurring before the eluates were weighed, e.g. the eluate being spilled during the purification procedure or sample transport. However, it is worth noting that the purification method is developed for PQ testing purposes, therefore maximizing the yield further is not a priority.

### Example 7. Comparison of Novel Small-Scale and Established Large-Scale Methods for Purifying Dupilumab

The experimental results disclosed in Examples 1-6 show that dupilumab purified from upstream CHO cell culture material using the small-scale method exhibits a comparable PQ profile and step yield relative to dupilumab samples purified using the established large-scale method. Both techniques utilize Protein A affinity chromatography, with the novel method using small-scale spin columns and the established large-scale method employing traditional liquid chromatography systems.

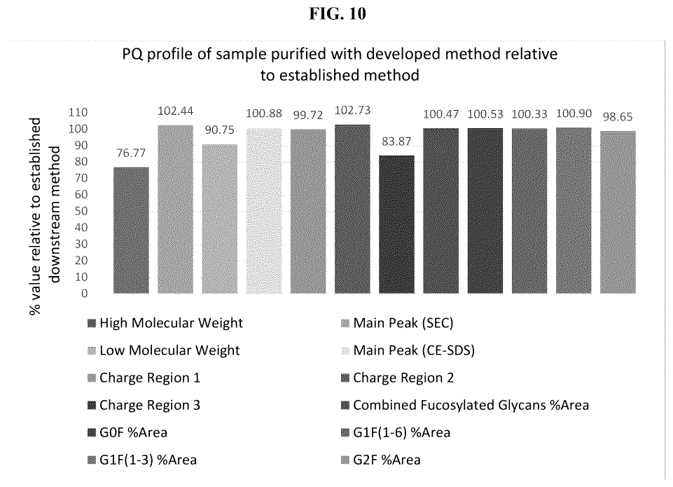

Cell culture samples from the same batch purified using the two methods on the same day were analyzed in terms of their critical product quality attributes: aggregation, fragmentation, charge variants and glycan profile. A summary of the results is shown in FIG. 10.

It was found that the product quality results of the spin column-purified sample were within 1% of the large-scale-purified sample in terms of the overall combined fucosylated glycans and all N-glycan structures (G0F, G1F(1-6), G1F(1-3)) except for the G2F glycoform, which was within 1.4%. The results suggest that the glycan profiles of samples purified using these two methods are comparable.

The charge variant profile of the spin column-purified sample was also found to be comparable, with the acidic variants (region 1) being within 0.3% and the “neutral” variants (variants with a similar pI value, region 2) being within 2.8% of the large-scale-purified sample. The basic variants (region 3) peak area was found to be about 84% relative to the established large-scale method; however, the relatively large difference was not unexpected. This particular antibody's basic variants are low in abundance, resulting in small peak areas and high assay variability. A similar variability is seen among the standard injections, and as a result the standard operating procedure of iCIEF analysis has no % RSD requirements for region 3 values, as opposed to the other two regions. Furthermore, the percentage difference between the normalized data is relatively large due to the actual % area values it compares being much smaller than for the other two regions, while the numerical difference between the actual region 3% area values is not large.

The spin column-purified samples exhibited somewhat lower fragmentation (90.75%) and aggregation (76.77%) results, meaning that they had a relatively slightly different purity than the large-scale-purified samples. This was a surprising result, as it was expected that the multiple centrifugation steps of the small-scale method could potentially increase aggregation rates due to mechanical stress. However, the results suggest that either less fragmentation and aggregation occurred during the purification process itself, potentially due to the difference in exposure to low pH conditions, or that the small-scale method is able to remove those impurities, which may depend on the type of Protein A resin in the columns used. In the small-scale method, the two elution steps took less than 5 minutes in total, and the eluates were immediately neutralized with the neutralizing buffer. In the large-scale purification method, the elution of the antibody from the chromatographic column took more time, and the eluates were not neutralized, as acidic conditions were required for the viral inactivation step, which follows affinity chromatography in the downstream purification process. The amplifying effect of low pH conditions on the rates of aggregation and fragmentation has been described above (Jin et al.; Hu et al.). The subsequent purification steps in large-scale purification further polished the product produced, removing additional impurities that arise during the downstream manufacturing process. In the future, this hypothesis could be investigated by carrying out the small-scale purification protocol as usual, with the exception of aligning the amount of time during which the sample is exposed to each buffer with the timeline of the established large-scale method.

### Example 8. Comparison of Spin Column and Small-Scale Purification Method of Dupilumab

Different small bioreactor harvest samples of dupilumab having similar titers were purified using spin columns. Following purification, the samples were analyzed for protein concentration, aggregation, fragmentation, charge variants and glycan profile. The aim of this study was to determine if the optimized spin column method for purifying dupilumab could be used to purify harvest samples obtained from small bioreactors having a volume of just a few hundred milliliters.

For the purpose of data analysis, all values obtained from spin column purified samples were normalized to values obtained from routine small-scale purified samples. The normalized value is the percentage comparability with 100% being fully comparable and values above or below 100% representing the percent change from values obtained from small-scale purification. Table 8 shows the percentage comparability of aggregation and charge between samples purified using the new small-scale spin column purification method and established small-scale purification method.

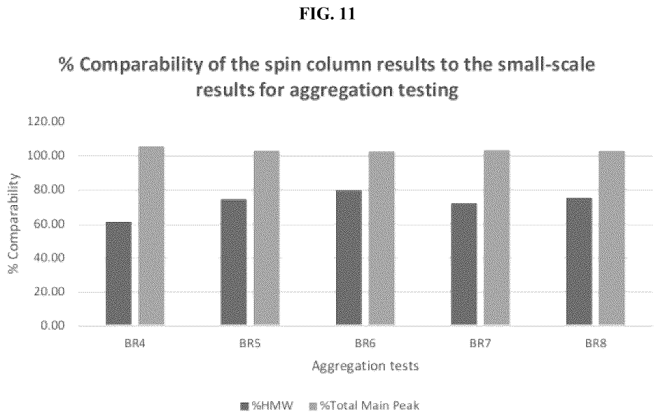

The percent comparability of HMW species between the spin column purified samples and the small-scale purified samples ranged from −38.88% to −20.62%. These results suggest that the spin column purified sample exhibited lower % HMW than the small-scale purified sample (Table 30 and FIG. 11). Further, the percent comparability of total main species between the spin column purified samples and the small-scale purified samples ranged from +2.41% to +5.30%, suggesting that the purity of the spin column purified samples were comparable to that of the small-scale purified samples. (Table 30 and FIG. 11).

TABLE 30
Comparison of CE-SDS and icIEF results between samples purified
using new spin column and established small-scale methods.

| | Comparability (%) | | | | |
|---|---|---|---|---|---|
| | Aggregation | | | | |
| | | % Total | Charge | | |
| Bioreactor | % HMW | Main Peak | Region 1 | Region 2 | Region 3 |
| BR4 | 61.12 | 105.30 | 109.39 | 99.24 | 72.97 |
| BR5 | 74.47 | 102.79 | 108.97 | 95.54 | 90.43 |
| BR6 | 79.38 | 102.41 | 106.49 | 97.74 | 87.88 |
| BR7 | 71.84 | 103.10 | 109.12 | 97.61 | 79.79 |
| BR8 | 75.19 | 102.67 | 103.52 | 102.24 | 73.68 |

The discrepancy in the percent comparability of HMW species was attributed to the fact that the small-scale samples were post-viral inactivated samples. Viral inactivation is a process whereby the protein A eluate is exposed to a lower pH environment for the purpose of inactivating viruses. This process could cause the proteins to aggregate and may explain why the spin column purified samples, which do not undergo viral inactivation, exhibit lower levels of aggregation.

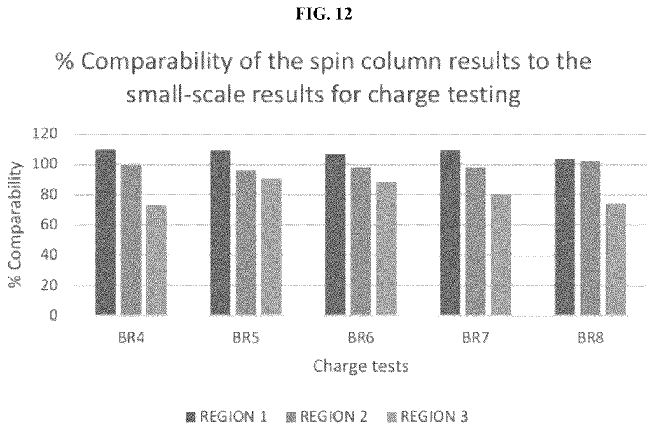

The charge variant profile of the spin column purified and the small-scale purified samples was found to vary depending on the region (Table 30 and FIG. 12). For example, the comparability results among the acidic variants (region 1) ranged from +3.52% to +9.39%, whereas the main species (region 2) ranged from −4.46% to +2.24% and basic variants (region 3) ranged from −27.03% to −9.57%. Samples from bioreactor 5 and 6 had the most similar charge variant profile across all regions, particularly region 3.

The potential explanations for bioreactors 4, 7, and 8 being less comparable for region 3 than bioreactors 5 and 6 were assessed by analyzing the obtained concentrations for each purification, the comparability between the small-scale values, conditions in which the experimentation was performed, and incubation time. However, the reason could not be determined. Further, it has been previously observed that the charge variant profile of region 3 showed high variability.

It must be noted that the comparability results listed above were normalized, which caused significant variability. However, the variability between the actual values was small. Table 31 shows an example of the difference in the actual values for region 3, in which the percent difference in charge between the spin column purified samples and small-scale purified samples ranged from 0.9% for bioreactor 5 to 3.0% for bioreactor 4. However, when these values were normalized, the percentage comparability in charge for region 3 was 90.43% for bioreactor 5 and 72.97% for bioreactor 4, highlighting the difference between the actual and normalized values.

TABLE 31
Percentage difference in charge values obtained for region 3 between
spin column purified samples and small-scale purified samples.

| Bioreactor | Region 3 (% difference) |
|---|---|
| BR4 | 3.0 |
| BR5 | 0.9 |
| BR6 | 1.2 |
| BR7 | 1.9 |
| BR8 | 2.5 |

To assess fragmentation, the low molecular weight species (LMW) of the spin column and small-scale purified samples under non-reducing conditions were analyzed (Wong et al., 2023, Analytical Biochemistry, 666:115073). These low molecular weight species included impurities of the product or process caused by cellular enzymes, re-oxidation, and reduction of disulfide bonds (Dadouch et al., 2021, Separations, 8 (1): 4). The samples under reducing conditions were used to determine the number of non-glycosylated heavy chains, low molecular weight, and high molecular weight species (Dadouch et al.). As shown in Table 32, the percentage comparability of LMW species between the spin column purified samples and the small-scale purified samples under non-reducing conditions ranged from −21.91% to −10.69% across all bioreactors. Under reducing conditions, the percentage comparability of LMW between the spin column purified samples and the small-scale purified samples ranged from −36.78% to −7.02%.

TABLE 32
Fragmentation analysis of spin column purified samples and
small-scale purified samples from different bioreactors.

| | Comparability (%) | | | | | |
|---|---|---|---|---|---|---|
| | Non-reduced | | | Reduced | | |
| | | | Purity % | | | Purity % |
| Bioreactor | LMW % | NGMP % | (MP) | LMW % | NGHC % | (LC + HC)% |
| BR4 | 89.31 | 99.98 | 100.92 | 92.98 | 93.72 | 100.62 |
| BR5 | 85.48 | 102.11 | 101.41 | 91.77 | 96.48 | 100.65 |
| BR6 | 78.79 | 92.91 | 101.74 | 90.80 | 100.43 | 100.51 |
| BR7 | 78.09 | 110.27 | 101.60 | 63.22 | 97.21 | 100.80 |
| BR8 | 85.35 | 104.14 | 101.03 | 74.96 | 98.75 | 101.09 |

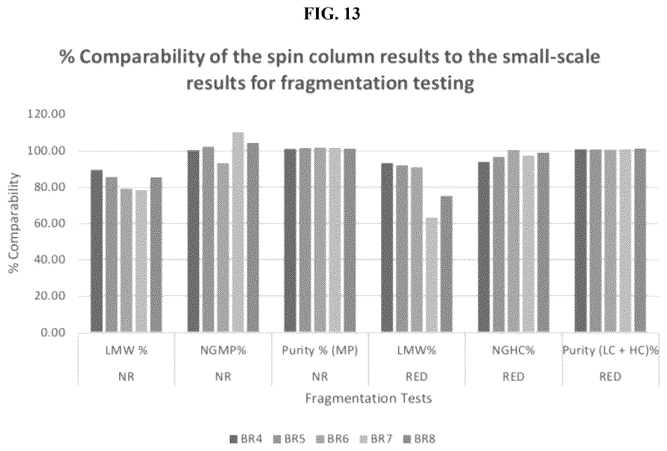

As shown in FIG. 13, the spin column purified samples exhibited comparable % purity, % NGMP (non-glycosylated main peak), and % NGHC (non-glycosylated heavy chain) to the small-scale purified samples under both reduced and non-reduced conditions. Interestingly, the spin column purified samples exhibited lower % LMW under both non-reduced and reduced conditions. This could be due to the impact of the low pH on the samples during viral inactivation, which could have led to the increased fragmentation in the small-scale purified samples. Indeed, in a study which was carried out to investigate the effects of different pH and buffer species on monoclonal antibodies, it was demonstrated that lower pH conditions cause more fragmentation than higher pH conditions (Zheng et al., 2015, AAPS PharmaSciTech, 18 (1): 42-48). The lower pH was found to unfold the CH2 domain, leading to increased surface accessibility and fragmentation (Zheng et al.).

Together, these results demonstrate that comparable product quality results can be obtained between the spin column purified and small-scale purified samples, suggesting that the small-scale purification method for dupilumab gives comparable results when using harvest material from a mini bioreactor.

### Example 9. Optimization of Purification Method for Aflibercept

In order to develop an optimal purification method for aflibercept, aflibercept samples purified using the spin column method and a routine small-scale column method were analyzed to compare their product quality attributes. Two elution steps were carried out for the spin column method using the same elution buffer with the second elution step performed to maximize yield. As the same elution buffer was used for both elution steps, eluate 1 and 2 were considered to be comparable in terms of protein population. For the purpose of carrying out product quality testing, only eluate 1 was analyzed as combining the two eluates would result in a diluted sample.

Initially, the product quality attributes of aflibercept purified using the spin column method with the buffers provided in the manufacturer's manual was compared to that of aflibercept purified using the small-scale purification method with in-house buffers. The spin column was loaded with 603 μg of aflibercept harvest material and purification was performed using manufacturer's buffer. The eluate was then analyzed by size exclusion ultra performance liquid chromatography (SE-UPLC) to assess purity and aggregation levels, and by imaged capillary isoelectric focusing (iCIEF) for charge profile characterization.

Table 33 shows the percentage comparability of aggregation and charge between samples obtained from spin column purification using the manufacturer's buffers and samples obtained from routine small-scale purification using in-house buffers. Differences in aggregation and charge profile were observed between the spin column purified sample and small-scale purified sample. Results from SE-UPLC analysis show that the total main peak of the spin column purified sample was 44.9% lower compared to the small-scale purified sample. Further, the % HMW species was found to be 2567% higher in the small column purified sample than that of the small-scale purified sample. This indicates that a considerable quantity of the spin column eluate had molecules that aggregated, which was not desirable in terms of product quality. The low molecular weight results were disregarded as the technique used for the aggregation experimentation specifically measured high molecular weight species and total mean peak, and thus, the low molecular weight results were not considered to be accurate.

Differences in the charge profiles were observed for all three regions, with spin column-purified values being 40.75% lower for region 1, 24.85% higher for region 2, and 9.19% higher for region 3 than small-scale purified values. Therefore, it was concluded that there was a significant difference in product quality attributes between samples obtained using the routine small-scale purification method and the spin column purification method, which used the column manufacturer's buffers. This indicates that the spin column method using the manufacturer's buffers was unsuitable for purifying aflibercept.

TABLE 33
Comparison of CE-SDS and icIEF results between samples
purified using spin column method using manufacturer's
buffers and small-scale method using in-house buffers.

Aggregation

| | |
|---|---|
| % Total | Charge |

| % HMW | Main Peak | Region 1 | Region 2 | Region 3 |
|---|---|---|---|---|
| 2667 | 55.1 | 59.25 | 124.85 | 109.19 |

Since the results obtained with the spin column purification method using the manufacturer's buffers were unsatisfactory when compared to the established small-scale method, the spin column method using in-house buffers was assessed. The spin column was loaded with 603 μg of aflibercept harvest material, and the aggregation and purity levels and charge profile of the eluate was assessed. Table 34 shows the percentage comparability of aggregation and charge between samples obtained from spin column purification using in-house buffers and samples obtained from routine small-scale purification. The results show that the aggregation and charge of the spin column purified sample was comparable to the small-scale purified sample. The total main peak of the spin column purified sample was found to be 0.08% higher than the small-scale purified sample. Moreover, the % HMW of the spin column purified sample was found to be 4.4% lower than the small-scale purified sample, which indicates lower aggregation in the aflibercept sample obtained using the new spin column method.

In terms of the charge profile, region 2, which consists of the main or neutral species, was found to be 3.85% higher in the spin column purified sample compared to the small-scale purified sample, with the difference being only a few percent. Further, the percentage comparability of region 1 and 3 in the spin column purified sample was 11.99% higher and 12.97% lower than the small-scale purified sample. Although the spin column values and small-scale values differed to some extent, the difference was considered negligible and the values were deemed comparable. Together, these results highlight the suitability of the in-house buffers for use with the spin column purification method for aflibercept.

TABLE 34
Comparison of CE-SDS and icIEF results between
samples purified using spin column method using
in-house buffers and small-scale method.

Aggregation

| | |
|---|---|
| % Total | Charge |

| % HMW | Main Peak | Region 1 | Region 2 | Region 3 |
|---|---|---|---|---|
| 95.6 | 100.8 | 111.99 | 103.85 | 87.03 |

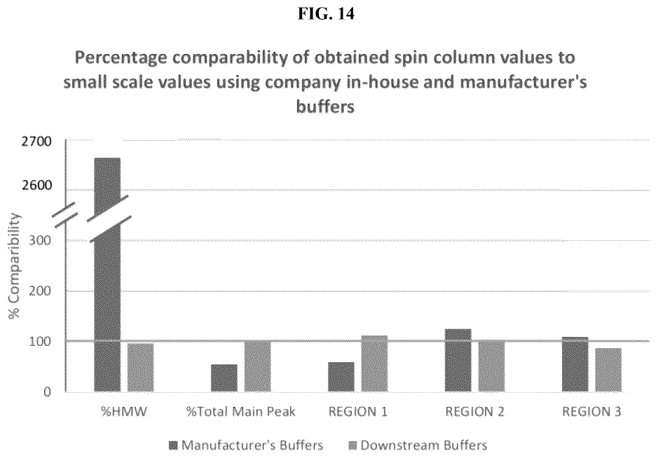

As shown in FIG. 14, there was a stark difference in the product quality values obtained using the in-house buffers compared to the manufacturer's buffers. There was also a similarity between the values obtained from the small-scale purification and the spin column purification using in-house buffers. These results demonstrate that the in-house buffers are best suited for spin column purification of aflibercept. Thus, it was decided to carry out a linearity study using different column loads to determine the optimal column load.

### Example 10. Column Load of Optimized Aflibercept Purification Method

The aim of the linearity study was to define the maximum and minimum column loading values that would result in comparable product quality to the routine small-scale purification. A column load range of 100.5 μg-804 μg was investigated for this purpose. The load volume was calculated based on a harvest titer of 1.34 mg/ml and the desired column load protein required. The step yield was calculated by dividing the pool total protein by the load total protein. The pool total protein was calculated using the elution pool volume, which was determined by weighing the elution tubes before and after elution, and the elution pool concentration, which was determined by UV-visible spectrometry. Table 35 shows the step yield obtained for eluate 1. Moreover, since the eluate 1 triplicates were combined and submitted for testing, the triplicate average was taken for the step yields.

TABLE 35
Eluate 1 triplicate average step yield
results from the linearity study.

| | | Pool total | | Step yield |
|---|---|---|---|---|
| Load total | Eluate 1 | protein | Step yield | eluate 1 |
| protein | concentration | eluate 1 | eluate 1 | (%) triplicate |
| (μg) | (mg/mL) | (mg) | (%) | averages |
| 804 | 1.35391 | 0.56 | 69.75 | 66.78 |
| | 1.41652 | 0.58 | 72.45 | |
| | 1.15012 | 0.47 | 58.14 | |
| 603 | 1.13826 | 0.48 | 79.38 | 78.09 |
| | 1.12586 | 0.48 | 79.95 | |
| | 1.07154 | 0.45 | 74.95 | |
| 402 | 0.80361 | 0.34 | 84.26 | 81.27 |
| | 0.79015 | 0.33 | 82.83 | |
| | 0.72593 | 0.31 | 76.73 | |
| 201 | 0.33705 | 0.14 | 71.47 | 73.9 |
| | 0.34648 | 0.15 | 73.33 | |
| | 0.36488 | 0.15 | 76.92 | |
| 100.5 | 0.19827 | 0.08 | 80.98 | 71.02 |
| | 0.15178 | 0.06 | 62.98 | |
| | 0.16679 | 0.07 | 69.09 | |

TABLE 36
Eluate 2 triplicate average step yield
results from the linearity study.

| | | Pool total | | Step yield |
|---|---|---|---|---|
| Load total | Eluate 2 | protein | Step yield | eluate 2 |
| protein | concentration | eluate 2 | eluate 2 | (%) triplicate |
| (μg) | (mg/mL) | (mg) | (%) | averages |
| 804 | 0.45433 | 0.19 | 24.03 | 21.99 |
| | 0.35774 | 0.15 | 18.81 | |
| | 0.43804 | 0.19 | 23.12 | |
| 603 | 0.2636 | 0.1 | 17.23 | 18.1 |
| | 0.25935 | 0.11 | 18.13 | |
| | 0.26926 | 0.11 | 18.94 | |
| 402 | 0.18689 | 0.08 | 19.87 | 19.54 |
| | 0.15724 | 0.07 | 16.68 | |
| | 0.20881 | 0.09 | 22.06 | |
| 201 | 0.13933 | 0.06 | 29.6 | 27.34 |
| | 0.13116 | 0.06 | 27.82 | |
| | 0.11538 | 0.05 | 24.59 | |
| 100.5 | 0.0641 | 0.03 | 26.34 | 29.22 |
| | 0.05772 | 0.02 | 23.94 | |
| | 0.0892 | 0.04 | 37.38 | |

TABLE 37
Triplicate average step yield from the linearity study.

| Load total | Total | Step | Triplicate |
|---|---|---|---|
| protein (μg) | pool (mg) | Yield (%) | Average (%) |
| 804 | 0.75 | 93.78 | 88.77 |
| | 0.73 | 91.26 | |
| | 0.65 | 81.26 | |
| 603 | 0.58 | 96.6 | 96.19 |
| | 0.59 | 98.08 | |
| | 0.57 | 93.89 | |
| 402 | 0.42 | 104.12 | 100.81 |
| | 0.4 | 99.51 | |
| | 0.4 | 98.79 | |
| 201 | 0.2 | 101.07 | 101.24 |
| | 0.2 | 101.15 | |
| | 0.2 | 101.5 | |
| 100.5 | 0.11 | 107.33 | 100.24 |
| | 0.09 | 86.92 | |
| | 0.11 | 106.47 | |

As shown in Table 35, the lowest step yield percentage obtained for eluate 1 was 66.78% when a total column load of 804 μg was applied and the highest step yield percentage obtained was 81.27% when a total column load of 402 μg was applied. One reason the eluate 1 step yield was not 100% can be attributed to the fact that less time was taken for incubation during the elution step, as the elution buffer could not effectively act in a short period of time to release the molecule bound to Protein A. The eluate 2 step yields as seen in Table 36 ranged from 18.10% to 29.22% when a total column load of 603 μg and 100.5 μg, respectively, was applied, which shows that most of the remaining bound molecules was successfully eluted upon addition of the elution buffer the second time. The average step yield ranged from 88.77% to 101.24% (Table 37). The lowest step yield average, 88.77%, was obtained when 804 μg of protein was loaded onto the spin column. One reason for the lower step yield could be the higher total protein concentration used, as it was possible that protein A did not have any available binding sites for the molecules to bind to, causing the molecules to flow through during the binding step.

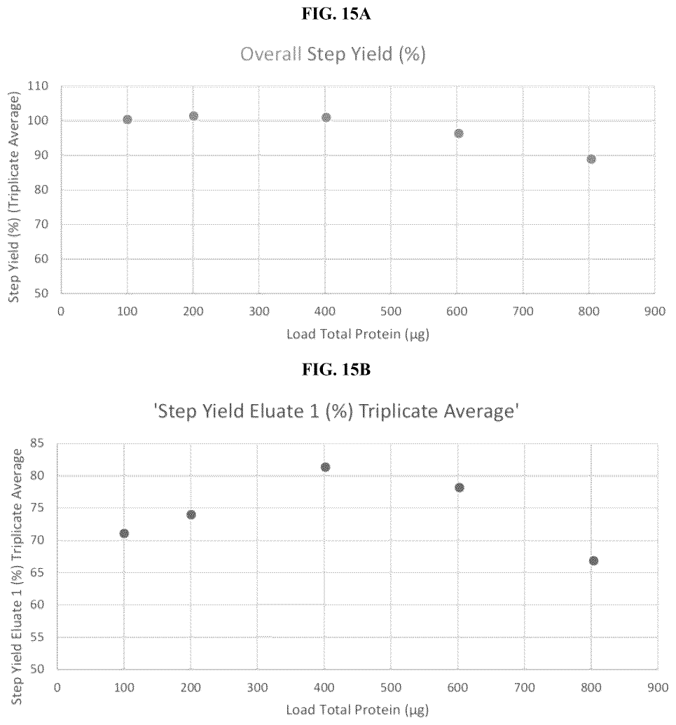

FIG. 15A shows a graphical representation of the average step yield percentage for each column loading value. FIG. 15B shows a graphical representation of the average step yield percentage of eluate 1 for each column loading value. As evidenced by FIG. 15A, a total column load as low as 100.5 μg yielded optimal step yield. Optimal step yield was also obtained for a total column load of 201 μg, 402 μg, and 603 μg. Since a satisfactory step yield was obtained for all tested column loading values, eluate 1 was analyzed for aggregation and fragmentation by SE-UPLC and charge by iCIEF, and compared to the eluate obtained from the established small-scale purification method.

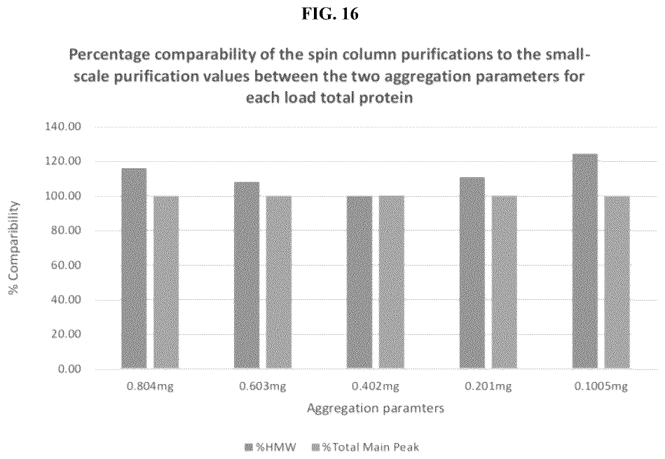

As shown in Table 38 and FIG. 16, the total main peak of the spin column purified sample were highly comparable to that of the small-scale purified sample. Variability in the percentage comparability was observed for the HMW species, ranging from −0.35% to +24.31%. The parentage comparability of the HMW species for the spin column purified sample was higher in comparison to the small-scale purified sample for each total protein load expect 402 μg. One reason for this variability could be sample manipulation due to freeze thaw cycles, as the samples were thawed to some extent for product quality testing.

TABLE 38
Comparison of CE-SDS and icIEF results between
samples purified using spin column method using
in-house buffers and small-scale method.

| | Comparability (%) | | | | |
|---|---|---|---|---|---|
| Load total | Aggregation | | | | |
| protein | | % Total | Charge | | |
| (μg) | % HMW | Main Peak | Region 1 | Region 2 | Region 3 |
| 804 | 115.97 | 99.56 | 111.3 | 101.78 | 89.46 |
| 603 | 108.04 | 99.71 | 111.99 | 105.92 | 85.14 |
| 402 | 99.65 | 100.01 | 113.7 | 104.73 | 84.86 |
| 201 | 110.8 | 99.81 | 114.38 | 104.14 | 84.86 |
| 100.5 | 124.31 | 99.58 | 117.47 | 104.14 | 82.7 |

The charge variant profile of the spin column purified sample was found to vary among the three regions, with the main species (region 2) of the spin column purified sample being the most comparable to the small-scale purified sample (percentage comparability ranged from +1.78% to +5.92% across the different column loading values). The percentage comparability of the acidic (region 1) and basic (region 3) variants were higher and lower, respectively, for the spin column purified sample than the small-scale purified sample (percentage comparability of region 2 ranged from +11.3% to +17.47% and region 3 ranged from −10.54% to −17.3%). The inverse relationship between region 1 and 3 is expected as any change in one region will be reflected in one or both regions due to the assay results from icIEF being based on percentage calculation.

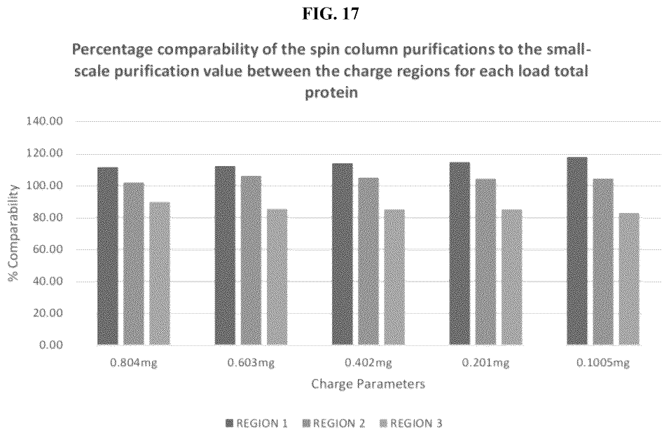

As evidenced by FIG. 17, one possible reason for variability in the charge variant profile may be due to the different protein A resin used for the purification, as the different protein A resins could have influenced the charge variants observed in eluate 1. The remaining charge variants could have eluted in eluate 2, but were not accounted for since only eluate 1 samples were sent for testing.

Overall, the developed spin column purification method demonstrated high comparability in terms of step yield, aggregation, and charge. Some differences in the spin column purified sample, particularly in the charge variant profile, could be attributed to the type of protein A resin employed in the spin column purification method compared to the small-scale purification method. The spin column purification method will reduce the time required for purifying samples from over multiple days to about 30 minutes. Moreover, the low sample requirement is beneficial for samples produced using small-scale bioreactors or at early stages of the cell culture when protein concentration is very low, allowing for multi time point sample studies for product quality analysis.

### Example 11. Time Course Sample Purification of Aflibercept Using Spin Columns

While a higher yield was obtained when a total column load of 400 μg was loaded onto the spin column (see Table 35), a column loading of 600 μg was selected for the time course study, as it provided higher protein concentration for eluate 1 and comparable product quality results to the 400 μg column loading condition.

Eluate 1 for each time point was submitted for aggregation and charge testing. For data analysis, the product quality results from the spin column purified samples were normalized against the results from the harvest material purified using the same spin column purification column. The percentage comparability of the product quality results of the time point samples to the harvest are shown in Table 39.

TABLE 39
Comparison of CE-SDS and icIEF results between time point samples
purified using the spin column purification method and harvest
material purified using the spin column purification method.

| | Comparability (%) | | | | |
|---|---|---|---|---|---|
| | Aggregation | | | | |
| Time | | % Total | Charge | | |
| Point | % HMW | Main Peak | Region 1 | Region 2 | Region 3 |
| A | 71.92 | 100.26 | 81.49 | 123.62 | 94.54 |
| B | 29.88 | 101.26 | 96.10 | 119.02 | 86.34 |
| C | 27.66 | 101.14 | 98.70 | 123.31 | 80.33 |
| D | 29.92 | 101.11 | 108.44 | 119.94 | 75.14 |
| E | 36.53 | 100.98 | 108.44 | 120.86 | 74.32 |
| F | 41.31 | 100.86 | 108.77 | 119.33 | 75.41 |
| G | 53.31 | 100.61 | 104.87 | 117.48 | 80.33 |
| H | 69.87 | 100.22 | 102.92 | 112.58 | 86.34 |
| I | 91.12 | 99.19 | 104.87 | 107.98 | 88.80 |
| J | 111.91 | 99.47 | 105.19 | 101.23 | 94.54 |

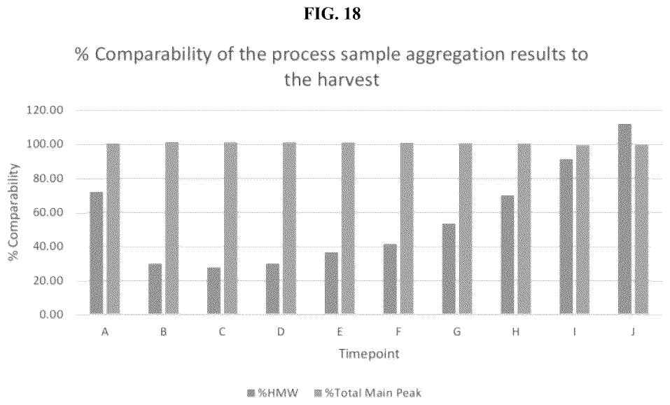

The % total main peak of the time point samples was highly comparable to the harvest material, with the percentage comparability ranging from −0.81% to +1.26% (Table 38 and FIG. 18). With the exception of the sample from time point A, the % HMW appeared to increase over time, with samples from time points I and J being the most comparable to the harvest material. This is expected due to several factors, including increased protein concentration during protein production, physicochemical stress, interaction with misfolded proteins, etc. (Kumari et al., 2023, Journal of Pharmaceutical and Biomedical Analysis, 18 (1): 126-132). However, the reason for the high % HMW for time point A is unclear.

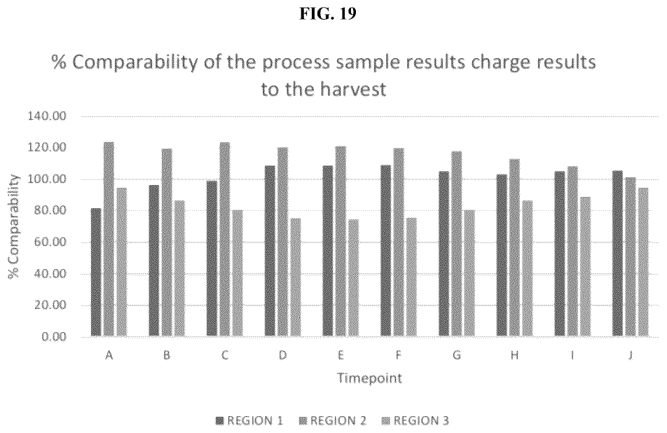

As shown in FIG. 19, the charge variant profile varied from time points A to J, suggesting that the charge variant profiles changes over the production process. Indeed, in a study conducted for CHO cell culture process optimization, variation in the charge variants as a result of the culture process was observed (Weng et al., 2020, Cytotechnology, 72 (2): 259-269). As shown in FIG. 19, the main species (region 2) appeared to decrease over time while the acidic and basic variants (region 1 and 3) appeared to increase over time, with the acidic and basic variants at time point J being highly comparable to the harvest material. The most noticeable change was observed starting from time point D, in which region 1 values appeared to stabilize over time while region 2 and 3 values changed in an inverse relationship.

Overall, these results demonstrate that multi-time point testing of samples can be used to understand changes in the product quality profile of aflibercept during the production process. This provides the unique potential to investigate the impact of product quality during the earlier stages of the production process, as product quality testing is normally carried out only after harvest. Moreover, this purification method can be used to rapidly purify and carry out product quality assessment if any deviations in product quality occur during the production process and a process related investigation is required.

### Example 12. Purification of Dupilumab and Aflibercept from Other Cell Types

Alternative purification processes of dupilumab and aflibercept may feature the use of other mammalian cell types, such as HEK 293 cells and baby hamster kidney (BHK) cells. Non-mammalian host cells lines may also be used, such as Sf9 insect cell lines. Different cell lines may used with minor modifications to the cell culture medium and purification process consistent with the description herein. Despite these slight modifications in the purification procedure, it is expected that the small-scale spin column purification method will yield antibodies with a comparable product quality profile to the large-scale purification method.

For example, a high-glucose growth medium, such as Dulbecco's Modified Eagle's Medium (DMEM) or Eagle's Minimum Essential Medium Eagle (MEM), supplemented with 5 to 10% fetal bovine serum (FBS), or serum-free/chemically defined media, such as EX-CELL® 293 serum-free medium or Gibco™ CD BHK-21 production medium, may be used to culture HEK 293 or BHK cells. TNM-FH-supplemented Grace's Insect Medium supplemented with glutamine and 10% FBS may be used to culture Sf9 insect cells. The binding/wash buffer may be modified with a different pH and/or ionic strength to ensure all the excess and unbound components are washed from the Protein A spin column. The binding/wash buffer may include buffering agents, such as sodium phosphate, sodium acetate, HEPES, or Tris, and additional salts, such as sodium chloride or calcium chloride. The pH of the binding/wash buffer may range from pH 6 to 8. Purification using the optimized developed spin column method using cell types, such as HEK 293 cells and BHK cells and insect cell types, with the modified cell culture media and buffers should result in protein gains listed in Table 40 below:

TABLE 40
Concentration of spin column eluates measured with UV-visible
spectroscopy and the calculated protein mass gained using
alternative cell types and cell culture media.

| | Concentration | | | Expected |
|---|---|---|---|---|
| | (μg/μL) | Conc. - | | Protein |
| | (UV-visible | blank conc. | Volume | gained |
| Sample | spectroscopy) | (μg/μL) | (μL) | (μg) |
| Blank elution 1 | 0.05 | 0 | 400 | 0 |
| Column 1 elution 1 | 0.9 | 0.8-0.9 | 400 | 340-350 |
| Column 2 elution 1 | 0.8 | 0.7 | 400 | 290 310 |
| Column 3 elution 1 | 0.8 | 0.7 | 400 | 280-300 |
| Blank elution 2 | 0.01 | 0 | 400 | 0 |
| Column 1 elution 2 | 0.4 | 0.4 | 400 | 140-160 |
| Column 2 elution 2 | 0.5 | 0.5 | 400 | 175-200 |
| Column 3 elution 2 | 0.5 | 0.5 | 400 | 190-210 |

Currently, small-scale laboratory studies on cell culture material require setting up multiple scaled down bioreactors to obtain samples at varying stages of the upstream production process. This is because a relatively large sample volume is needed for purification using the established large-scale method, and the number of sampling points from a small-scale bioreactor is limited. As a result, such studies presently require not only significant workload but also significant investment of time.

It was demonstrated that the newly developed small-scale purification method yields antibodies with a product quality profile comparable to the established large-scale method at the Protein A step that can be carried out in a reduced timeframe (about 30 minutes compared to multiple hours-usually over the course of 2 workdays) and with smaller volumes of buffers and the sample material itself, depending on sample concentration. The much higher efficiency of the small-scale method allows for cell culture samples to be purified daily throughout the manufacturing process, even in small-scale studies. Analyzing daily samples facilitates near-real-time or at-line monitoring of product quality attributes of the produced antibody, resulting in improved process understanding.

Daily cell culture samples significantly increase in recombinant protein concentration with each passing day, as the CHO cells continue to secrete the protein of interest. However, only harvest samples with a relatively high concentration (approximately 7 mg/mL, varying between batches) are often purified and analyzed. In the experiments disclosed in the Examples, daily samples with lower protein concentrations were purified using the developed spin column method. The conducted studies demonstrate that even a column load of 200 μg, three times less than the observed optimal column load of 600 μg, delivers an acceptable average step yield. As the purification method is done for testing purposes, maximizing the % recovery is not crucial. However, the % yield affects the final concentration of the purified mAb, which is eluted twice with 400 μL of the elution buffer. Additional studies were conducted to purify proteins from mini bioreactor harvest material using the spin column purification method, and the samples obtained from this purification method was found to give comparable product quality results to that of the small-scale purified samples, which demonstrates the suitability of the method to be used with mini bioreactor harvest material.

In the future, additional studies will be conducted on the column load range of 50-200 μg to investigate the protein yield obtained with the small-scale method when smaller sample amounts are added. Low-concentration samples collected during early stages of the manufacturing process (particularly day 1 and day 2) may require purification of volumes larger than the maximum capacity of the spin columns (600 μL) to reach the desirable column load. In that case, the antibody binding step may be carried out more than once to allow a larger sample volume to pass through the column. Additionally, if the purified early-day mAb sample concentration is lower than optimal for PQ testing due to a lowered column load and % yield, the volume of elution buffer used during the two elution steps may be decreased. Alternatively or additionally, centrifugal filters may be used to concentrate the purified samples.

The ability to analyze cell culture samples daily throughout the upstream process may also improve reactions to unplanned events which potentially influence the performance of the process. Investigating such events early in the batch duration by daily testing following purification with the developed small-scale method may help determine the potential impacts within a day of the event occurring. Using conventional methods, the upstream production process (which takes about 2 weeks, depending on the mAb) would need to be completed before harvest cell culture samples were purified and analyzed. Daily testing therefore could save both time and costs associated by introduction of the capability to monitor PQ changes in relation to process inputs.

The methods and systems of the present invention may also be used to build a data library of a monoclonal antibody's product quality attribute profile throughout the upstream production process. This may be achieved through continuous collection of data for successive large-scale manufacturing batches. The correlated product quality data can then be linked to process parameters such as pH, temperature, feeding strategy, or the concentration of cell culture components. This would allow for the effect of variations in the process parameters on the PQ profile to be established. Such understanding would support process-related investigation in the event of unplanned parameter changes (e.g. a drop in pH), and could potentially be used to adjust process parameters as needed to obtain a product with a desired PQ profile.

The methods and systems of the present invention may be applied to any antibody or Fc-containing protein, such as an Fc-receptor fusion protein, when using a Protein A resin. Other proteins of interest may be purified using a corresponding affinity resin, for example an affinity resin comprising a target ligand. Additional studies will include using the spin column purification method as a reference for developing purification methods for other molecules, and developing the same approach for other purification types (e.g., ion exchange chromatography).

Furthermore, the developed method is amenable to automation, and could be adapted to involve reduced operator workload and facilitate the purification of large numbers of samples. The methods and systems of the present invention may be applied as part of an end-to-end purification and product quality analysis platform. The procedure can be automated with a robotic liquid handling system, and the testing of multiple samples can be carried out using 96-well batch chromatography (Rathore and Bhambure; Lambiase et al., 2023, J. Chromatog. A, 463809).

Currently, out of the product quality testing described in this disclosure, only glycan profile analysis is automated using a commercially available pipetting robot. However, fragmentation testing could easily be transferred to that platform. Numerous automated high-throughput purification and multi-attribute method (MAM) platforms which incorporate the analysis of multiple cPQ attributes have recently been designed and documented in published literature (Sitasuwan et al., 2021, mAbs, 13 (1): 1978131; Yang et al., 2023, mAbs, 15 (1): 2197668; Lambiase et al., 2022, J. Chromatogr. A, 1670:462944; Liu et al., 2021, J. Pharm. Sci., 111 (2): 358-367). Additionally, automated systems for sampling directly from upstream bioreactors coupled to the MAM analytical platform for the purpose of online monitoring have also been described (Liu et al.; Dahotre et al., 2022, J. Chromatogr. A, 1672:463067).

This disclosure describes a high-throughput method for the purification of a monoclonal antibody from upstream cell culture material suitable for the purpose of monitoring the critical product quality attributes (including aggregation, fragmentation, charge variants and glycan profile) of a product. Samples purified using the developed method were found to have a product quality profile comparable to samples purified using the established large-scale downstream method.

What is claimed is:

1. A method for enriching a protein of interest from a cell culture sample, comprising:
(a) contacting a cell culture sample including a protein of interest to a centrifugal column including an affinity resin to produce an immobilized sample, wherein said affinity resin specifically binds to said protein of interest;
(b) subjecting said immobilized sample to at least one washing step; and
(c) subjecting said immobilized sample from (b) to at least one elution step to produce an enriched protein of interest.

2. The method of claim 1, wherein said protein of interest is selected from a group consisting of a therapeutic protein, a receptor, an antigen-binding protein, an antibody, a monoclonal antibody, a multispecific antibody, a bispecific antibody, an antibody-derived protein, a fusion protein, a receptor fusion protein, a trap protein, a fragment thereof, a variant thereof, and a combination thereof.

3. The method of claim 1, wherein said protein of interest is a monoclonal antibody.

4. The method of claim 1, wherein said protein of interest is dupilumab.

5. The method of claim 1, wherein said protein of interest is aflibercept.

6. The method of claim 1, wherein said cell culture sample is from a mammalian cell culture or an insect cell culture.

7. The method of claim 1, wherein said cell culture sample is from a CHO cell culture, a CHO-K1 cell culture, a BHK cell culture, a HEK 293 cell culture, a Sf9 insect cell culture, or a variation thereof.

8. The method of claim 1, wherein said cell culture sample is a clarified cell culture sample.

9. The method of claim 1, wherein said cell culture sample is taken from a cell culture at a day from day 1 to day 20, day 3 to day 15, day 3 to day 12, day 3 to day 10, day 5 to day 10, day 5 to day 12, or day 5 to day 13.

10. The method of claim 1, wherein said cell culture sample is taken from a cell culture at a day selected from a group consisting of day 1, 2, 3, 4, 5, 6, 7, 8, 9, 10, 11, 12, 13, 14, 15, 16, 17, 18, 19, or 20.

11. The method of claim 1, further comprising repeating the method at least once.

12. The method of claim 11, wherein the method is repeated using at least a first cell culture sample and a second cell culture sample taken from the same cell culture.

13. The method of claim 12, wherein said first cell culture sample is taken at a first day and said second cell culture sample is taken at a second day.

14. The method of claim 12, wherein said first cell culture sample and said second cell culture sample are taken with a time between samples of about 3 hours, about 6 hours, about 12 hours, about 18 hours, about 24 hours, about 1 day, about 2 days, about 3 days, about 4 days, about 5 days, about 6 days, about 7 days, about 8 days, about 9 days, or about 10 days.

15. The method of claim 1, further comprising performing the method with at least two cell culture samples in parallel.

16. The method of claim 15, wherein said at least two cell culture samples are from two different cell cultures.

17. The method of claim 1, wherein said contacting step comprises combining said cell culture sample and a binding buffer.

18. The method of claim 17, wherein said binding buffer comprises Tris-buffered saline, sodium phosphate, HEPES, or Tris.

19. The method of claim 18, wherein said binding buffer further comprises sodium chloride or calcium chloride.

20. The method of claim 17, wherein said binding buffer comprises sodium phosphate.

21. The method of claim 20, wherein said binding buffer further comprises sodium chloride.

22. The method of claim 17, wherein a pH of said binding buffer is from about 6 to about 8.

23. The method of claim 1, wherein said contacting step comprises adding to said column a combined volume of a binding buffer and said cell culture sample of from 250 to 1000 μL, from 300 to 900 μL, from 400 to 800 μL, from 500 to 700 μL from 550 to 650 μL, from 590 to 610 μL, from 599 to 601 μL, or about 600 μL.

24. The method of claim 1, wherein said contacting step comprises adding to said column a volume of said cell culture sample of from 50 to 100 μL, from 60 to 90 μL, from 70 to 80 μL, about 70 μL, about 71 μL, about 72 μL, about 73 μL, about 74 μL, about 75 μL, about 76 μL, about 77 μL, about 78 μL, about 79 μL, or about 80 μL.

25. The method of claim 1, wherein said contacting step comprises adding to said column an amount of protein of from 100.5 μg to 804 μg, 250 μg to 1 g, from 350 μg to 900 μg, from 450 μg to 804 μg, from 500 μg to 700 μg, from 550 μg to 650 μg, from 575 μg to 625 μg, from 590 μg to 610 μg, about 595 μg, about 596 μg, about 597 μg, about 598 μg, about 599 μg, about 600 μg, about 601 μg, about 602 μg, about 603 μg, about 604 μg, or about 605 μg.

26. The method of claim 1, wherein said affinity resin is Protein A resin, Protein G resin, or a combination thereof.

27. The method of claim 1, wherein said at least one washing step comprises adding a washing buffer to said column and centrifuging said column to produce a washed flowthrough.

28. The method of claim 27, wherein said washing buffer comprises Tris-buffered saline, sodium phosphate, sodium acetate, HEPES, or Tris.

29. The method of claim 28, wherein said washing buffer further comprises sodium chloride or calcium chloride.

30. The method of claim 27, wherein said washing buffer comprises sodium phosphate or sodium acetate.

31. The method of claim 30, wherein said washing buffer further comprises sodium chloride.

32. The method of claim 27, wherein a pH of said washing buffer is from about 6 to about 8.

33. The method of claim 1, wherein a number of washing steps is one, two, or three.

34. The method of claim 1, wherein a number of washing steps is two.

35. The method of claim 34, wherein the washing buffer of said first washing step comprises sodium phosphate and sodium chloride, and the washing buffer of said second washing step comprises sodium acetate.

36. The method of claim 27, wherein a volume of said washing buffer is about 600 μL.

37. The method of claim 27, wherein said centrifuging is performed at about 100 relative centrifugal force (RCF).

38. The method of claim 27, wherein said centrifuging is performed for about 1 minute.

39. The method of claim 1, wherein said at least one elution step comprises adding an elution buffer to said column and centrifuging said column to produce an eluate.

40. The method of claim 39, wherein said elution buffer comprises acetic acid or glycine.

41. The method of claim 39, wherein a concentration of said acetic acid is about 0.24% or about 40 mM.

42. The method of claim 39, wherein a concentration of said acetic acid is about 0.12% or about 20 mM

43. The method of claim 39, wherein a concentration of said glycine is about 0.1 M.

44. The method of claim 39, wherein a volume of said elution buffer is about 400 μL.

45. The method of claim 39, wherein a pH of said elution buffer is from 1 to 4, from 2 to 4, from 2.5 to 3.5, from 2.8 to 3.2, about 1, about 1.5, about 2, about 2.5, about 3, or about 3.5.

46. The method of claim 1, wherein a number of elution steps is one, two, or three.

47. The method of claim 39, wherein said centrifuging is performed at about 100 RCF.

48. The method of claim 39, wherein said centrifuging is performed for about 1 minute.

49. The method of claim 1, wherein said at least one elution step comprises adding a neutralizing buffer to said column.

50. The method of claim 49, wherein said neutralizing buffer comprises Tris base.

51. The method of claim 50, wherein a concentration of said Tris base is from 1 M to 2 M, about 1 M, about 1.5 M, or about 2 M.

52. The method of claim 49, wherein a volume of said neutralizing buffer is from 5 μL to 50 μL, about 5 μL, about 10 μL, about 20 μL, about 30 μL, about 40 μL, or about 50 μL.

53. The method of claim 1, wherein a yield of said enriched protein of interest is above 50%, above 60%, above 70%, above 80%, above 90%, above 95%, above 99%, about 60%, about 70%, about 75%, about 80%, about 85%, about 90%, about 91%, about 92%, about 93%, about 94%, about 95%, about 96%, about 97%, about 98%, about 99%, or about 100%.

54. The method of claim 1, wherein an amount of protein in said enriched protein of interest is above 10 μg, above 20 μg, above 50 μg, above 100 μg, above 200 μg, above 300 μg, above 400 μg, above 500 μg, above 600 μg, above 700 μg, above 800 μg, above 900 μg, above 1000 μg, above 1100 μg, above 1200 μg, about 10 μg, about 20 μg, about 50 μg, about 100 μg, about 200 μg, about 300 μg, about 400 μg, about 500 μg, about 600 μg, about 700 μg, about 800 μg, about 900 μg, about 1000 μg, about 1100 μg, or about 1200 μg.

55. The method of claim 1, wherein a concentration of said enriched protein of interest is above 0.01 μg/μL, above 0.05 μg/μL, above 0.1 μg/μL, above 0.2 μg/μL, above 0.5 μg/μL, above 1 μg/μL, above 2 μg/μL, about 0.05 μg/μL, about 0.1 μg/μL, about 0.2 μg/μL, about 0.5 μg/μL, about 1 μg/μL, about 1.5 μg/μL, about 2 μg/μL, or about 2.5 μg/μL.

56. The method of claim 1, wherein a duration of said method is less than 24 hours, less than 12 hours, less than 6 hours, less than 3 hours, less than 2 hours, less than 1 hour, less than 30 minutes, about 3 hours, about 2 hours, about 1.5 hours, about 1 hour, about 50 minutes, about 45 minutes, about 40 minutes, about 30 minutes, or about 20 minutes.

57. The method of claim 1, further comprising characterizing at least one product quality attribute of said enriched protein of interest.

58. The method of claim 1, further comprising subjecting said enriched protein of interest to chromatography, mass spectrometry, spectroscopy, capillary electrophoresis, gel electrophoresis, and/or a ligand binding assay.

59. The method of claim 1, further comprising characterizing at least one size variant of said enriched protein of interest.

60. The method of claim 1, further comprising characterizing at least one high molecular weight species of said enriched protein of interest.

61. The method of claim 60, wherein said characterizing comprises subjecting said enriched protein of interest to size exclusion chromatography (SEC) analysis.

62. The method of claim 1, further comprising characterizing at least one fragment of said enriched protein of interest.

63. The method of claim 62, wherein said characterizing comprises subjecting said enriched protein of interest to capillary electrophoresis with sodium dodecyl sulfate (CE-SDS) analysis.

64. The method of claim 1, further comprising characterizing at least one charge variant of said enriched protein of interest.

65. The method of claim 64, wherein said characterizing comprises subjecting said enriched protein of interest to imaged capillary isoelectric focusing electrophoresis (iCIEF).

66. The method of claim 1, further comprising characterizing at least one glycan of said enriched protein of interest.

67. The method of claim 66, wherein said characterizing comprises subjecting said enriched protein of interest to hydrophilic interaction chromatography (HILIC) analysis.

68. The method of claim 57, further comprising using said at least one product quality attribute to determine whether said cell culture should be continued or discontinued.

69. The method of claim 57, further comprising using said at least one product quality attribute to determine whether said cell culture should be modified.
